SEQUENCE LISTING (3 sequences)

SEQ ID NO: 1 - DNA, 21 bp, Artificial Sequence
  Probe
cccaggccatctcggaaacgc

SEQ ID NO: 2 - DNA, 23 bp, Artificial Sequence
  Primer
ctgcaccacaacgtggttttcgt

SEQ ID NO: 3 - DNA, 18 bp, Artificial Sequence
  Primer
acggccaccgtcacattc

(19) United States
(12) Patent Application Publication
(10) Pub. No.: US 2011/0275644 A1
(43) Pub. Date: Nov. 10, 2011

(54) BIOLOGICAL MARKERS PREDICTIVE OF ANTI-CANCER RESPONSE TO INSULIN-LIKE GROWTH FACTOR-1 RECEPTOR KINASE INHIBITORS

(76) Inventors: Elizabeth A. Buck, Huntington, NY (US); David M. Epstein, Huntington, NY (US); Mark R. Miglarese, Superior, CO (US)

(21) Appl. No.: 13/039,562

(22) Filed: Mar. 3, 2011

Related U.S. Application Data
(60) Provisional application No. 61/310,031, filed on Mar. 3, 2010.

(51) Int. Cl. A61K 31/4985; A61P 35/00; C12Q 1/48

(52) U.S. Cl. 514/249; 435/15

(57) ABSTRACT

The present invention provides diagnostic methods for predicting the effectiveness of treatment of a cancer patient with an IGF-1R kinase inhibitor that inhibits both IGF-1R and IR kinases. Methods are provided for identifying patients with cancer who are likely to benefit from treatment with an IGF-1R kinase inhibitor that inhibits both IGF-1R and IR kinases. Methods are also provided for identifying patients with cancer who are likely to benefit from treatment with an IGF-1R kinase inhibitor that inhibits both IGF-1R and IR kinases, but who would likely not respond to therapy with an anti-IGF-1R antibody. Methods are also provided for identifying patients with cancer who are more likely to benefit from treatment with anti-IGF-1R antibody. Improved methods for treating cancer patients with IGF-1R kinase inhibitors that incorporate these methods are also provided.

### CROSS-REFERENCE TO RELATED APPLICATIONS

This application claims the benefit of U.S. Provisional Application No. 61/310,031, filed Mar. 3, 2010, which is herein incorporated by reference in its entirety.

### BACKGROUND OF THE INVENTION

Cancer is a generic name for a wide range of cellular malignancies characterized by unregulated growth, lack of differentiation, and the ability to invade local tissues and metastasize. These neoplastic malignancies affect, with various degrees of prevalence, every tissue and organ in the body. The present invention is directed to methods for diagnosing and treating cancer patients. In particular, the present invention is directed to methods for determining which patients will most benefit from treatment with an insulin-like growth factor-1 receptor (IGF-1R) kinase inhibitor.

IGF-1R belongs to the insulin receptor family that includes the Insulin Receptor (IR), IGF-1R (homodimer), IGF-1R/IR (hybrid receptor), and IGF-2R (mannose 6-phosphate receptor). IGF-1R/IR hybrids act as homodimers, preferentially binding and signaling with IGFs. IR exists in two isoforms: IR-B (traditional insulin receptor) and IR-A (a fetal form which is re-expressed in selected tumors and preferentially binds IGF-II). IGF-2R is a non-signaling receptor that acts as a “sink” for IGF-II (Pollak M. N., et al. Nat Rev Cancer 2004 4:505-18). Six well-characterized insulin-like growth factor binding proteins (IGFBP-1 through -6) associate with IGF ligands to stabilize the IGFs and modulate their ability to bind the IGF-IR.

IGF-1R is a transmembrane RTK that binds primarily to IGF-1 but also to 1GF-II and insulin with lower affinity. Binding of IGF-1 to its receptor results in activation of it's tyrosine kinase activity, intermolecular receptor autophosphorylation, and phosphorylation of cellular substrates, including IRS1 and Shc, leading to activation of the PI3K/Akt and mitogen-activated protein kinase (MAPK) pathways (Adams T. E., et al. Cell Mol Life Sci 2000 57:1050-93; Pollak M. N., et al. Nat Rev Cancer 2004 4:505-18; Baserga R., Exp Cell Res 1999 253:1-6). The ligand-activated IGF-1R induces mitogenic activity in normal cells and plays an important role in abnormal growth. A major physiological role of the IGF-1 system is the promotion of normal growth and regeneration. Overexpressed IGF-1R (type 1 insulin-like growth factor receptor) can initiate mitogenesis and promote ligand-dependent neoplastic transformation. Furthermore, IGF-1R plays an important role in the establishment and maintenance of the malignant phenotype. Unlike the epidermal growth factor (EGF) receptor, no mutant oncogenic forms of the IGF-1R have been identified. However, several oncogenes have been demonstrated to affect IGF-1 and IGF-1R expression. A correlation between a reduction of IGF-1R expression and resistance to transformation has been seen. Exposure of cells to mRNA antisense to IGF-1R RNA prevents soft agar growth of several human tumor cell lines. IGF-1R abrogates progression into apoptosis, both in vivo and in vitro. It has also been shown that a decrease in the level of IGF-1R below wild-type levels causes apoptosis of tumor cells in vivo. The ability of IGF-1R disruption to cause apoptosis appears to be diminished in normal, non-tumorigenic cells.

The IGF-1 pathway has an important role in human tumor development. IGF-1R overexpression is frequently found in various tumors (breast, colon, lung, sarcoma) and is often associated with an aggressive phenotype. High circulating IGF1 concentrations are strongly correlated with prostate, lung and breast cancer risk. Furthermore, IGF-1R is required for establishment and maintenance of the transformed phenotype in vitro and in vivo (Baserga R. Exp. Cell. Res., 1999, 253, 1-6). The kinase activity of IGF-1R is essential for the transforming activity of several oncogenes: EGFR, PDGFR, SV40 T antigen, activated Ras, Raf, and v-Src. The expression of IGF-1R in normal fibroblasts induces neoplastic phenotypes, which can then form tumors in vivo. IGF-1R expression plays an important role in anchorage-independent growth. IGF-1R has also been shown to protect cells from chemotherapy-, radiation-, and cytokine-induced apoptosis. Conversely, inhibition of endogenous IGF-1R by dominant negative IGF-1R, triple helix formation or antisense expression vector has been shown to repress transforming activity in vitro and tumor growth in animal models. The IGF-1R signaling pathway also appears to be a robust target in colorectal cancer (CRC), based upon data demonstrating overexpression of the receptor and ligands in CRC, association with a more malignant phenotype, chemotherapy resistance, and correlation with a poor prognosis (Saltz, L. B., et al. J Clin Oncol 2007; 25(30): 4793-4799; Tripkovic I., et al. Med Res. 2007 July; 38(5):519-25, Epub 2007 Apr. 26; Miyamoto S., et al. Clin Cancer Res. 2005 May 1;11(9):3494-502; Nakamura M., et al. Clin Cancer Res. 2004 Dec. 15; 10(24):8434-41; Grothey A, et al. J Cancer Res Clin Oncol. 1999; 125(3-4):166-73).

It has been recognized that inhibitors of protein-tyrosine kinases are useful as selective inhibitors of the growth of mammalian cancer cells. For example, Gleevec™ (also known as imatinib mesylate), a 2-phenylpyrimidine tyrosine kinase inhibitor that inhibits the kinase activity of the BCR-ABL fusion gene product, has been approved by the U.S. Food and Drug Administration for the treatment of CML. The 4-anilinoquinazoline compound Tarceva™ (erlotinib HCl) has also been approved by the FDA, and selectively inhibits EGF receptor kinase with high potency. The development for use as anti-tumor agents of compounds that directly inhibit the kinase activity of IGF-1R, as well as antibodies that reduce IGF-1R kinase activity by blocking IGF-1R activation or antisense oligonucleotides that block IGF-1R expression, are areas of intense research effort (e.g. see Larsson, O. et al (2005) Brit. J. Cancer 92:2097-2101; Ibrahim, Y. H. and Yee, D. (2005) Clin. Cancer Res. 11:944s-950s; Mitsiades, C. S. et al. (2004) Cancer Cell 5:221-230; Camirand, A. et al. (2005) Breast Cancer Research 7:R570-R579 (DOI 10.1186/bcr1028); Camirand, A. and Pollak, M. (2004) Brit. J. Cancer 90:1825-1829; Garcia-Echeverria, C. et al. (2004) Cancer Cell 5:231-239; Sachdev D, and Yee D., Mol Cancer Ther. 2007 Jan;6(1):1-12; Hofmann F., and Garcia-Echeverria C., Drug Discov Today 2005 10:1041-7). Agents inhibiting the IGF-1R pathway have demonstrated anti-tumor efficacy in multiple human cancer models both in vitro and in vivo, particularly in pediatric models of Ewing's sarcoma and rhabdomyosarcoma (Manara M C, et al. Int J Oncol 2005 27:1605-16). Despite early hints of efficacy in patients with sarcoma, results to date of IGF-1R inhibitors in early clinical trials have not been impressive, indicating that patient selection strategies and rational combinations may be needed to move forward with this approach (Tolcher A. W., et al. Journal of Clinical Oncology, 2007 ASCO Annual Meeting Proceedings Part I. Vol 25, No. 18S (June 20 Supplement), 2007: 3002). Data acquired this far, has not indicated that activation, overexpression, or amplification of members of the IGF-1R pathway will predict responsiveness.

There is a need for both more efficacious treatment for neoplasia and other proliferative disorders, and for more effective means for determining which tumors will respond to which treatment. Several groups have investigated or disclosed potential biomarkers to predict a patient's response to protein-tyrosine kinase inhibitors (see for example, PCT publications: WO 2004/063709, WO 2005/017493, WO 2004/111273, WO 2008/108986, WO 2007/001868 and WO 2004/071572; and US published patent applications: US 2005/0019785, US 2007/0065858, US 2009/0092596, US 2009/0093488, US 2006/0140960 and US 2004/0132097). Several biomarkers have been proposed for predicting the response to EGFR kinase inhibitors, including mutant KRAS as a predictor of non-responsiveness in colorectal cancer (e.g. see Brugger, W. et al. (2009) J Clin Oncol 27:15s, (suppl; abstr 8020); Siena, S et al (2009) JNCI 101(19):1308-1324; Riely and Ladanyi (2008) J Mol Diagnostics 10(6):493; Jimeno, A. et al. (2009) Cancer J. 15(2):110-13). In addition, several biomarkers, including mutant KRAS, have been disclosed that have potential in predicting a patient's response to IGF-1R kinase inhibitors (e.g. see Rodon, J. et al (2008) Mol Cancer Ther. 7:2575-2588; T. Pitts et al. (2009) EORTC Conference, Boston, Mass., abstract #2141; Huang, F. et al. (2009) Cancer Res. 69(1):161-170; Rodon, J. et al., (2008) Mol. Cancer Ther. 7:2575-2588). However, in most instances no FDA-approved diagnostic tests have yet emerged that can effectively guide practicing physicians in the treatment of their patients with such inhibitors, or can indicate to the physician which tumors will respond most favorable to a combination of such an inhibitor with a standard chenmotherapy agent.

Thus, there remains a critical need for improved methods for determining the best mode of treatment for any given cancer patient. The present invention provides methods for determining which tumors will respond most effectively to treatment with IGF-1R kinase inhibitors that inhibit both IGF-1R and IR kinases, based on whether the tumor cells express certain levels of mRNA transcripts that are predictive of sensitivity to such IGF-1R kinase inhibitors, and for the incorporation of such biomarker determinations into more effective treatment regimens for cancer patients, whether such inhibitors are used as single agents or combined with other anti-cancer agents.

### SUMMARY OF THE INVENTION

The present invention provides diagnostic methods for predicting the effectiveness of treatment of a cancer patient with an IGF-1R kinase inhibitor that inhibits both IGF-1R and IR kinases. These methods are based on the surprising discovery that the sensitivity of tumor cell growth to inhibition by such IGF-1R kinase inhibitors is predicted by whether such tumor cells have a sufficiently high value of a gene expression level index comprising the sum of the expression levels of the five gene transcripts IGF-1R, IR, IR-A, IGF-1 and IGF-2. Whether the tumor cells have a sufficiently high value of the expression level index that is predictive of sensitivity is determined by assessing whether the index value is equal to or greater than a value of the expression level index determined to be a minimum value required to predict inhibitor sensitivity. The latter minimum value was determined by a study that established the relationship between tumor cell sensitivity to inhibitor and the expression level index, and provides reference tumor cell lines that can be used for comparison purposes to indicate the magnitude of this minimum value, e.g. RDES or SK—N-AS tumor cells.

Accordingly, the present invention provides a method of identifying patients with cancer who are likely to benefit from treatment with an IGF-1R kinase inhibitor that inhibits both IGF-1R and IR kinases, comprising: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than the value of the expression level index for RDES or SK—N-AS tumor cells determined by identical methods, the patient is likely to benefit from treatment with an IGF-1R kinase inhibitor that inhibits both IGF-1R and IR kinases.

Improved methods for treating cancer patients with IGF-1R kinase inhibitors that inhibit both IGF-1R and IR kinases that incorporate the above methodology are also provided. Thus, the present invention further provides a method for treating tumors or tumor metastases in a patient, comprising the steps of diagnosing a patient's likely responsiveness to an IGF-1R kinase inhibitor that inhibits both IGF-1R and IR kinases by assessing whether the tumor cells have a sufficiently high value of a gene expression level index comprising the sum of the expression levels of the five gene transcripts IGF-1R, IR, IR-A, IGF-1 and IGF-2, and administering to said patient a therapeutically effective amount of an IGF-1R kinase inhibitor that inhibits both IGF-1R and IR kinases (e.g. OSI-906) where responsiveness to the inhibitor is predicted.

The present invention also provides diagnostic methods for identifying patients with cancer who are likely to benefit from treatment with an IGF-1R kinase inhibitor that inhibits both IGF-1R and IR kinases, but would likely not respond to therapy with an anti-IGF-1R antibody, by combining the above described methodology with a determination of whether the tumor cells have a sufficiently high value of a gene expression level index comprising the sum of the expression levels of the gene transcripts IR and IR-A that is predictive of resistance to growth inhibition by an anti-IGF-1R antibody. Improved methods for treating cancer patients with IGF-1R kinase inhibitors that inhibit both IGF-1R and IR kinases that incorporate this methodology are also provided.

The present invention thus provides a method of identifying patients with cancer who are likely to benefit from treatment with an IGF-1R kinase inhibitor that inhibits both IGF-1R and IR kinases, but who would likely not respond to therapy with an anti-IGF-1R antibody, comprising: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than the value of the expression level index for RDES tumor cells as determined by identical methods, the patient is likely to benefit from treatment with an IGF-1R kinase inhibitor that inhibits both IGF-1R and IR kinases; and determining that if the value of the sum of expression levels for IR and IR-A for the tumor cells of the sample is equal to or greater than the sum of expression levels for IR and IR-A for GEO or A673 tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody.

The present invention also provides diagnostic methods for identifying patients with cancer who are not likely to benefit from treatment with anti-IGF-1R antibody, comprising determining whether the tumor cells of the patient express insulin receptor or phospho-IR, wherein if insulin receptor or phospho-IR is expressed, the tumor cells will be resistant to inhibition by the antibody. Improved methods for treating cancer patients with IGF-1R kinase inhibitors that incorporate these methods are also provided.

### BRIEF DESCRIPTION OF THE FIGURES

FIG. 1. Elevated expression of IGF receptor/ligand pairs is observed among tumor cell lines sensitive to OSI-906. A. Sensitivity to OSI-906 for a panel of 32 tumor cell lines derived from 10 tumor types, expressed as EC₅₀ values. Cell lines were categorized as either sensitive (EC₅₀<1 μM) or insensitive (EC₅₀>10 μM) to OSI-906. Mutational status for KRAS, BRAF, and PIK3CA is indicated, as reported by Sanger Wellcome database. Those mutation statuses that are not reported are shaded grey. B. Expression of IGFJ, IGF2, IGF1R, IR, and IRA mRNA by qPCR for the panel of 32 tumor cell lines. Gene expression was normalized to the fourth quartile expression for a given gene within the 32 cell line panel.

FIG. 2. Effect of varying concentrations of OSI-906 on cell growth for a representative panel of 5 sensitive tumor cell lines.

FIG. 3. The IGF-1R neutralizing antibody MAB391 confers a compensatory increase in IR phosphorylation, and co-targeting IGF-1R and IR achieves enhanced inhibition of the IRS1-AKT pathway for select tumor cells. A. Phosphorylation of IR and IGF-1R for a representative group of 8 human tumor cell lines (top panel, 1^{st} page). Effect of OSI-906 (3 μM) or MAB391 (3 μ/ml phosphorylation of IR and IGF-1R for a panel of 9 tumor cell lines categorized as sensitive to OSI-906 (2^{nd} page). Data are captured 16 hours after dosing and expressed as % of basal phosphorylation signal. A set of representative array images are shown for A673 Ewings sarcoma tumor cells (lower panel, 1^{st} page). B. Effect of 16 hour treatment with OSI-906 (3 μM) or MAB391 (3 μg/ml) on the phosphorylation of IR or IGF-1R, total IGF-1R expression, and phospho-AKT^{S437} or phospho-ERK for a panel of 4 tumor cell lines (1^{st} page: H322 and SK—N-AS; 2^{nd} page: H295R and A673). C. Effect of OSI-906 (3 μM) or MAB391 (3 μg/ml) on phospho-IRS-1^{Y612} for H295R, A673, and H322 cells. Also shown is phospho-AKT^{S473}, phospho-PRAS40, and total IGF-1R and IR levels under basal conditions or upon treatment with OSI-906 or MAB391 for H295R cells. Results shown are typical of 3 or more independent experiments.

FIG. 4. Xenograft tumors co-expressing pIGF-1R and pIR are sensitive to OSI-906 but not MAB391, while tumors expressing IGF-1R and not IR are sensitive to both OSI-906 and MAB391. A. Expression of IGF1, IGF2, IGF-1R, and IRA as determined by quantitative PCR and expression of phospho-IR and phospho-IGF-1R as determined by capture array (top panel, 1s^{t} page). Mice bearing SK—N-AS or GEO tumors were dosed with either OSI-906 (50 mg/kg qd) or MAB391 (1 mg/mouse q3d), as indicated (lower panel, 1^{st} page), and TG1 was determined over a 14 day period (2nd page). Effect of single dose OSI-906 or MAB391 on the phosphorylation of AKT for GEO and SK—N-AS tumors (3^{th} page). B. Effect of OSI-906 or MAB391 on the phosphorylation states for IR and IGF-1R in vivo for GEO tumors over the dosing period (i.e. 24 hours for OSI-906, or 72 hours for MAB391) (upper panel). Representative images are shown. Effects of MAB391 or OSI-906 on tumoral phospho-AKT^{S473} over the dosing period (lower panel).

FIG. 5. Insulin activation of tumor cell IR-AKT signaling is inhibited by OSI-906 but not MAB391. A. Effects on phosphorylation of IGF-1R and IR for HT-29 tumor cells treated with either OSI-906 (3 μM) or MAB391 (3 μg/ml) for 16 hours followed by stimulation with 50 μIU/ml for 5 minutes prior to cell lysis. B. Effect of OSI-906, MAB391, or IGFBP3 on phospho-AKT^{S473} for HT-29 cells under basal conditions or following stimulation with 5 or 50 μIU/ml insulin.

FIG. 6. MAB391 inhibits IGF-1, but not IGF-2 or insulin mediated stimulation of pIR. A. Effect of OSI-906 (3 μM) or MAB391 (3 μg/ml) on phospho-IR and phospho-IGF-1R for control cells or cells treated with insulin (50 μIU/ml), IGF-1 (40 ng/ml), or IGF-2 (40 ng/ml) for 5 minutes prior to lysis. Cartoon illustration of ligand-receptor binding pairs (right panel). B. Effect of OSI-906 or MAB391 on phospho-Akt^{S473} in the presence of IGF-1 or IGF-2. C. Effect of OSI-906 (3 μM), MAB391 (3 μg/ml), or an IGF-2 neutralizing antibody (10 μg/ml) on phosphorylation of IGF-1R or IR (left panel) or pPRAS40 (right panel) for MDAH-2774 cells following 16 hour treatment. Results shown are typical of 3 or more independent experiments. D. Cartoon illustrating the compensatory signaling through IR that can occur upon specific inhibition of IGF-1R.

FIG. 7: OSI-906 exhibits enhanced inhibition of AKT phosphorylation, compared to MAB391, in tumors that co-express phospho-IGF-1R and phospho-IR. A. Effect of a single dose of OSI-906 (50 mg/kg) or MAB391 (1 mg/mouse) on tumor AKT phosphorylation following 4 hours of treatment for SK—N-AS (A) and GEO (B) tumors. pAKT was determined by immunoblotting.

### DETAILED DESCRIPTION OF THE INVENTION

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

“Cell growth”, as used herein, for example in the context of “tumor cell growth”, unless otherwise indicated, is used as commonly used in oncology, where the term is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

“Tumor growth” or “tumor metastases growth”, as used herein, unless otherwise indicated, is used as commonly used in oncology, where the term is principally associated with an increased mass or volume of the tumor or tumor metastases, primarily as a result of tumor cell growth.

“Abnormal cell growth”, as used herein, unless otherwise indicated, refers to cell growth that is independent of normal regulatory mechanisms (e.g., loss of contact inhibition). This includes, for example, the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (3) any tumors that proliferate by receptor tyrosine kinases; (4 any tumors that proliferate by aberrant serine/threonine kinase activation; and (5) benign and malignant cells of other proliferative diseases in which aberrant serine/threonine kinase activation occurs.

The term “treating” as used herein, unless otherwise indicated, means to give medical aid to counteract a disease or condition. The phrase “a method of treating” or its equivalent, when applied to cancer refers to a procedure or course of action that is designed to reduce or eliminate the number of cancer cells in a patient, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed an overall beneficial course of action.

The term “therapeutically effective agent” means a composition that will elicit the biological or medical response of a tissue, system, animal or human that is being sought by the researcher, veterinarian, medical doctor or other clinician.

The term “therapeutically effective amount” or “effective amount” means the amount of the subject compound or combination that will elicit the biological or medical response of a tissue, system, animal or human that is being sought by the researcher, veterinarian, medical doctor or other clinician.

The terms “responsive”or “responsiveness” when used herein in referring to a patient's reaction to administration of an IGF-1R kinase inhibitor that inhibits both IGF-1R and IR kinases, refer to a response that is positive or effective, from which the patient is likely to benefit.

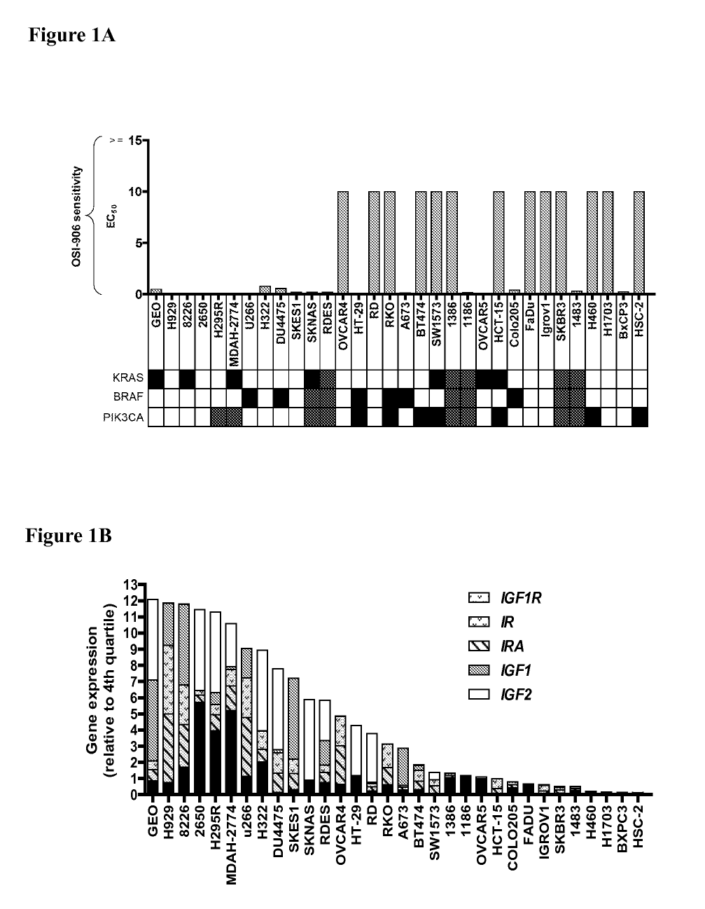

The data presented in the Experimental Details section herein below demonstrate that tumor cells have varying sensitivities to growth inhibition by an IGF-1R kinase inhibitor that inhibits both IGF-1R and IR kinases (e.g. OSI-906), with some tumor cells being relatively resistant to inhibition. It is demonstrated that the degree of sensitivity of tumor cells to such an IGF-1R kinase inhibitor can be assessed by determining whether tumor cells have a sufficiently high value of a gene expression level index comprising the sum of the expression level values of the five gene transcripts IGF-1R, IR, IR-A, IGF-1 and IGF-2. Whether the tumor cells have a sufficiently high value of the expression level index that is predictive of sensitivity is determined by assessing whether the index value is equal to or greater than a value of the expression level index determined to be a minimum value required to predict inhibitor sensitivity. This minimum value is the expression level index value associated with tumor cells such as RDES or SK—N-AS tumor cells. All tumor cells with an expression level index at or above this value are sensitive to an IGF-1R kinase inhibitor that inhibits both IGF-1R and IR kinases (e.g. see FIGS. 1A and 1B, which demonstrates this with a variety of tumor cell types with the IGF-1R kinase inhibitor OSI-906).

The data presented in the Experimental Details section also indicates that tumor cells that express insulin receptor protein above a given level are relatively resistant to inhibition by an anti-IGF-1R antibody. Thus, it was found that a sufficiently high value of a gene expression level index comprising the sum of the expression levels of the gene transcripts IR and IR-A is predictive of resistance to growth inhibition by an anti-IGF-1R antibody. For example, tumor cells with an IR plus IR-A gene expression level index at or above that associated with either GEO or SK—N-AS tumor cells were found to be relatively resistant to inhibition by an anti-IGF-1R antibody.

These observations can thus be used to successfully predict which patients will be effectively treated with an IGF-1R kinase inhibitor that inhibits both IGF-1R and IR kinases, such as OSI-906, and of those patients, identify those who would not be effectively treated with an anti-IGF-1R antibody, as an alternative therapy. Thus, these observations can form the basis of valuable new diagnostic methods for predicting the effects of IGF-1R kinase inhibitors on tumor growth, and give oncologists additional tools to assist them in choosing the most appropriate treatment for their patients.

Accordingly, the present invention provides a method of identifying patients with cancer who are likely to benefit from treatment with an IGF-1R kinase inhibitor that inhibits both IGF-1R and IR kinases, comprising: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than the value of the expression level index for RDES tumor cells determined by identical methods, the patient is likely to benefit from treatment with an IGF-1R kinase inhibitor that inhibits both IGF-1R and IR kinases.

The present invention also provides a method of identifying patients with cancer who are likely to benefit from treatment with an IGF-1R kinase inhibitor that inhibits both IGF-1R and IR kinases, comprising: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than the value of the expression level index for SK—N-AS tumor cells determined by identical methods, the patient is likely to benefit from treatment with an IGF-1R kinase inhibitor that inhibits both IGF-1R and IR kinases.

The present invention also provides a method of identifying patients with cancer who are likely to benefit from treatment with an IGF-1R kinase inhibitor that inhibits both IGF-1R and IR kinases, but would likely not respond to therapy with an anti-IGF-1R antibody, comprising: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than that value of the expression level index for RDES tumor cells as determined by identical methods, the patient is likely to benefit from treatment with an IGF-1R kinase inhibitor that inhibits both IGF-1R and IR kinases; determining that if the value of the sum of expression levels for IR and IR-A for the tumor cells of the sample is equal to or greater than the sum of expression levels for IR and IR-A for GEO tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody.

The present invention also provides a method of identifying patients with cancer who are likely to benefit from treatment with an IGF-1R kinase inhibitor that inhibits both IGF-1R and IR kinases, but would likely not respond to therapy with an anti-IGF-1R antibody, comprising: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than that value of the expression level index for SK—N-AS tumor cells as determined by identical methods, the patient is likely to benefit from treatment with an IGF-1R kinase inhibitor that inhibits both IGF-1R and IR kinases; determining that if the value of the sum of expression levels for IR and IR-A for the tumor cells of the sample is equal to or greater than the sum of expression levels for IR and IR-A for GEO tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody.

The present invention also provides a method of identifying patients with cancer who are likely to benefit from treatment with an IGF-1R kinase inhibitor that inhibits both IGF-1R and IR kinases, but would likely not respond to therapy with an anti-IGF-1R antibody, comprising: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than that value of the expression level index for RDES tumor cells as determined by identical methods, the patient is likely to benefit from treatment with an IGF-1R kinase inhibitor that inhibits both IGF-1R and IR kinases; determining that if the value of the sum of expression levels for IR and IR-A for the tumor cells of the sample is equal to or greater than the sum of expression levels for IR and IR-A for A673 tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody.

The present invention also provides a method of identifying patients with cancer who are likely to benefit from treatment with an IGF-1R kinase inhibitor that inhibits both IGF-1R and IR kinases, but would likely not respond to therapy with an anti-IGF-1R antibody, comprising: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than that value of the expression level index for SK—N-AS tumor cells as determined by identical methods, the patient is likely to benefit from treatment with an IGF-1R kinase inhibitor that inhibits both IGF-1R and IR kinases; determining that if the value of the sum of expression levels for IR and IR-A for the tumor cells of the sample is equal to or greater than the sum of expression levels for IR and IR-A for A673 tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody.

The invention also provides a method for treating cancer in a patient, comprising the steps of: (A) diagnosing a patient's likely responsiveness to an IGF-1R kinase inhibitor that inhibits both IGF-1R and IR kinases by determining if the patient has a tumor that is likely to respond to treatment with such an IGF-1R kinase inhibitor by: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than the value of the expression level index for RDES tumor cells determined by identical methods, the patient is likely to benefit from treatment with an IGF-1R kinase inhibitor that inhibits both IGF-1R and IR kinases; and (B) administering to said patient a therapeutically effective amount of an IGF-1R kinase inhibitor that inhibits both IGF-1R and IR kinases if the patient is diagnosed to be potentially responsive to such an IGF-1R kinase inhibitor.

The invention also provides a method for treating cancer in a patient, comprising the steps of: (A) diagnosing a patient's likely responsiveness to an IGF-1R kinase inhibitor that inhibits both IGF-1R and IR kinases by determining if the patient has a tumor that is likely to respond to treatment with such an IGF-1R kinase inhibitor by: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than the value of the expression level index for SK—N-AS tumor cells determined by identical methods, the patient is likely to benefit from treatment with an IGF-1R kinase inhibitor that inhibits both IGF-1R and IR kinases; and (B) administering to said patient a therapeutically effective amount of an IGF-1R kinase inhibitor that inhibits both IGF-1R and IR kinases if the patient is diagnosed to be potentially responsive to such an IGF-1R kinase inhibitor.

The invention also provides a method for treating cancer in a patient, comprising the steps of: (A) diagnosing a patient's likely responsiveness to an IGF-1R kinase inhibitor that inhibits both IGF-1R and IR kinases by determining if the patient has a tumor that is likely to respond to treatment with such an IGF-1R kinase inhibitor, but would likely not respond to therapy with an anti-IGF-1R antibody, by: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than that value of the expression level index for RDES tumor cells as determined by identical methods, the patient is likely to benefit from treatment with an IGF-1R kinase inhibitor that inhibits both IGF-1R and IR kinases; determining that if the value of the sum of expression levels for IR and IR-A for the tumor cells of the sample is equal to or greater than the sum of expression levels for IR and IR-A for GEO tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody; and (B) administering to said patient a therapeutically effective amount of an IGF-1R kinase inhibitor that inhibits both IGF-1R and IR kinases if the patient is diagnosed to be potentially responsive to such an IGF-1R kinase inhibitor.

The invention also provides a method for treating cancer in a patient, comprising the steps of: (A) diagnosing a patient's likely responsiveness to an IGF-1R kinase inhibitor that inhibits both IGF-1R and IR kinases by determining if the patient has a tumor that is likely to respond to treatment with such an IGF-1R kinase inhibitor, but would likely not respond to therapy with an anti-IGF-1R antibody, by: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than that value of the expression level index for SK—N-AS tumor cells as determined by identical methods, the patient is likely to benefit from treatment with an IGF-1R kinase inhibitor that inhibits both IGF-1R and IR kinases; determining that if the value of the sum of expression levels for IR and IR-A for the tumor cells of the sample is equal to or greater than the sum of expression levels for IR and IR-A for GEO tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody; and (B) administering to said patient a therapeutically effective amount of an IGF-1R kinase inhibitor that inhibits both IGF-1R and IR kinases if the patient is diagnosed to be potentially responsive to such an IGF-1R kinase inhibitor.

The invention also provides a method for treating cancer in a patient, comprising the steps of: (A) diagnosing a patient's likely responsiveness to an IGF-1R kinase inhibitor that inhibits both IGF-1R and IR kinases by determining if the patient has a tumor that is likely to respond to treatment with such an IGF-1R kinase inhibitor, but would likely not respond to therapy with an anti-IGF-1R antibody, by: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than that value of the expression level index for RDES tumor cells as determined by identical methods, the patient is likely to benefit from treatment with an IGF-1R kinase inhibitor that inhibits both IGF-1R and IR kinases; determining that if the value of the sum of expression levels for IR and IR-A for the tumor cells of the sample is equal to or greater than the sum of expression levels for IR and IR-A for A673 tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody; and (B) administering to said patient a therapeutically effective amount of an IGF-1R kinase inhibitor that inhibits both IGF-1R and IR kinases if the patient is diagnosed to be potentially responsive to such an IGF-1R kinase inhibitor.

The invention also provides a method for treating cancer in a patient, comprising the steps of: (A) diagnosing a patient's likely responsiveness to an IGF-1R kinase inhibitor that inhibits both IGF-1R and IR kinases by determining if the patient has a tumor that is likely to respond to treatment with such an IGF-1R kinase inhibitor, but would likely not respond to therapy with an anti-IGF-1R antibody, by: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than that value of the expression level index for SK—N-AS tumor cells as determined by identical methods, the patient is likely to benefit from treatment with an IGF-1R kinase inhibitor that inhibits both IGF-1R and IR kinases; determining that if the value of the sum of expression levels for IR and IR-A for the tumor cells of the sample is equal to or greater than the sum of expression levels for IR and IR-A for A673 tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody; and (B) administering to said patient a therapeutically effective amount of an IGF-1R kinase inhibitor that inhibits both IGF-1R and IR kinases if the patient is diagnosed to be potentially responsive to such an IGF-1R kinase inhibitor.

The invention also provides a method for treating cancer in a patient, comprising administering to said patient a therapeutically effective amount of an IGF-1R kinase inhibitor that inhibits both IGF-1R and IR kinases, if the patient has been diagnosed to be potentially responsive to such an IGF-1R kinase inhibitor, by assessing the expression level of the five gene transcripts IGF-1R, IR, IR-A, IGF-1 and IGF-2 in the tumor cells of the cancer; determining an expression level index for the five gene transcripts by adding the expression level values for each of the five transcripts; and determining that the value of the expression level index for the tumor cells is equal to or greater than the value of the expression level index for RDES tumor cells or SK—N-AS tumor cells determined by identical methods. This method is thus a method of treatment targeted at a specific patient population previously identified or characterized as having a tumor susceptible to effective treatment with an IGF-1R kinase inhibitor that inhibits both IGF-1R and IR kinases.

The invention also provides a method for treating cancer in a patient, comprising administering to said patient a therapeutically effective amount of an IGF-1R kinase inhibitor that inhibits both IGF-1R and IR kinases, if the patient has been diagnosed to be potentially responsive to such an IGF-1R kinase inhibitor, by assessing the expression level of the five gene transcripts IGF-1R, IR, IR-A, IGF-1 and IGF-2 in the tumor cells of the cancer; determining an expression level index for the five gene transcripts by adding the expression level values for each of the five transcripts; and determining that the value of the expression level index for the tumor cells is equal to or greater than the value of the expression level index for RDES tumor cells or SK—N-AS tumor cells determined by identical methods, and if the patient is diagnosed to be potentially unresponsive to treatment an anti-IGF-1R antibody by determining that the value of the sum of expression levels for IR and IR-A for the tumor cells of the cancer is equal to or greater than the sum of expression levels for IR and IR-A for GEO or A673 tumor cells as determined by identical methods. This method is thus a method of treatment targeted at a specific patient population previously identified or characterized as having a tumor susceptible to effective treatment with an IGF-1R kinase inhibitor that inhibits both IGF-1R and IR kinases.

In addition, the present invention provides a method of predicting the sensitivity of tumor cell growth to inhibition by an IGF-1R kinase inhibitor that inhibits both IGF-1R and IR kinases, comprising: assessing the expression level of the five gene transcripts IGF-1R, IR, IR-A, IGF-1 and IGF-2 in the tumor cells; determining an expression level index for the five gene transcripts by adding the expression level values for each of the five transcripts; determining that if the value of the expression level index for the tumor cells is equal to or greater than the value of the expression level index for RDES tumor cells determined by identical methods, the tumor cells will exhibit high sensitivity to an IGF-1R kinase inhibitor that inhibits both IGF-1R and IR kinases.

In addition, the present invention provides a method of identifying patients with cancer who are likely to benefit from treatment with an IGF-1R kinase inhibitor that inhibits both IGF-1R and IR kinases, but would likely not respond to therapy with an anti-IGF-1R antibody, comprising: assessing the expression level of the five gene transcripts IGF-1R, IR, IR-A, IGF-1 and IGF-2 in tumor cells from a sample of a patient's tumor; determining an expression level index for the five gene transcripts by adding the expression level values for each of the five transcripts; determining that if the value of the expression level index for the tumor cells of the sample is equal to or greater than the value of the expression level index for RDES or SK—N-AS tumor cells as determined by identical methods, the patient is likely to benefit from treatment with an IGF-1R kinase inhibitor that inhibits both IGF-1R and IR kinases; determining that if the value of the sum of expression levels for IR and IR-A for the tumor cells of the sample is equal to or greater than the sum of expression levels for IR and IR-A for GEO or A673 tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody.

The present invention also provides a method of predicting the sensitivity of tumor cell growth to inhibition by an IGF-1R kinase inhibitor that inhibits both IGF-1R and IR kinases, comprising: assessing the expression level of the five gene transcripts IGF-1R, IR, IR-A, IGF-1 and IGF-2 in the tumor cells; determining an expression level index for the five gene transcripts by adding the expression level values for each of the five transcripts; determining that if the value of the expression level index for the tumor cells is equal to or greater than the value of the expression level index for SK—N-AS tumor cells determined by identical methods, the tumor cells will exhibit high sensitivity to an IGF-1R kinase inhibitor that inhibits both IGF-1R and IR kinases.

The present invention also provides a method of identifying tumor cells that would be sensitive to growth inhibition by an IGF-1R kinase inhibitor that inhibits both IGF-1R and IR kinases, but would not be sensitive to inhibition by an anti-IGF-1R antibody, comprising: assessing the expression level of the five gene transcripts IGF-1R, IR, IR-A, IGF-1 and IGF-2 in the tumor cells; determining an expression level index for the five gene transcripts by adding the expression level values for each of the five transcripts; determining that if the value of the expression level index for the tumor cells is equal to or greater than the value of the expression level index for RDES tumor cells determined by identical methods, the tumor cells will exhibit high sensitivity to an IGF-1R kinase inhibitor that inhibits both IGF-1R and IR kinases, and determining that if the value of the sum of expression levels for IR and IR-A for the tumor cells is equal to or greater than the sum of expression levels for IR and IR-A for GEO tumor cells as determined by identical methods, the tumor cells will not be sensitive to inhibition by an anti-IGF-1R antibody.

The present invention also provides a method of identifying tumor cells that would be sensitive to growth inhibition by an IGF-1R kinase inhibitor that inhibits both IGF-1R and IR kinases, but would not be sensitive to inhibition by an anti-IGF-1R antibody, comprising: assessing the expression level of the five gene transcripts IGF-1R, IR, IR-A, IGF-1 and IGF-2 in the tumor cells; determining an expression level index for the five gene transcripts by adding the expression level values for each of the five transcripts; determining that if the value of the expression level index for the tumor cells is equal to or greater than the value of the expression level index for SK—N-AS tumor cells determined by identical methods, the tumor cells will exhibit high sensitivity to an IGF-1R kinase inhibitor that inhibits both IGF-1R and IR kinases, and determining that if the value of the sum of expression levels for IR and IR-A for the tumor cells is equal to or greater than the sum of expression levels for IR and IR-A for GEO tumor cells as determined by identical methods, the tumor cells will not be sensitive to inhibition by an anti-IGF-1R antibody.

The present invention also provides a method of identifying tumor cells that would be sensitive to growth inhibition by an IGF-1R kinase inhibitor that inhibits both IGF-1R and IR kinases, but would not be sensitive to inhibition by an anti-IGF-1R antibody, comprising: assessing the expression level of the five gene transcripts IGF-1R, IR, IR-A, IGF-1 and IGF-2 in the tumor cells; determining an expression level index for the five gene transcripts by adding the expression level values for each of the five transcripts; determining that if the value of the expression level index for the tumor cells is equal to or greater than the value of the expression level index for RDES tumor cells determined by identical methods, the tumor cells will exhibit high sensitivity to an IGF-1R kinase inhibitor that inhibits both IGF-1R and IR kinases, and determining that if the value of the sum of expression levels for IR and IR-A for the tumor cells is equal to or greater than the sum of expression levels for IR and IR-A for A673tumor cells as determined by identical methods, the tumor cells will not be sensitive to inhibition by an anti-IGF-1R antibody.

The present invention also provides a method of identifying tumor cells that would be sensitive to growth inhibition by an IGF-1R kinase inhibitor that inhibits both IGF-1R and IR kinases, but would not be sensitive to inhibition by an anti-IGF-1R antibody, comprising: assessing the expression level of the five gene transcripts IGF-1R, IR, IR-A, IGF-1 and IGF-2 in the tumor cells; determining an expression level index for the five gene transcripts by adding the expression level values for each of the five transcripts; determining that if the value of the expression level index for the tumor cells is equal to or greater than the value of the expression level index for SK—N-AS tumor cells determined by identical methods, the tumor cells will exhibit high sensitivity to an IGF-1R kinase inhibitor that inhibits both IGF-1R and IR kinases, and determining that if the value of the sum of expression levels for IR and IR-A for the tumor cells is equal to or greater than the sum of expression levels for IR and IR-A for A673 tumor cells as determined by identical methods, the tumor cells will not be sensitive to inhibition by an anti-IGF-1R antibody.

Accordingly, the present invention provides a method of identifying patients with cancer who are likely to benefit from treatment with an IGF-1R kinase inhibitor that inhibits both IGF-1R and IR kinases, comprising: obtaining a sample of a patient's tumor, assessing the expression level of the five gene transcripts IGF-1R, IR, IR-A, IGF-1 and IGF-2 in the cells of the sample; determining an expression level index for the five gene transcripts by adding the expression level values for each of the five transcripts; determining that if the value of the expression level index for the cells of the sample is equal to or greater than the value of the expression level index for RDES tumor cells determined by identical methods, the patient is likely to benefit from treatment with an IGF-1R kinase inhibitor that inhibits both IGF-1R and IR kinases.

The present invention also provides a method of identifying patients with cancer who are likely to benefit from treatment with an IGF-1R kinase inhibitor that inhibits both IGF-1R and IR kinases, comprising: obtaining a sample of a patient's tumor, assessing the expression level of the five gene transcripts IGF-1R, IR, IR-A, IGF-1 and IGF-2 in the cells of the sample; determining an expression level index for the five gene transcripts by adding the expression level values for each of the five transcripts; determining that if the value of the expression level index for the cells of the sample is equal to or greater than the value of the expression level index for SK—N-AS tumor cells determined by identical methods, the patient is likely to benefit from treatment with an IGF-1R kinase inhibitor that inhibits both IGF-1R and IR kinases.

The present invention also provides a method of identifying patients with cancer who are likely to benefit from treatment with an IGF-1R kinase inhibitor that inhibits both IGF-1R and IR kinases, but would likely not respond to therapy with an anti-IGF-1R antibody, comprising: obtaining a sample of a patient's tumor, assessing the expression level of the five gene transcripts IGF-1R, IR, IR-A, IGF-1 and IGF-2 in the cells of the sample; determining an expression level index for the five gene transcripts by adding the expression level values for each of the five transcripts; determining that if the value of the expression level index for the cells of the sample is equal to or greater than that value of the expression level index for RDES tumor cells as determined by identical methods, the patient is likely to benefit from treatment with an IGF-1R kinase inhibitor that inhibits both IGF-1R and IR kinases; determining that if the value of the sum of expression levels for IR and IR-A for the cells of the sample is equal to or greater than the sum of expression levels for IR and IR-A for GEO tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody.

The present invention also provides a method of identifying patients with cancer who are likely to benefit from treatment with an IGF-1R kinase inhibitor that inhibits both IGF-1R and IR kinases, but would likely not respond to therapy with an anti-IGF-1R antibody, comprising: obtaining a sample of a patient's tumor, assessing the expression level of the five gene transcripts IGF-1R, IR, IR-A, IGF-1 and IGF-2 in the cells of the sample; determining an expression level index for the five gene transcripts by adding the expression level values for each of the five transcripts; determining that if the value of the expression level index for the cells of the sample is equal to or greater than that value of the expression level index for SK—N-AS tumor cells as determined by identical methods, the patient is likely to benefit from treatment with an IGF-1R kinase inhibitor that inhibits both IGF-1R and IR kinases; determining that if the value of the sum of expression levels for IR and IR-A for the cells of the sample is equal to or greater than the sum of expression levels for IR and IR-A for GEO tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody.

The present invention also provides a method of identifying patients with cancer who are likely to benefit from treatment with an IGF-1R kinase inhibitor that inhibits both IGF-1R and IR kinases, but would likely not respond to therapy with an anti-IGF-1R antibody, comprising: obtaining a sample of a patient's tumor, assessing the expression level of the five gene transcripts IGF-1R, IR, IR-A, IGF-1 and IGF-2 in the cells of the sample; determining an expression level index for the five gene transcripts by adding the expression level values for each of the five transcripts; determining that if the value of the expression level index for the cells of the sample is equal to or greater than that value of the expression level index for RDES tumor cells as determined by identical methods, the patient is likely to benefit from treatment with an IGF-1R kinase inhibitor that inhibits both IGF-1R and IR kinases; determining that if the value of the sum of expression levels for IR and IR-A for the cells of the sample is equal to or greater than the sum of expression levels for IR and IR-A for A673 tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody.

The present invention also provides a method of identifying patients with cancer who are likely to benefit from treatment with an IGF-1R kinase inhibitor that inhibits both IGF-1R and IR kinases, but would likely not respond to therapy with an anti-IGF-1R antibody, comprising: obtaining a sample of a patient's tumor, assessing the expression level of the five gene transcripts IGF-1R, IR, IR-A, IGF-1 and IGF-2 in the cells of the sample; determining an expression level index for the five gene transcripts by adding the expression level values for each of the five transcripts; determining that if the value of the expression level index for the cells of the sample is equal to or greater than that value of the expression level index for SK—N-AS tumor cells as determined by identical methods, the patient is likely to benefit from treatment with an IGF-1R kinase inhibitor that inhibits both IGF-1R and IR kinases; determining that if the value of the sum of expression levels for IR and IR-A for the cells of the sample is equal to or greater than the sum of expression levels for IR and IR-A for A673 tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody.

The invention also provides a method for treating cancer in a patient, comprising the steps of: (A) diagnosing a patient's likely responsiveness to an IGF-1R kinase inhibitor that inhibits both IGF-1R and IR kinases by determining if the patient has a tumor that is likely to respond to treatment with such an IGF-1R kinase inhibitor by: obtaining a sample of a patient's tumor, assessing the expression level of the five gene transcripts IGF-1R, IR, IR-A, IGF-1 and IGF-2 in the cells of the sample; determining an expression level index for the five gene transcripts by adding the expression level values for each of the five transcripts; determining that if the value of the expression level index for the cells of the sample is equal to or greater than the value of the expression level index for RDES tumor cells determined by identical methods, the patient is likely to benefit from treatment with an IGF-1R kinase inhibitor that inhibits both IGF-1R and IR kinases; and (B) administering to said patient a therapeutically effective amount of an IGF-1R kinase inhibitor that inhibits both IGF-1R and IR kinases if the patient is diagnosed to be potentially responsive to such an IGF-1R kinase inhibitor.

The invention also provides a method for treating cancer in a patient, comprising the steps of: (A) diagnosing a patient's likely responsiveness to an IGF-1R kinase inhibitor that inhibits both IGF-1R and IR kinases by determining if the patient has a tumor that is likely to respond to treatment with such an IGF-1R kinase inhibitor by: obtaining a sample of a patient's tumor, assessing the expression level of the five gene transcripts IGF-1R, IR, IR-A, IGF-1 and IGF-2 in the cells of the sample; determining an expression level index for the five gene transcripts by adding the expression level values for each of the five transcripts; determining that if the value of the expression level index for the cells of the sample is equal to or greater than the value of the expression level index for SK—N-AS tumor cells determined by identical methods, the patient is likely to benefit from treatment with an IGF-1R kinase inhibitor that inhibits both IGF-1R and IR kinases; and (B) administering to said patient a therapeutically effective amount of an IGF-1R kinase inhibitor that inhibits both IGF-1R and IR kinases if the patient is diagnosed to be potentially responsive to such an IGF-1R kinase inhibitor.

The invention also provides a method for treating cancer in a patient, comprising the steps of: (A) diagnosing a patient's likely responsiveness to an IGF-1R kinase inhibitor that inhibits both IGF-1R and IR kinases by determining if the patient has a tumor that is likely to respond to treatment with such an IGF-1R kinase inhibitor, but would likely not respond to therapy with an anti-IGF-1R antibody, by: obtaining a sample of a patient's tumor, assessing the expression level of the five gene transcripts IGF-1R, IR, IR-A, IGF-1 and IGF-2 in the cells of the sample; determining an expression level index for the five gene transcripts by adding the expression level values for each of the five transcripts; determining that if the value of the expression level index for the cells of the sample is equal to or greater than that value of the expression level index for RDES tumor cells as determined by identical methods, the patient is likely to benefit from treatment with an IGF-1R kinase inhibitor that inhibits both IGF-1R and IR kinases; determining that if the value of the sum of expression levels for IR and IR-A for the cells of the sample is equal to or greater than the sum of expression levels for IR and IR-A for GEO tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody; and (B) administering to said patient a therapeutically effective amount of an IGF-1R kinase inhibitor that inhibits both IGF-1R and IR kinases if the patient is diagnosed to be potentially responsive to such an IGF-1R kinase inhibitor.

The invention also provides a method for treating cancer in a patient, comprising the steps of: (A) diagnosing a patient's likely responsiveness to an IGF-1R kinase inhibitor that inhibits both IGF-1R and IR kinases by determining if the patient has a tumor that is likely to respond to treatment with such an IGF-1R kinase inhibitor, but would likely not respond to therapy with an anti-IGF-1R antibody, by: obtaining a sample of a patient's tumor, assessing the expression level of the five gene transcripts IGF-1R, IR, IR-A, IGF-1 and IGF-2 in the cells of the sample; determining an expression level index for the five gene transcripts by adding the expression level values for each of the five transcripts; determining that if the value of the expression level index for the cells of the sample is equal to or greater than that value of the expression level index for SK—N-AS tumor cells as determined by identical methods, the patient is likely to benefit from treatment with an IGF-1R kinase inhibitor that inhibits both IGF-1R and IR kinases; determining that if the value of the sum of expression levels for IR and IR-A for the cells of the sample is equal to or greater than the sum of expression levels for IR and IR-A for GEO tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody; and (B) administering to said patient a therapeutically effective amount of an IGF-1R kinase inhibitor that inhibits both IGF-1R and IR kinases if the patient is diagnosed to be potentially responsive to such an IGF-1R kinase inhibitor.

The invention also provides a method for treating cancer in a patient, comprising the steps of: (A) diagnosing a patient's likely responsiveness to an IGF-1R kinase inhibitor that inhibits both IGF-1R and IR kinases by determining if the patient has a tumor that is likely to respond to treatment with such an IGF-1R kinase inhibitor, but would likely not respond to therapy with an anti-IGF-1R antibody, by: obtaining a sample of a patient's tumor, assessing the expression level of the five gene transcripts IGF-1R, IR, IR-A, IGF-1 and IGF-2 in the cells of the sample; determining an expression level index for the five gene transcripts by adding the expression level values for each of the five transcripts; determining that if the value of the expression level index for the cells of the sample is equal to or greater than that value of the expression level index for RDES tumor cells as determined by identical methods, the patient is likely to benefit from treatment with an IGF-1R kinase inhibitor that inhibits both IGF-1R and IR kinases; determining that if the value of the sum of expression levels for IR and IR-A for the cells of the sample is equal to or greater than the sum of expression levels for IR and IR-A for A673 tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody; and (B) administering to said patient a therapeutically effective amount of an IGF-1R kinase inhibitor that inhibits both IGF-1R and IR kinases if the patient is diagnosed to be potentially responsive to such an IGF-1R kinase inhibitor.

The invention also provides a method for treating cancer in a patient, comprising the steps of:
(A) diagnosing a patient's likely responsiveness to an IGF-1R kinase inhibitor that inhibits both IGF-1R and IR kinases by determining if the patient has a tumor that is likely to respond to treatment with such an IGF-1R kinase inhibitor, but would likely not respond to therapy with an anti-IGF-1R antibody, by: obtaining a sample of a patient's tumor, assessing the expression level of the five gene transcripts IGF-1R, IR, IR-A, IGF-1 and IGF-2 in the cells of the sample; determining an expression level index for the five gene transcripts by adding the expression level values for each of the five transcripts; determining that if the value of the expression level index for the cells of the sample is equal to or greater than that value of the expression level index for SK—N-AS tumor cells as determined by identical methods, the patient is likely to benefit from treatment with an IGF-1R kinase inhibitor that inhibits both IGF-1R and IR kinases; determining that if the value of the sum of expression levels for IR and IR-A for the cells of the sample is equal to or greater than the sum of expression levels for IR and IR-A for A673 tumor cells as determined by identical methods, the patient is not likely to benefit from treatment with an anti-IGF-1R antibody, thus identifying patients with cancer who are likely to benefit from treatment with an IGF-1R kinase inhibitor that inhibits both IGF-1R and IR kinases, but would likely not respond to therapy with an anti-IGF-1R antibody; and
(B) administering to said patient a therapeutically effective amount of an IGF-1R kinase inhibitor that inhibits both IGF-1R and IR kinases if the patient is diagnosed to be potentially responsive to such an IGF-1R kinase inhibitor.

The invention also provides a method for treating a patient with a tumor, comprising administering to said patient a therapeutically effective amount of an IGF-1R kinase inhibitor that inhibits both IGF-1R and IR kinases, if the patient is diagnosed to be potentially responsive to such an IGF-1R kinase inhibitor, by assessing the expression level of the five gene transcripts IGF-1R, IR, IR-A, IGF-1 and IGF-2 in the cells of the tumor; determining an expression level index for the five gene transcripts by adding the expression level values for each of the five transcripts; and determining that the value of the expression level index for the cells of the tumor is equal to or greater than the value of the expression level index for RDES tumor cells or SK—N-AS tumor cells determined by identical methods.

The invention also provides a method for treating a patient with a tumor, comprising administering to said patient a therapeutically effective amount of an IGF-1R kinase inhibitor that inhibits both IGF-1R and IR kinases, if the patient is diagnosed to be potentially responsive to such an IGF-1R kinase inhibitor, by assessing the expression level of the five gene transcripts IGF-1R, IR, IR-A, IGF-1 and IGF-2 in the cells of the tumor; determining an expression level index for the five gene transcripts by adding the expression level values for each of the five transcripts; and determining that the value of the expression level index for the cells of the tumor is equal to or greater than the value of the expression level index for RDES tumor cells or SK—N-AS tumor cells determined by identical methods, and if the patient is diagnosed to be potentially unresponsive to treatment an anti-IGF-1R antibody by determining that the value of the sum of expression levels for IR and IR-A for the cells of the tumor is equal to or greater than the sum of expression levels for IR and IR-A for GEO or A673 tumor cells as determined by identical methods.

In addition, the present invention provides a method of predicting the sensitivity of tumor growth to inhibition by an IGF-1R kinase inhibitor that inhibits both IGF-1R and IR kinases, comprising: assessing the expression level of the five gene transcripts IGF-1R, IR, IR-A, IGF-1 and IGF-2 in the cells of the tumor; determining an expression level index for the five gene transcripts by adding the expression level values for each of the five transcripts; determining that if the value of the expression level index for the cells of the tumor is equal to or greater than the value of the expression level index for RDES tumor cells determined by identical methods, tumor growth will exhibit high sensitivity to an IGF-1R kinase inhibitor that inhibits both IGF-1R and IR kinases.

The present invention also provides a method of predicting the sensitivity of tumor growth to inhibition by an IGF-1R kinase inhibitor that inhibits both IGF-1R and IR kinases, comprising: assessing the expression level of the five gene transcripts IGF-1R, IR, IR-A, IGF-1 and IGF-2 in the cells of the tumor; determining an expression level index for the five gene transcripts by adding the expression level values for each of the five transcripts; determining that if the value of the expression level index for the cells of the tumor is equal to or greater than the value of the expression level index for SK—N-AS tumor cells determined by identical methods, tumor growth will exhibit high sensitivity to an IGF-1R kinase inhibitor that inhibits both IGF-1R and IR kinases.

The present invention also provides a method of identifying tumors that would be sensitive to growth inhibition by an IGF-1R kinase inhibitor that inhibits both IGF-1R and IR kinases, but would not be sensitive to inhibition by an anti-IGF-1R antibody, comprising: assessing the expression level of the five gene transcripts IGF-1R, IR, IR-A, IGF-1 and IGF-2 in cells of a tumor; determining an expression level index for the five gene transcripts by adding the expression level values for each of the five transcripts; determining that if the value of the expression level index for the cells of the tumor is equal to or greater than the value of the expression level index for RDES tumor cells determined by identical methods, tumor growth will exhibit high sensitivity to an IGF-1R kinase inhibitor that inhibits both IGF-1R and IR kinases, and determining that if the value of the sum of expression levels for IR and IR-A for the cells of the tumor is equal to or greater than the sum of expression levels for IR and IR-A for GEO tumor cells as determined by identical methods, tumor growth will not be sensitive to inhibition by an anti-IGF-1R antibody.

The present invention also provides a method of identifying tumors that would be sensitive to growth inhibition by an IGF-1R kinase inhibitor that inhibits both IGF-1R and IR kinases, but would not be sensitive to inhibition by an anti-IGF-1R antibody, comprising: assessing the expression level of the five gene transcripts IGF-1R, IR, IR-A, IGF-1 and IGF-2 in the cells of a tumor; determining an expression level index for the five gene transcripts by adding the expression level values for each of the five transcripts; determining that if the value of the expression level index for the cells of the tumor is equal to or greater than the value of the expression level index for SK—N-AS tumor cells determined by identical methods, tumor growth will exhibit high sensitivity to an IGF-1R kinase inhibitor that inhibits both IGF-1R and IR kinases, and determining that if the value of the sum of expression levels for IR and IR-A for the cells of the tumor is equal to or greater than the sum of expression levels for IR and IR-A for GEO tumor cells as determined by identical methods, tumor growth will not be sensitive to inhibition by an anti-IGF-1R antibody.

The present invention also provides a method of identifying tumors that would be sensitive to growth inhibition by an IGF-1R kinase inhibitor that inhibits both IGF-1R and IR kinases, but would not be sensitive to inhibition by an anti-IGF-1R antibody, comprising: assessing the expression level of the five gene transcripts IGF-1R, IR, IR-A, IGF-1 and IGF-2 in the cells of a tumor; determining an expression level index for the five gene transcripts by adding the expression level values for each of the five transcripts; determining that if the value of the expression level index for the cells of the tumor is equal to or greater than the value of the expression level index for RDES tumor cells determined by identical methods, tumor growth will exhibit high sensitivity to an IGF-1R kinase inhibitor that inhibits both IGF-1R and IR kinases, and determining that if the value of the sum of expression levels for IR and IR-A for the cells of the tumor is equal to or greater than the sum of expression levels for IR and IR-A for A673 tumor cells as determined by identical methods, tumor growth will not be sensitive to inhibition by an anti-IGF-1R antibody.

The present invention also provides a method of identifying tumors that would be sensitive to growth inhibition by an IGF-1R kinase inhibitor that inhibits both IGF-1R and IR kinases, but would not be sensitive to inhibition by an anti-IGF-1R antibody, comprising: assessing the expression level of the five gene transcripts IGF-1R, IR, IR-A, IGF-1 and IGF-2 in the cells of a tumor; determining an expression level index for the five gene transcripts by adding the expression level values for each of the five transcripts; determining that if the value of the expression level index for the cells of the tumor is equal to or greater than the value of the expression level index for SK—N-AS tumor cells determined by identical methods, tumor growth will exhibit high sensitivity to an IGF-1R kinase inhibitor that inhibits both IGF-1R and IR kinases, and determining that if the value of the sum of expression levels for IR and IR-A for the cells of the tumor is equal to or greater than the sum of expression levels for IR and IR-A for A673 tumor cells as determined by identical methods, tumor growth will not be sensitive to inhibition by an anti-IGF-1R antibody.

The present invention also provides a method of identifying patients with cancer in need of treatment with an IGF-1R kinase inhibitor who would likely not respond to therapy with an anti-IGF-1R antibody, comprising: obtaining a sample of a patient's tumor, assessing the expression level of the two gene transcripts IR and IR-A in the tumor cells of the sample; and determining that if the value of the sum of expression levels for IR and IR-A for the tumor cells of the sample is equal to or greater than the sum of expression levels for IR and IR-A for GEO or A673 tumor cells as determined by identical methods, the patient is not likely to benefit from treatment with an anti-IGF-1R antibody. The present invention also provides this method where instead of assessing the levels of the two gene transcripts IR and IR-A, the levels of the two proteins encoded by these transcripts are assessed, i.e. IR-B and IR-A proteins (e.g. by immunohistochemical (IHC) analysis).

The present invention also provides a method of identifying patients with cancer in need of treatment with an IGF-1R kinase inhibitor who would likely not respond to therapy with an anti-IGF-1R antibody, comprising: obtaining a sample of a patient's tumor, assessing the level of phospho-IR in the tumor cells of the sample; and determining that if the level of phospho-IR in the tumor cells of the sample is equal to or greater than the level of phospho-IR for GEO or A673 tumor cells as determined by identical methods, the patient is not likely to benefit from treatment with an anti-IGF-1R antibody.

The present invention also provides a method of identifying tumor cells that would not be sensitive to inhibition by an anti-IGF-1R antibody, comprising: assessing the expression level of the two gene transcripts IR and IR-A in the tumor cells; and determining that if the value of the sum of expression levels for IR and IR-A for the tumor cells is equal to or greater than the sum of expression levels for IR and IR-A for GEO or A673 tumor cells as determined by identical methods, the tumor cells will not be sensitive to inhibition by an anti-IGF-1R antibody. The present invention also provides this method where instead of assessing the levels of the two gene transcripts IR and IR-A, the levels of the two proteins encoded by these transcripts are assessed, i.e. IR-B and IR-A proteins (e.g. by immunohistochemical (IHC) analysis).

The present invention also provides a method of identifying tumor cells that would not be sensitive to inhibition by an anti-IGF-1R antibody, comprising: assessing the level of phospho-IR in the tumor cells; and determining that if the level of phospho-IR in the tumor cells is equal to or greater than the level of phospho-IR for GEO or A673 tumor cells as determined by identical methods, the tumor cells will not be sensitive to inhibition by an anti-IGF-1R antibody.

The present invention also provides a method of identifying patients with cancer in need of treatment with an IGF-1R kinase inhibitor who would likely respond to therapy with an anti-IGF-1R antibody, comprising: obtaining a sample of a patient's tumor, assessing the expression level of the two gene transcripts IR and IR-A in the tumor cells of the sample; and determining that if the value of the sum of expression levels for IR and IR-A for the tumor cells of the sample is equal to or less than the sum of expression levels for IR and IR-A for SK—N-AS tumor cells as determined by identical methods, the patient is likely to benefit from treatment with an anti-IGF-1R antibody. In one embodiment of this method the sum of expression levels for IR and IR-A for the tumor cells of the sample is zero or undetectable. The present invention also provides this method where instead of assessing the levels of the two gene transcripts IR and IR-A, the levels of the two proteins encoded by these transcripts are assessed, i.e. IR-B and IR-A proteins (e.g. by immunohistochemical (IHC) analysis).

The present invention also provides a method of identifying patients with cancer in need of treatment with an IGF-1R kinase inhibitor who would likely respond to therapy with an anti-IGF-1R antibody, comprising: obtaining a sample of a patient's tumor, assessing the level of phospho-IR in the tumor cells of the sample; and determining that if the level of phospho-IR in the tumor cells of the sample is equal to or less than the level of phospho-IR for SK—N-AS tumor cells as determined by identical methods, the patient is likely to benefit from treatment with an anti-IGF-1R antibody. In one embodiment of this method the level of phospho-IR in the tumor cells of the sample is zero or undetectable.

The present invention also provides a method of identifying tumor cells that would be sensitive to inhibition by an anti-IGF-1R antibody, comprising: assessing the expression level of the two gene transcripts IR and IR-A in the tumor cells; and determining that if the value of the sum of expression levels for IR and IR-A for the tumor cells is equal to or less than the sum of expression levels for IR and IR-A for SK—N-AS tumor cells as determined by identical methods, the tumor cells will be sensitive to inhibition by an anti-IGF-1R antibody. In one embodiment of this method the sum of expression levels for IR and IR-A for the tumor cells of the sample is zero or undetectable. The present invention also provides this method where instead of assessing the levels of the two gene transcripts IR and IR-A, the levels of the two proteins encoded by these transcripts are assessed, i.e. IR-B and IR-A proteins (e.g. by immunohistochemical (IHC) analysis).

The present invention also provides a method of identifying tumor cells that would be sensitive to inhibition by an anti-IGF-1R antibody, comprising: assessing the level of phospho-IR in the tumor cells; and determining that if the level of phospho-IR in the tumor cells is equal to or less than the level of phospho-IR for SK—N-AS tumor cells as determined by identical methods, the tumor cells will be sensitive to inhibition by an anti-IGF-1R antibody. In one embodiment of this method the level of phospho-IR in the tumor cells of the sample is zero or undetectable.

The present invention also provides a method for treating cancer in a patient, comprising administering to said patient a therapeutically effective amount of an anti-IGF-1R antibody if the patient is determined to be likely to benefit from treatment with an anti-IGF-1R antibody by determining that the value of the sum of expression levels for IR and IR-A for the tumor cells of the patient's tumor is equal to or less than the sum of expression levels for IR and IR-A for SK—N-AS tumor cells as determined by identical methods. The present invention also provides this method where instead of assessing the levels of the two gene transcripts IR and IR-A, the levels of the two proteins encoded by these transcripts are assessed, i.e. IR-B and IR-A proteins (e.g. by immunohistochemical (IHC) analysis).

The present invention also provides a method for treating cancer in a patient, comprising administering to said patient a therapeutically effective amount of an anti-IGF-1R antibody if the patient is determined to be likely to benefit from treatment with an anti-IGF-1R antibody by determining that level of phospho-IR in the tumor cells of the patient's tumor is equal to or less than the level of phospho-IR for SK—N-AS tumor cells as determined by identical methods.

In the methods of this invention, levels of tyrosine phosphorylated proteins, such as phosphorylated RTKs, for example phospho-IR or phospho-IGF-1R, are determined by any method known to one of skill in the art. In one embodiment an anti-phospho-tyrosine antibody is used to assess levels of tyrosine phosphorylated proteins such as phospho-IR or phosphor-IGF-1R. For example, an HRP-conjugated pan anti-phospho-tyrosine antibody may be used.

The present invention also provides a method of identifying patients with cancer who are likely to benefit from treatment with an IGF-1R kinase inhibitor that inhibits both IGF-1R and IR kinases, comprising: obtaining a sample of a patient's tumor, assessing the expression level of the gene transcripts IGF-1R, IGF-1 and IGF-2 in the tumor cells of the sample; determining that if the tumor cells of the sample express IGF-1R, and if the value of the sum of expression levels for IGF-1 and IGF-2 for the tumor cells of the sample greater than the sum of expression levels for IGF-1 and IGF-2 for RD tumor cells as determined by identical methods, the patient is likely to benefit from treatment with an IGF-1R kinase inhibitor that inhibits both IGF-1R and IR kinases. The present invention also provides this method where instead of assessing the levels of the gene transcript IGF-1R, the level of the protein encoded by this transcript is assessed, i.e. IGF-1R protein (e.g. by immunohistochemical (IHC) analysis).

The present invention also provides a method of identifying patients with cancer who are likely to benefit from treatment with an IGF-1R kinase inhibitor that inhibits both IGF-1R and IR kinases, comprising: obtaining a sample of a patient's tumor, assessing the expression level of the gene transcripts IGF-1R, IR, IR-A, IGF-1 and IGF-2 in the tumor cells of the sample; determining that if the tumor cells of the sample express IGF-1R, express IR and/or IR-A, and if the value of the sum of expression levels for IGF-1 and IGF-2 for the tumor cells of the sample greater than the sum of expression levels for IGF-1 and IGF-2 for RD tumor cells as determined by identical methods, the patient is likely to benefit from treatment with an IGF-1R kinase inhibitor that inhibits both IGF-1R and IR kinases. The present invention also provides this method where instead of assessing the levels of the three gene transcripts IGF-1R, IR and IR-A, the levels of the three proteins encoded by these transcripts are assessed, i.e. IGF-1R, IR-B and IR-A proteins (e.g. by immunohistochemical (IHC) analysis).

The present invention also provides a method of predicting the sensitivity of tumor cell growth to inhibition by an IGF-1R kinase inhibitor that inhibits both IGF-1R and IR kinases, comprising: assessing the expression level of the gene transcripts IGF-1R, IGF-1 and IGF-2 in the tumor cells; determining that if the tumor cells express IGF-1R, and if the value of the sum of expression levels for IGF-1 and IGF-2 for the tumor cells is greater than the sum of expression levels for IGF-1 and IGF-2 for RD tumor cells as determined by identical methods, the tumor cells will exhibit high sensitivity to an IGF-1R kinase inhibitor that inhibits both IGF-1R and IR kinases. The present invention also provides this method where instead of assessing the levels of the gene transcript IGF-1R, the level of the three protein encoded by this transcript is assessed, i.e. IGF-1R protein (e.g. by immunohistochemical (IHC) analysis).

The present invention also provides a method of predicting the sensitivity of tumor cell growth to inhibition by an IGF-1R kinase inhibitor that inhibits both IGF-1R and IR kinases, comprising: assessing the expression level of the gene transcripts IGF-1R, IR, IR-A, IGF-1 and IGF-2 in the tumor cells; determining that if the tumor cells express IGF-1R, express IR and/or IR-A, and if the value of the sum of expression levels for IGF-1 and IGF-2 for the tumor cells is greater than the sum of expression levels for IGF-1 and IGF-2 for RD tumor cells as determined by identical methods, the tumor cells will exhibit high sensitivity to an IGF-1R kinase inhibitor that inhibits both IGF-1R and IR kinases. The present invention also provides this method where instead of assessing the levels of the three gene transcripts IGF-1R, IR and IR-A, the levels of the three proteins encoded by these transcripts are assessed, i.e. IGF-1R, IR-B and IR-A proteins (e.g. by immunohistochemical (IHC) analysis).

The present invention also provides a method for treating cancer in a patient, comprising administering to said patient a therapeutically effective amount of an IGF-1R kinase inhibitor that inhibits both IGF-1R and IR kinases if the patient is determined to be likely to benefit from treatment with such an inhibitor by assessing the expression level of the gene transcripts IGF-1R, IGF-1 and IGF-2 in the tumor cells of the patient's tumor; and determining that the tumor cells of the patient's tumor express IGF-1R, and the value of the sum of expression levels for IGF-1 and IGF-2 for the tumor cells of the patient's tumor is greater than the sum of expression levels for IGF-1 and IGF-2 for RD tumor cells as determined by identical methods. The present invention also provides this method where instead of assessing the levels of the gene transcript IGF-1R, the level of the three protein encoded by this transcript is assessed, i.e. IGF-1R protein (e.g. by immunohistochemical (IHC) analysis).

The present invention also provides a method for treating cancer in a patient, comprising administering to said patient a therapeutically effective amount of an IGF-1R kinase inhibitor that inhibits both IGF-1R and IR kinases if the patient has been determined to be likely to benefit from treatment with such an inhibitor by assessing the expression level of the gene transcripts IGF-1R, IR, IR-A, IGF-1 and IGF-2 in the tumor cells of the patient's tumor; and determining that the tumor cells of the patient's tumor express IGF-1R, express IR and/or IR-A, and the value of the sum of expression levels for IGF-1 and IGF-2 for the tumor cells of the patient's tumor is greater than the sum of expression levels for IGF-1 and IGF-2 for RD tumor cells as determined by identical methods. The present invention also provides this method where instead of assessing the levels of the three gene transcripts IGF-1R, IR and IR-A, the levels of the three proteins encoded by these transcripts are assessed, i.e. IGF-1R, IR-B and IR-A proteins (e.g. by immunohistochemical (IHC) analysis). This method is thus a method of treatment targeted at a specific patient population previously identified or characterized as having a tumor susceptible to effective treatment with an IGF-1R kinase inhibitor that inhibits both IGF-1R and IR kinases.

The present invention also provides a method of identifying a patient with a carcinoma who is likely to benefit from treatment with an IGF-1R kinase inhibitor that inhibits both IGF-1R and IR kinases, comprising: obtaining a sample of a patient's tumor, assessing the expression level of the gene transcripts IGF-1R and IGF-2 in the tumor cells of the sample; determining that if the tumor cells of the sample express IGF-1R, and if the expression level of IGF-2 for the tumor cells of the sample is greater than the expression level of IGF-2 for MDAH-2774 tumor cells as determined by identical methods, the patient is likely to benefit from treatment with an IGF-1R kinase inhibitor that inhibits both IGF-1R and IR kinases. The present invention also provides this method where instead of assessing the levels of the gene transcript IGF-1R, the level of the three protein encoded by this transcript is assessed, i.e. IGF-1R protein (e.g. by immunohistochemical (IHC) analysis).

The present invention also provides a method of identifying a patient with a myeloma who is likely to benefit from treatment with an IGF-1R kinase inhibitor that inhibits both IGF-1R and IR kinases, comprising: obtaining a sample of a patient's tumor, assessing the expression level of the gene transcripts IGF-1R and IGF-1 in the tumor cells of the sample; determining that if the tumor cells of the sample express IGF-1R, and if the expression level of IGF-1 for the tumor cells of the sample is greater than the expression level of IGF-1 for U266 tumor cells as determined by identical methods, the patient is likely to benefit from treatment with an IGF-1R kinase inhibitor that inhibits both IGF-1R and IR kinases. The present invention also provides this method where instead of assessing the levels of the gene transcript IGF-1R, the level of the three protein encoded by this transcript is assessed, i.e. IGF-1R protein (e.g. by immunohistochemical (IHC) analysis).

The present invention also provides a method of identifying a patient with a sarcoma who is likely to benefit from treatment with an IGF-1R kinase inhibitor that inhibits both IGF-1R and IR kinases, comprising: obtaining a sample of a patient's tumor, assessing the expression level of the gene transcripts IGF-1R and IGF-1 in the tumor cells of the sample; determining that if the tumor cells of the sample express IGF-1R, and if the expression level of IGF-1 for the tumor cells of the sample is greater than the expression level of IGF-1 for A673 tumor cells as determined by identical methods, the patient is likely to benefit from treatment with an IGF-1R kinase inhibitor that inhibits both IGF-1R and IR kinases. The present invention also provides this method where instead of assessing the levels of the gene transcript IGF-1R, the level of the three protein encoded by this transcript is assessed, i.e. IGF-1R protein (e.g. by immunohistochemical (IHC) analysis).

The present invention also provides a method of identifying patients with cancer who are likely to benefit from treatment with an IGF-1R kinase inhibitor that inhibits both IGF-1R and IR kinases, comprising: obtaining a sample of a patient's tumor, assessing the expression level of the five gene transcripts IGF-1R, IR, IR-A, IGF-1 and IGF-2 in the tumor cells of the sample; determining an expression level index for the five gene transcripts by adding the expression level values for each of the five transcripts; determining that if the value of the expression level index for the tumor cells of the sample is equal to or greater than a predetermined minimum expression level index value below which tumor cells are resistant to IGF-1R kinase inhibitors that inhibits both IGF-1R and IR kinases, the patient is likely to benefit from treatment with an IGF-1R kinase inhibitor that inhibits both IGF-1R and IR kinases. In one embodiment of this method, the predetermined minimum expression level index is the value of the expression level index for RDES or SK—N-AS tumor cells, determined under identical conditions as used for determining the value of the expression level index for the tumor cells of the patient sample. In another embodiment of this method, in an additional step it is also determined if the value of the sum of expression levels for IR and IR-A for the tumor cells of the sample is equal to or greater than a predetermined minimum level for said sum, above which tumor cells are resistant to inhibition by an anti-IGF-1R antibody, thus indicating whether the patient is also likely to benefit from treatment with an anti-IGF-1R antibody. In an embodiment of this additional step, the predetermined minimum level for said sum is the value of the sum for GEO or A673 tumor cells, determined under identical conditions as used for determining the value of the sum for the tumor cells of the patient sample. The present invention also provides a method of treatment of patients with cancer comprising a step of identifying patients with cancer who are likely to benefit from treatment with an IGF-1R kinase inhibitor that inhibits both IGF-1R and IR kinases, using any of the methods described above, followed by a step of administration of an IGF-1R kinase inhibitor that inhibits both IGF-1R and IR kinases if the patient is identified as being potentially responsive.

The present invention also provides a method of identifying patients with cancer who are likely to benefit from treatment with an IGF-1R kinase inhibitor that inhibits both IGF-1R and IR kinases, comprising: obtaining a sample of a patient's tumor, assessing the expression level of the genes IGF-1R, IR, IGF-1 and IGF-2 in the tumor cells of the sample; determining an expression level index for the genes by adding the expression level values for each of the genes; determining that if the value of the expression level index for the tumor cells of the sample is equal to or greater than a predetermined minimum expression level index value below which tumor cells are resistant to IGF-1R kinase inhibitors that inhibits both IGF-1R and IR kinases, the patient is likely to benefit from treatment with an IGF-1R kinase inhibitor that inhibits both IGF-1R and IR kinases. In one embodiment of this method, the predetermined minimum expression level index is the value of the expression level index for RDES or SK—N-AS tumor cells, determined under identical conditions as used for determining the value of the expression level index for the tumor cells of the patient sample. In another embodiment of this method, in an additional step it is also determined if the value of the sum of expression levels for IR and IR-A for the tumor cells of the sample is equal to or greater than a predetermined minimum level for said sum, above which tumor cells are resistant to inhibition by an anti-IGF-1R antibody, thus indicating whether the patient is also likely to benefit from treatment with an anti-IGF-1R antibody. In an embodiment of this additional step, the predetermined minimum level for said sum is the value of the sum for GEO or A673 tumor cells, determined under identical conditions as used for determining the value of the sum for the tumor cells of the patient sample. In one embodiment of these methods assessing the expression level of the genes IGF-1R, IR, IGF-1 and IGF-2 in tumor cells is by determination of mRNA transcript levels for each of the genes, as described elsewhere herein. In an alternative embodiment of these methods assessing the expression level of the genes IGF-1R, IR, IGF-1 and IGF-2 in tumor cells is by determination of protein levels for each of the genes, e.g. by IHC. The present invention also provides a method of treatment of patients with cancer comprising a step of identifying patients with cancer who are likely to benefit from treatment with an IGF-1R kinase inhibitor that inhibits both IGF-1R and IR kinases, using any of the methods described above, followed by a step of administration of an IGF-1R kinase inhibitor that inhibits both IGF-1R and IR kinases if the patient is identified as being potentially responsive.

The present invention also provides a method of identifying patients with cancer who are likely to benefit from treatment with an IGF-1R kinase inhibitor that inhibits both IGF-1R and IR kinases, but would likely not respond to therapy with an anti-IGF-1R antibody, comprising: obtaining a sample of a patient's tumor, assessing the level of phospho-IR and phospho-IGF-1R in the tumor cells of the sample; and determining that if the tumor cells express both phospho-IR and phosphor-IGF-1R, the patient is likely to benefit from treatment with an IGF-1R kinase inhibitor that inhibits both IGF-1R and IR kinases, but would likely not respond to therapy with an anti-IGF-1R antibody.

A method of treatment of patients with cancer comprising administration of an IGF-1R kinase inhibitor that inhibits both IGF-1R and IR kinases to the patient if they are is identified as being potentially responsive to such an inhibitor, but would likely not respond to therapy with an anti-IGF-1R antibody, by determining that the tumor cells of the patient's tumor express both phospho-IR and phosphor-IGF-1R. In one embodiment, the IGF-1R kinase inhibitor that inhibits both IGF-1R and IR kinases is OSI-906.

The present invention also provides a method for treating cancer in a patient, comprising administering to said patient a therapeutically effective combination of an IGF-1R kinase inhibitor that inhibits both IGF-1R and IR kinases and a chemotherapeutic agent, if the chemotherapeutic agent has been determined to upregulate phosphorylation of both IR and IGF-1R in tumor cells. In one embodiment of this method, the chemotherapeutic agent that has been determined to upregulate phosphorylation of both IR and IGF-1R in tumor cells is doxorubicin.

This invention also encompasses any of the methods of the invention described herein, wherein the step of “obtaining a sample of a patient's tumor” is omitted. In such cases, the step of determining tumor biomarker expression (e.g. gene transcript level for IGF-1R, IR, IR-A, IGF-1 or IGF-2) may for example be performed on a previously processed or prepared tumor sample, e.g. a frozen tumor sample, a fixed tumor preparation, a cell extract, an RNA preparation, a protein preparation, or the like, from which biomarker expression can be assessed, or a biological fluid where the tumor biomarker can be found, as an alternative to the tumor sample itself (e.g. a biopsy).

In the methods of this invention the term “expression level index” means a sum of the expression level values of a number of mRNA transcripts. Thus, for example, one expression level index used in the methods of this invention comprises the sum of the expression level values for the five gene transcripts IGF-1R, IR, IR-A, IGF-1 and IGF-2. A second expression level index used in the methods of this invention comprises the sum of the expression level values for the two gene transcripts IR and IR-A. In a preferred embodiment the expression level values of each of the gene transcripts used in determining the value of an expression level index are determined using the same experimental method.

In the methods of the instant invention involving a step of determining whether a test tumor cell, or tumor cell sample, has a value of an expression level index, or sum of gene expression values for a given group of transcripts, greater than or equal to a value or sum in a specific reference tumor cell (e.g. RDES, SK—N-AS, GEO, A673), it will be appreciated by one of skill in the art that one practicing the method is not constrained by having to always make a direct side-by-side comparison between the test tumor cells and a reference tumor cell. The specific reference tumor cells indicated are merely listed to exemplify the minimum cutoff value of expression level index value above which high sensitivity (or a beneficial effect) is predicted, and may be used, for example, to calibrate an assay system for the determination of transcript levels, after which a ditect comparison to a reference tumor cell is not necessary to practice the method. Other tumor cells with similar expression level index values may be used in place of the tumor cells indicated. It will be appreciated by those of skill in the art that a reference tumor cell sample need not be established for each assay, while the assay is being performed, but rather, a baseline or reference can be established by referring to a form of stored information regarding a previously determined cutoff level to discriminate between sensitive and resistant tumor cells (or patient responders and non-responders). Such a form of stored information can include, for example, but is not limited to, a reference chart, listing or electronic file of population or individual data regarding sensitive and resistant tumors or patients, or any other source of data regarding a cutoff level of expression level index value for tumor cell sensitivity or resistance that is useful for the patient or tumor cell to be evaluated.

In practicing the methods of the invention, use of tumor cells with other expression level index values may also be used as reference tumor cells, or to calibrate a transcript assay system. For example, in the methods of the instant invention, where RDES or SK—N-AS tumor cells are used to indicate a value of an expression level index, any of GEO, H929, 8226, 2650, or H295R tumor cells, each of which has approximately double the value of expression level index (i.e. that using the sum of transcripts IGF-1R, IR, IR-A, IGF-1 and IGF-2), may be used instead. However, since they have double the value of expression level index of RDES or SK—N-AS tumor cells, the step of “determining that if the value of the expression level index for the tumor cells of the sample is equal to or greater than that value of the expression level index for RDES (or SK—N-AS) tumor cells determined by identical methods” is replaced by a step of “determining that if the value of the expression level index for the tumor cells of the sample is equal to or greater than half the value of the expression level index for GEO, H929, 8226, 2650, or H295R tumor cells determined by identical methods”. Many of the other tumor cell lines disclosed herein may be similarly used by incorporating a different multiplier into the method to adjust the three expression level index value to that of RDES or SK—N-AS tumor cells, which indicate a minimum value of expression level index above which tumor cells are sensitive to an IGF-1R kinase inhibitor that inhibits both IGF-1R and IR kinases.

Determination of gene expression transcript levels can be by any method known in the art (e.g. RT-PCR), but the test, or sample, tumor cell determination must be by the identical method as used for any reference tumor cell (e.g. RDES, SK—N-AS, GEO, A673), or that used to calibrate the assay method, in order for a valid comparison to be made between the calculated expression level index value of the test or sample tumor cells, and either a reference tumor cell expression level index value, or an assay standard curve. The resulting gene expression transcript level values may, for example, be in the form of absolute values (e.g. molecules/cell), relative levels (e.g. the transcript level relative to a housekeeping gene transcript level, e.g. GAPDH, β-actin, tubulin, 28S copy number, or the like), or in a normalized form (e.g. in the form of a gene transcript level relative to the 4^{th} (upper) quartile, or median expression value, for a given gene transcript for the tumor cells in which the transcript is measured; or normalized to a given percentile value (e.g. 75^{th} percentile)). For normalization, the test or sample tumor cell may be included in a panel of cells with reference tumor cells, for example having a range of sensitivities to an IGF-1R kinase inhibitor that inhibits both IGF-1R and IR kinases, or the data from the test or sample tumor cell may be analysed with data from such a panel.

The NCBI GeneID numbers listed herein are unique identifiers of the genes described herein from the NCBI Entrez Gene database record (National Center for Biotechnology Information (NCBI), U.S. National Library of Medicine, 8600 Rockville Pike, Building 38A, Bethesda, Md. 20894; Internet address http://www.ncbi.nlm.nih.gov/). Accession numbers of representative mRNAs expressed from the genes are also listed herein.

“RDES tumor cells” as used herein, refers to cells of the cell line RD-ES, available from the American Tissue Culture Collection (ATCC) as HTB-166™, derived from a human Ewing's sarcoma. The cell line was initiated by G. Marshall and M. Kirchen from a primary osseous Ewings sarcoma of the humerus. It shows epithelial morphology.

“SK—N-AS tumor cells” as used herein, refers to cells of the cell line SK—N-AS, available from the American Tissue Culture Collection (ATCC) as CRL2137™, and derived from a human neuroblastoma at a bone marrow metastatic site. It shows epithelial morphology.

“GEO tumor cells” as used herein, refers to cells of the cell line GEO, available from the Roswell Park Cancer Institute (RPCC; Buffalo, N.Y.). GEO was derived from a human colon tumor.

“A673 tumor cells” as used herein, refers to cells of the cell line A-673, available from the American Tissue Culture Collection (ATCC) as CRL1598™, derived from a human rhabdomyosarcoma, and showing polygonal morphology.

“RD tumor cells” as used herein, refers to cells of the cell line RD, available from the American Tissue Culture Collection (ATCC) as CCL136™, derived from a human rhabdomyosarcoma, with a morphology of spindle cells and large multinucleated cells.

“MDAH-2774 tumor cells” as used herein, refers to cells of the ovarian tumor cell line MDAH-2774, derived was derived from ascitic fluid of a 38-year-old patient with metastatic serous cystadenocarcinom (Freedman, R., et al. (1978) Characterization of an ovarian carcinoma cell line, Cancer 42, 2352-2359), and shows carcinoma morphology.

“U266 tumor cells” as used herein, refers to cells of the cell line U266B1 [U266], available from the American Tissue Culture Collection (ATCC) as TIB-196™, derived from a human myeloma, and showing lymphoblast morphology.

In the context of this invention, the sensitivity of tumor cell growth to the IGF-1R kinase inhibitor OSI-906 is defined as high if the tumor cell is inhibited with an EC50 (half-maximal effective concentration) of less than 1 μM, and low (i.e. relatively resistant) if the tumor cell is inhibited with an EC50 of greater than 10 μM. Sensitivies between these values are considered intermediate. With other IGF-1R kinase inhibitors that inhibits both IGF-1R and IR kinases, particularly compounds of Formula I as described herein below, a qualitatively similar result is expected since they inhibit tumor cell growth by inhibiting the same signal transduction pathway, although quantitatively the EC50 values may differ depending on the relative cellular potency of the other inhibitor versus OSI-906. Thus, for example, the sensitivity of tumor cell growth to a more potent IGF-1R kinase inhibitor than OSI-906 would be defined as high when the tumor cell is inhibited with an EC50 that is correspondingly lower. In tumor xenograft studies, using tumor cells of a variety of tumor cell types that all have high sensitivity to OSI-906 in culture in vitro, the tumors are consistently inhibited in vivo with a high pencentage tumor growth inhibition (TGI) (see Experimental section herein). In contast, in similar studies, using tumor cells that have low sensitivity to OSI-906 in culture in vitro, the tumors are inhibited in vivo with only a low pencentage tumor growth inhibition (TGI). These data indicate that sensitivity to IGF-1R kinase inhibitors such as OSI-906 in tumor cell culture is predictive of tumor sensitivity in vivo.

The term EC50 (half maximal effective concentration) refers to the concentration of compound which induces a response halfway between the baseline and maximum for the specified exposure time, and is used as a measure of the compound's potency.

Although the experimental examples provided herein involve the IGF-1R kinase inhibitor, OSI-906, the methods of the present invention are not limited to the prediction of patients or tumors that will respond or not respond to any particular IGF-1R kinase inhibitor, but rather, can be used to predict patient's outcome to any IGF-1R kinase inhibitor that inhibits both IGF-1R and IR kinases. Similarly, the methods of treatment with an IGF-1R kinase inhibitor that inhibits both IGF-1R and IR kinases as described herein may use any of this type of IGF-1R kinase inhibitor. Furthermore, in another embodiment of any of the methods described herein the IGF-1R kinase inhibitor may be an IGF-1R kinase inhibitor approved by a government regulatory authority (e.g. US Food and Drug Administration (FDA); European Medicines Agency; Japanese Ministry of Health, Labour & Welfare; UK Medicines and Healthcare Products Regulatory Agency (MHRA)) (e.g. any of the IGF-1R kinase inhibitors disclosed herein that have been so approved).

In the methods of this invention, an IGF-1R kinase inhibitor that inhibits both IGF-1R and IR kinases may be any IGF-1R kinase inhibitor that inhibits both of these receptor-tyrosine kinases, including pharmacologically acceptable salts or polymorphs thereof. In a preferred embodiment the IGF-1R kinase inhibitor that inhibits both IGF-1R and IR kinases is a small molecule IGF-1R kinase inhibitor. In another embodiment the IGF-1R kinase inhibitor that inhibits both IGF-1R and IR kinases is a small molecule IGF-1R kinase inhibitor that is ATP-competitive at the kinase calalytic site. In one embodiment, the ratio of the inhibitor's IC50 (as determined using an in vitro biochemical kinase assay, e.g. see Mulvihill, M. J. et al. (2008) Bioorganic & Medicinal Chemistry, Volume 16, Issue 3, 1359-1375) for IGF-1R kinase versus IR kinase (i.e. IC50 IGF-1R:IC50 IR) is within the range 1:10 to 10:1. In other embodiments, the ratio of the inhibitor's IC50 for IGF-1R kinase versus IR kinase are within a range selected from 1:5 to 5:1; 1:3 to 3:1; 1:2 to 1:3; 1:2 to 1:5; or 1:2 to 1:10. In an additional embodiment, the IGF-1R kinase inhibitor inhibits both IGF-1R and IR kinases, but has no significant inhibitory activity against any other kinases in an in vitro biochemical assay. Examples of IGF-1R kinase inhibitors that inhibit both IGF-1R and IR kinases include, but are not limited to: OSI-906 (cis-3-[8-amino-1-(2-phenyl-quinolin-7-yl)-imidazo[1,5-a]pyrazin-3-yl[-1-methyl-cyclobutanol); PQIP (cis-3-[3-(4-Methyl-piperazin-1-yl)-cyclobutyl]1-(2-phenyl-quinolin-7-yl)-imidazo[1,5-a]pyrazin-8-ylamine); BMS-554417 (Haluska P, et al. Cancer Res 2006;66(1):362-71); BMS 536924 (Huang, F. et al. (2009) Cancer Res. 69(1):161-170); BMS-754807 (Carboni et al. (2009) Molecular Cancer Therapeutics 8(12)).

In any of the methods, compositions or kits of the invention described herein, the term “small molecule IGF-1R kinase inhibitor” refers to a low molecular weight (i.e. less than 5000 Daltons; preferably less than 1000, and more preferably between 300 and 700 Daltons) organic compound that inhibits IGF-1R kinase by binding to the kinase domain of the enzyme. Examples of such compounds include IGF-1R kinase inhibitors of Formula (I) as described herein. The IGF-1R kinase inhibitor of Formula (I) can be any IGF-1R kinase inhibitor compound encompassed by Formula (I) that inhibits IGF-1R kinase upon administration to a patient. Examples of such inhibitors have been published in US Published Patent Application US 2006/0235031, which is incorporated herein in its entirety, and include OSI-906 (cis-3-[8-amino-1-(2-phenyl-quinolin-7-yl)-imidazo[1,5-a]pyrazin-3-yl]-methyl-cyclobutanol), as used in the experiments described herein.

One of skill in the medical arts, particularly pertaining to the application of diagnostic tests and treatment with therapeutics, will recognize that biological systems are somewhat variable and not always entirely predictable, and thus many good diagnostic tests or therapeutics are occasionally ineffective. Thus, it is ultimately up to the judgement of the attending physician to determine the most appropriate course of treatment for an individual patient, based upon test results, patient condition and history, and his own experience. There may even be occasions, for example, when a physician will choose to treat a patient with an IGF-1R kinase inhibitor even when a tumor is not predicted to be particularly sensitive to IGF-1R kinase inhibitors, based on data from diagnostic tests or from other criteria, particularly if all or most of the other obvious treatment options have failed, or if some synergy is anticipated when given with another treatment. The fact that the IGF-1R kinase inhibitors as a class of compounds are relatively well tolerated compared to many other anti-cancer compounds, such as more traditional chemotherapy or cytotoxic agents used in the treatment of cancer, makes this a more viable option. Also, it should be noted that while the methods disclosed herein predict which patients with tumors are likely to receive the most benefit from an IGF-1R kinase inhibitor that inhibits both IGF-1R and IR kinases, it does not necessarily mean that patients with tumors which do not possess the optimal gene transcript signature will receive no benefit, just that a more modest effect is to be anticipated.

Since diagnostic assays in biological systems are rarely infallible, this invention also provides additional embodiments wherein simultaneous employment of more than one diagnostic method for the determination of susceptibility of tumor cell inhibition to IGF-1R kinase inhibitors is utilized. In such embodiments there is likely to be a lower chance of a false prediction, compared to methods employing just a single method for such determination. All diagnostic methods have potential advantages and disadvantages, and while the preferred method will ultimately depend on individual patient circumstances, the use of multiple diagnostic methods will likely improve one's ability to accurately predict the likely outcome of a therapeutic regimen comprising use of an IGF-1R kinase inhibitor. Therefore, this invention also provides methods for diagnosing or for treating a patient with cancer, comprising the use of two or more diagnostic methods for predicting sensitivity to inhibition by IGF-1R kinase inhibitors, followed in the case of a treatment method by administering to said patient of a therapeutically effective amount of an IGF-1R kinase inhibitor if two or more of the diagnostic methods indicate that the patient is potentially responsive to an IGF-1R kinase inhibitor. One of the diagnostic methods for predicting sensitivity to inhibition by IGF-1R kinase inhibitors may be a method as described herein to predict tumor sensitivity to inhibition by an IGF-1R kinase inhibitor that inhibits both IGF-1R and IR kinases. The other diagnostic method(s) may be any method already known in the art for using biomarkers to predict sensitivity to inhibition by IGF-1R kinase inhibitors, e.g. determination of epithelial or mesenchymal biomarker expression level to assess tumor cell EMT status (e.g. E-cadherin; US 2007/0065858; US 20090092596); biomarkers predicting sensitivity or resistance to IGF-1R kinase inhibitors as described in T. Pitts et al. (2009) EORTC Conference, Boston, Mass., abstract #2141; pERK, pHER3 or HER3 (US 2009/0093488); IGF-1, IGF-2, or other biomarkers reported to predict sensitivity to IGF-1R kinase inhibitors (e.g. see Huang F. H. W., et al. Identification of sensitivity markers for BMS-536924, an inhibitor for insulin-like growth factor-1 receptor. J Clin Oncol ASCO Ann Meet Proc Part I 2007;25:3506).

The gene expression transcript levels assessed for the IGF-1R, IGF-2 and IGF-1 transcripts in the methods of the instant invention includes any mRNA expressed by the three IGF-1R, IGF-2 and IGF-1 genes in a tumor cell, i.e. any mRNA naturally expressed by the tumor cell, including for example, naturally occurring allelic variants, splice variants, etc. Thus, in one embodiment, the transcipts include mRNAs expressed by the human genes IGF-1R (GeneID: 3480, insulin-like growth factor 1 receptor), IGF-1 (GeneID: 3479, insulin-like growth factor 1 (somatomedin C)), and IGF-2 (GeneID: 3481, insulin-like growth factor 2 (somatomedin A)), or mRNAs that hybridize under stringent conditions to the complement of these nucleic acids, wherein the stringent conditions comprise, for example, incubating at 42° C. in a solution comprising 50% formamide, 5×SSC, and 1% SDS and washing at 65° C. in a solution comprising 0.2×SSC and 0.1% SDS. Thus, the “IGF-1 transcript” includes, for example, one or more the following transcripts as described in NCBI databases: Insulin-like growth factor 1 isoform 4 preproprotein transcript NM_{—}000618.3; Insulin-like growth factor 1 isoform 1 transcript NM_{—}001111283.1; Insulin-like growth factor 1 isoform 2 transcript NM_{—}001111284.1; and Insulin-like growth factor 1 isoform 3 transcript NM_{—}001111285.1. The “IGF-2 transcript” includes, for example, one or more the following transcripts as described in NCBI databases: Insulin-like growth factor 1 isoform 1 precursor transcript NM_{—}000612.4; Insulin-like growth factor 1 isoform 1 transcript NM_{—}001007139.4; and Insulin-like growth factor 1 isoform 2 transcript NM_{—}001127598.1. The “IGF-1R transcript” includes, for example, the following transcript as described in NCBI databases: Insulin-like growth factor 1 receptor precursor transcript NM_{—}000875.3.

In the methods of the instant invention, in a preferred embodiment, the gene expression transcripts IR and IR-A, resulting from human insulin receptor (GeneID: 3643; INSR) expression, are as follows: (A) The “IR transcript” refers to transcripts measured with assays that detect IR-B transcripts, i.e. Insulin receptor isoform, long precursor transcripts e.g. transcript NM_{—}000208.2 (IR-B; Exon 11+)), including naturally occurring allelic variants; and (B) The “IR-A transcript” refers to transcripts measured with assays that detect IR-A transcripts, i.e. Insulin receptor isoform short precursor transcripts, e.g. transcript NM_{—}001079817.1 (IR-A; Exon 11-), including naturally occurring allelic variants. Assessment of the levels of transcripts IR and IR-A may be performed, for example, by using a combination of one or more PCR primer pairs selected from the following: PCR primers that specifically detect IR-B (e.g. overlapping exon 10-11 boundary); PCR primers that specifically detect IR-A (e.g. overlapping exon 10-12 boundary); and PCR primers that detect both IR-A and IR-B simultaneously (e.g. overlapping exon 5-6 boundary).

In an alternative embodiment of any of the methods of this invention, where the tumor is present in a non-human patient, the transcripts are animal homologues of the human gene transcripts (e.g. from dog, mouse, rat, rabbit, cat, monkey, ape, etc.).

In the methods of the invention, the level of expression of gene transcripts can be assessed by assessing the amount (e.g. absolute amount or concentration) of the transcript in a tumor cell sample, e.g., a tumor biopsy obtained from a patient, or other patient sample containing tumor cells derived from the tumor (e.g. blood, serum, urine, or other bodily fluids or excretions. Samples of a tumor from a patient may be obtained by procedures such as FNA (fine needle aspiration), or core biopsies, which provide larger amounts of tissue. The cell sample may be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the transcript in the sample. Macrodissection and/or microdisection methods (e.g. Laser Microdissection and Pressure Catapulting (LMPC), for example, using the PALM® Micro Beam microscope (P.A.L.M. Microlaser Technologies AG, Bernried, Germany); SL-Microtest UV laser microdissection system (Molecular Machines & Industries, Glattbrugg, Switzerland)) may be used to enrich the tumor cell population of a tumor sample by removing normal tissue cells or stromal cells (e.g. de Bruin E C. et al. BMC Genomics. 2005 Oct. 14;6:142; Dhal, E. et al. Clinical Cancer Research July 2006 12; 3950; Funel, N. et al. Laboratory Investigation (2008) 88, 773-784, doi:10.1038/labinvest.2008.40, published online 19 May 2008). Primary tumor cell cultures may also be prepared in order to produce a pure tumor cell population.

Expression of a transcripts described in this invention may be assessed by any of a wide variety of well known methods for detecting expression of a transcribed nucleic acid. Non-limiting examples of such methods include nucleic acid hybridization methods, nucleic acid reverse transcription methods, and nucleic acid amplification methods.

In another embodiment, expression of a transcript is assessed by preparing mRNA/cDNA (i.e. a transcribed polynucleotide) from cells in a patient sample, and by hybridizing the mRNA/cDNA with a reference polynucleotide which is a complement of a transcript nucleic acid, or a fragment thereof. cDNA can, optionally, be amplified using any of a variety of polymerase chain reaction methods prior to hybridization with the reference polynucleotide. Expression of transcripts can likewise be detected using quantitative PCR to assess the level of expression of the transcripts.

In a related embodiment, a mixture of transcribed polynucleotides obtained from the sample is contacted with a substrate having fixed thereto a polynucleotide complementary to or homologous with at least a portion (e.g. at least 7, 10, 15, 20, 25, 30, 40, 50, 100, 500, or more nucleotide residues) of a transcript nucleic acid. If polynucleotides complementary to or homologous with are differentially detectable on the substrate (e.g. detectable using different chromophores or fluorophores, or fixed to different selected positions), then the levels of expression of a plurality of transcripts can be assessed simultaneously using a single substrate (e.g. a “gene chip” microarray of polynucleotides fixed at selected positions). When a method of assessing transcript expression is used which involves hybridization of one nucleic acid with another, it is preferred that the hybridization be performed under stringent hybridization conditions.

An exemplary method for detecting the presence or absence of a nucleic acid transcript in a biological sample involves obtaining a biological sample (e.g. a tumor biopsy; a tumor-associated body fluid containing tumor cells) from a test subject and contacting the biological sample with a compound or an agent capable of detecting the polypeptide or nucleic acid (e.g., mRNA, cDNA). The detection methods of the invention can thus be used to detect mRNA, or cDNA, for example, in a biological sample. For example, in vitro techniques for detection of mRNA include Northern hybridizations, in situ hybridizations, polymerase chain reaction (PCR), Quantitative, real-time PCR, in vitro transcription, Northern hybridizations and in situ hybridizations.

Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA transcript of the present invention, or homologous cDNA prepared from the transcript. Other suitable probes for use in the methods of the invention are described herein. Hybridization of an mRNA or cDNA with the probe indicates that the transcript in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of transcripts of the present invention.

An alternative method for determining the level of mRNA transcripts in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the tumor cells prior to detection. In such methods, a cell or tissue sample is prepared/processed using known histological methods. The sample is then immobilized on a support, typically a glass slide, and then contacted with a probe that can hybridize to mRNA transcripts.

As an alternative to making determinations based on the absolute expression level of the transcript, determinations may be based on the normalized expression level of the transcript. Expression levels are normalized, for example, by correcting the absolute expression level of a gene by comparing its expression to the expression of another gene e.g., a housekeeping gene that is constitutively expressed. Suitable genes for normalization include housekeeping genes such as the actin gene, or a tumor cell-specific gene that is expressed at a constant level in the tumor cell type of interest. Such normalization allows the comparison of the expression level in one sample, e.g., a patient sample, to another sample, e.g., a non-tumor sample, a control sample, or between samples from different sources.

The invention also encompasses kits for detecting the presence of a transcript in a biological sample, using any of the methods of the invention. Such kits can be used to determine if a subject is suffering from a tumor that is susceptible to inhibition by an IGF-1R kinase inhibitor that inhibits both IGF-1R and IR kinases. For example, the kit can comprise a labeled compound or agent capable of detecting a nucleic acid in a biological sample and means for determining the amount of the mRNA in the sample (e.g. an oligonucleotide probe which binds to DNA or mRNA encoding the protein, PCR primers). Kits can also include reference or control samples, and instructions for interpreting the results obtained using the kit.

For oligonucleotide-based kits, the kit can comprise, for example: (1) an oligonucleotide, e.g., a detectably labeled oligonucleotide, which hybridizes to a nucleic acid sequence or (2) a pair of primers useful for amplifying a transcript nucleic acid molecule, or cDNA. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

In several embodiment of the present invention, the level of an expressed protein is detected in tumor cells. A preferred agent for detecting proteins of the invention is an antibody capable of binding to such a protein or a fragment thereof, preferably an antibody with a detectable label. Antibodies can be polyclonal, or more preferably, monoclonal. An intact antibody, or a fragment or derivative thereof (e.g., Fab or F(ab′)₂) can be used. The term “labeled”, with regard to an antibody, is intended to encompass direct labeling of the antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody.

Proteins from tumor cells can be isolated prior to detection using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a tumor cell sample contains a protein that binds to a given antibody. Examples of such formats include, but are not limited to, enzyme immunoassay (EIA), radioimmunoassay (RIA), immunohistochemistry (IHC), Western blot analysis and enzyme linked immunoabsorbant assay (ELISA). A skilled artisan can readily adapt known protein/antibody detection methods for use in determining whether tumor cells express a protein of the present invention.

In one format, antibodies, or antibody fragments or derivatives, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from tumor cells can be run on polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

For ELISA assays, specific binding pairs can be of the immune or non-immune type Immune specific binding pairs are exemplified by antigen-antibody systems or hapten/anti-hapten systems. There can be mentioned fluorescein/anti-fluorescein, dinitrophenyl/anti-dinitrophenyl, biotin/anti-biotin, peptide/anti-peptide and the like. The antibody member of the specific binding pair can be produced by customary methods familiar to those skilled in the art. Such methods can involve immunizing an animal with the antigen member of the specific binding pair. If the antigen member of the specific binding pair is not immunogenic, e.g., a hapten, it can be covalently coupled to a carrier protein to render it immunogenic. Non-immune binding pairs include systems wherein the two components share a natural affinity for each other but are not antibodies. Exemplary non-immune pairs are biotin-streptavidin, intrinsic factor-vitamin B₁₂, folic acid-folate binding protein and the like.

A variety of methods are available to covalently label antibodies with members of specific binding pairs. Methods are selected based upon the nature of the member of the specific binding pair, the type of linkage desired, and the tolerance of the antibody to various conjugation chemistries. Biotin can be covalently coupled to antibodies by utilizing commercially available active derivatives. Some of these are biotin-N-hydroxy-succinimide which binds to amine groups on proteins; biotin hydrazide which binds to carbohydrate moieties, aldehydes and carboxyl groups via a carbodiimide coupling; and biotin maleimide and iodoacetyl biotin which bind to sulfhydryl groups. Fluorescein can be coupled to protein amine groups using fluorescein isothiocyanate. Dinitrophenyl groups can be coupled to protein amine groups using 2,4-dinitrobenzene sulfate or 2,4-dinitrofluorobenzene. Other standard methods of conjugation can be employed to couple monoclonal antibodies to a member of a specific binding pair including dialdehyde, carbodiimide coupling, homofunctional crosslinking, and heterobifunctional crosslinking. Carbodiimide coupling is an effective method of coupling carboxyl groups on one substance to amine groups on another. Carbodiimide coupling is facilitated by using the commercially available reagent 1-ethyl-3-(dimethyl-aminopropyl)-carbodiimide (EDAC).

Homobifunctional crosslinkers, including the bifunctional imidoesters and bifunctional N-hydroxysuccinimide esters, are commercially available and are employed for coupling amine groups on one substance to amine groups on another. Heterobifunctional crosslinkers are reagents which possess different functional groups. The most common commercially available heterobifunctional crosslinkers have an amine reactive N-hydroxysuccinimide ester as one functional group, and a sulfhydryl reactive group as the second functional group. The most common sulfhydryl reactive groups are maleimides, pyridyl disulfides and active halogens. One of the functional groups can be a photoactive aryl nitrene, which upon irradiation reacts with a variety of groups.

The detectably-labeled antibody or detectably-labeled member of the specific binding pair is prepared by coupling to a reporter, which can be a radioactive isotope, enzyme, fluorogenic, chemiluminescent or electrochemical materials. Two commonly used radioactive isotopes are ¹²⁵I and ³H. Standard radioactive isotopic labeling procedures include the chloramine T, lactoperoxidase and Bolton-Hunter methods for ¹²⁵I and reductive methylation for ³H. The term “detectably-labeled” refers to a molecule labeled in such a way that it can be readily detected by the intrinsic enzymic activity of the label or by the binding to the label of another component, which can itself be readily detected.

Enzymes suitable for use in this invention include, but are not limited to, horseradish peroxidase, alkaline phosphatase, β-galactosidase, glucose oxidase, luciferases, including firefly and renilla, β-lactamase, urease, green fluorescent protein (GFP) and lysozyme. Enzyme labeling is facilitated by using dialdehyde, carbodiimide coupling, homobifunctional crosslinkers and heterobifunctional crosslinkers as described above for coupling an antibody with a member of a specific binding pair.

The labeling method chosen depends on the functional groups available on the enzyme and the material to be labeled, and the tolerance of both to the conjugation conditions. The labeling method used in the present invention can be one of, but not limited to, any conventional methods currently employed including those described by Engvall and Pearlmann, Immunochemistry 8, 871 (1971), Avrameas and Ternynck, Immunochemistry 8, 1175 (1975), Ishikawa et al., J. Immunoassay 4(3):209-327 (1983) and Jablonski, Anal. Biochem. 148:199 (1985).

Labeling can be accomplished by indirect methods such as using spacers or other members of specific binding pairs. An example of this is the detection of a biotinylated antibody with unlabeled streptavidin and biotinylated enzyme, with streptavidin and biotinylated enzyme being added either sequentially or simultaneously. Thus, according to the present invention, the antibody used to detect can be detectably-labeled directly with a reporter or indirectly with a first member of a specific binding pair. When the antibody is coupled to a first member of a specific binding pair, then detection is effected by reacting the antibody-first member of a specific binding complex with the second member of the binding pair that is labeled or unlabeled as mentioned above.

Moreover, the unlabeled detector antibody can be detected by reacting the unlabeled antibody with a labeled antibody specific for the unlabeled antibody. In this instance “detectably-labeled” as used above is taken to mean containing an epitope by which an antibody specific for the unlabeled antibody can bind. Such an anti-antibody can be labeled directly or indirectly using any of the approaches discussed above. For example, the anti-antibody can be coupled to biotin which is detected by reacting with the streptavidin-horseradish peroxidase system discussed above.

In one embodiment of this invention biotin is utilized. The biotinylated antibody is in turn reacted with streptavidin-horseradish peroxidase complex. Orthophenylenediamine, 4-chloro-naphthol, tetramethylbenzidine (TMB), ABTS, BTS or ASA can be used to effect chromogenic detection.

In one immunoassay format for practicing this invention, a forward sandwich assay is used in which the capture reagent has been immobilized, using conventional techniques, on the surface of a support. Suitable supports used in assays include synthetic polymer supports, such as polypropylene, polystyrene, substituted polystyrene, e.g. aminated or carboxylated polystyrene, polyacrylamides, polyamides, polyvinylchloride, glass beads, agarose, or nitrocellulose.

IHC may be used to localize and quantify tumor proteins in cells of a tissue section, using antibodies specific to the proteins of the invention. In one embodiment, IHC double staining may be used to evaluate the expression of two distinct proteins in the same tumor sample, e.g. using rabbit monoclonal antibodies for dual IHC staining of formalin fixed, paraffin-embedded tissue samples.

The invention also encompasses kits for detecting the presence of a tumor protein in a biological sample. Such kits can be used to determine if a subject is suffering from or is at increased risk of developing a tumor that is less susceptible to inhibition by IGF-1R kinase inhibitors. For example, the kit can comprise a labeled compound or agent capable of detecting a protein in a biological sample and means for determining the amount of the protein in the sample (e.g., an antibody which binds the protein or a fragment thereof). Kits can also include instructions for interpreting the results obtained using the kit. For antibody-based kits, the kit can comprise, for example: (1) a first antibody (e.g., attached to a solid support) which binds to a tumor protein; and, optionally, (2) a second, different antibody which binds to either the protein or the first antibody and is conjugated to a detectable label.

The present invention further provides a method for treating tumors or tumor metastases in a patient, comprising the steps of diagnosing a patient's likely responsiveness to an IGF-1R kinase inhibitor that inhibits both IGF-1R and IR kinases by assessing whether the tumor cells are sensitive to inhibition by an IGF-1R kinase inhibitor that inhibits both IGF-1R and IR kinases, by for example any of the methods described herein, identifying the patient as one who is likely to demonstrate an effective response to treatment with an IGF-1R kinase inhibitor that inhibits both IGF-1R and IR kinases, and administering to said patient a therapeutically effective amount of an IGF-1R kinase inhibitor that inhibits both IGF-1R and IR kinases. In one embodiment the IGF-1R kinase inhibitor used for treatment comprises OSI-906.

It will be appreciated by one of skill in the medical arts that the exact manner of administering to said patient of a therapeutically effective amount of an IGF-1R kinase inhibitor following a diagnosis of a patient's likely responsiveness to an IGF-1R kinase inhibitor that inhibits both IGF-1R and IR kinases will be at the discretion of the attending physician. The mode of administration, including dosage, combination with other anti-cancer agents, timing and frequency of administration, and the like, may be affected by the diagnosis of a patient's likely responsiveness to an IGF-1R kinase inhibitor, as well as the patient's condition and history. Thus, even patients diagnosed with tumors predicted to be relatively insensitive to IGF-1R kinase inhibitors may still benefit from treatment with such inhibitors, particularly in combination with other anti-cancer agents, or agents that may alter a tumor's sensitivity to IGF-1R kinase inhibitors.

The effectiveness of treatment of any of the methods of treatment described herein can, be determined, for example, by measuring the decrease in size of tumors present in the patients with the neoplastic condition, or by assaying a molecular determinant of the degree of proliferation of the tumor cells.

The present invention further provides any of the methods described herein for treating tumors or tumor metastases, or cancer, in a patient comprising administering to the patient a therapeutically effective amount of an IGF-1R kinase inhibitor that inhibits both IGF-1R and IR kinases, and in addition, simultaneously or sequentially, one or more other cytotoxic, chemotherapeutic or anti-cancer agents, or compounds that enhance the effects of such agents. In the context of this invention, other anti-cancer agents includes, for example, other cytotoxic, chemotherapeutic or anti-cancer agents, or compounds that enhance the effects of such agents, anti-hormonal agents, angiogenesis inhibitors, agents that inhibit or reverse EMT (e.g. TGF-beta receptor inhibitors), tumor cell pro-apoptotic or apoptosis-stimulating agents, histone deacetylase (HDAC) inhibitors, histone demethylase inhibitors, DNA methyltransferase inhibitors, signal transduction inhibitors, anti-proliferative agents, anti-HER2 antibody or an immunotherapeutically active fragment thereof, anti-proliferative agents, “another IGF-1R kinase inhibitor” (i.e. other than the IGF-1R kinase inhibitor of the invention that inhibits both IGF-1R and IR kinases), COX II (cyclooxygenase II) inhibitors, and agents capable of enhancing antitumor immune responses, as described herein.

In the context of this invention, additional other cytotoxic, chemotherapeutic or anti-cancer agents, or compounds that enhance the effects of such agents, include, for example: alkylating agents or agents with an alkylating action, such as cyclophosphamide (CTX; e.g. CYTOXAN®), chlorambucil (CHL; e.g. LEUKERAN®), cisplatin (CisP; e.g. PLATINOL®) busulfan (e.g. MYLERAN®), melphalan, carmustine (BCNU), streptozotocin, triethylenemelamine (TEM), mitomycin C, and the like; anti-metabolites, such as methotrexate (MTX), etoposide (VP16; e.g. VEPESID®), 6-mercaptopurine (6MP), 6-thiocguanine (6TG), cytarabine (Ara-C), 5-fluorouracil (5-FU), capecitabine (e.g.XELODA®), dacarbazine (DTIC), and the like; antibiotics, such as actinomycin D, doxorubicin (DXR; e.g. ADRIAMYCIN®), daunorubicin (daunomycin), bleomycin, mithramycin and the like; alkaloids, such as vinca alkaloids such as vincristine (VCR), vinblastine, and the like; and other antitumor agents, such as paclitaxel (e.g. TAXOL®) and pactitaxel derivatives, the cytostatic agents, glucocorticoids such as dexamethasone (DEX; e.g. DECADRON®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. ETHYOL®), dactinomycin, mechlorethamine (nitrogen mustard), streptozocin, cyclophosphamide, lomustine (CCNU), doxorubicin lipo (e.g. DOXIL®), gemcitabine (e.g. GEMZAR®), daunorubicin lipo (e.g. DAUNOXOME®), procarbazine, mitomycin, docetaxel (e.g. TAXOTERE®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil, and pemetrexed.

The present invention further provides any of the methods described herein for treating tumors or tumor metastases, or cancer, in a patient comprising administering to the patient a therapeutically effective amount of an IGF-1R kinase inhibitor that inhibits both IGF-1R and IR kinases, and in addition, simultaneously or sequentially, one or more other cytotoxic, chemotherapeutic or anti-cancer agents, or compounds that enhance the effects of such agents. In the context of this invention, other anti-cancer agents includes, for example, other cytotoxic, chemotherapeutic or anti-cancer agents, or compounds that enhance the effects of such agents, anti-hormonal agents, angiogenesis inhibitors, agents that inhibit or reverse EMT (e.g. TGF-beta receptor inhibitors), tumor cell pro-apoptotic or apoptosis-stimulating agents, histone deacetylase (HDAC) inhibitors, histone demethylase inhibitors, DNA methyltransferase inhibitors, signal transduction inhibitors, anti-proliferative agents, anti-HER2 antibody or an immunotherapeutically active fragment thereof, anti-proliferative agents, COX II (cyclooxygenase II) inhibitors, and agents capable of enhancing antitumor immune responses, as described herein.

The present invention further provides any of the methods described herein for treating tumors or tumor metastases in a patient comprising administering to the patient a therapeutically effective amount of an IGF-1R kinase inhibitor that inhibits both IGF-1R and IR kinases, and in addition, simultaneously or sequentially, one or more anti-hormonal agents. As used herein, the term “anti-hormonal agent” includes natural or synthetic organic or peptidic compounds that act to regulate or inhibit hormone action on tumors.

Antihormonal agents include, for example: steroid receptor antagonists, anti-estrogens such as tamoxifen, raloxifene, aromatase inhibiting 4(5)-imidazoles, other aromatase inhibitors, 42-hydroxytamoxifen, trioxifene, keoxifene, LY 117018, onapristone, and toremifene (e.g. FARESTON®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (leuteinizing hormone-releasing hormone); the LHRH agonist goserelin acetate, commercially available as ZOLADEX® (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N6-(3-pyridinylcarbonyl)-L-lysyl-N6-(3-pyridinylcarbonyl)-D-lysyl-L-leucyl-N6-(1-methylethyl)-L-lysyl-L-proline (e.g ANTIDE®, Ares-Serono); the LHRH antagonist ganirelix acetate; the steroidal anti-androgens cyproterone acetate (CPA) and megestrol acetate, commercially available as MEGACE® (Bristol-Myers Oncology); the nonsteroidal anti-androgen flutamide (2-methyl-N-[4,20-nitro-3-(trifluoromethyl)phenylpropanamide), commercially available as EULEXIN® (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR, RXR, TR, VDR, and the like.

The use of the cytotoxic and other anticancer agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

The present invention further provides any of the methods described herein for treating tumors or tumor metastases in a patient comprising administering to the patient a therapeutically effective amount of an IGF-1R kinase inhibitor that inhibits both IGF-1R and IR kinases, and in addition, simultaneously or sequentially, one or more angiogenesis inhibitors.

Anti-angiogenic agents include, for example: VEGFR inhibitors, such as SU-5416 and SU-6668 (Sugen Inc. of South San Francisco, Calif., USA), or as described in, for example International Application Nos. WO 99/24440, WO 99/62890, WO 95/21613, WO 99/61422, WO 98/50356, WO 99/10349, WO 97/32856, WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); sunitinib (Pfizer); angiozyme, a synthetic ribozyme from Ribozyme (Boulder, Colo.) and Chiron (Emeryville, Calif.); and antibodies to VEGF, such as bevacizumab (e.g. AVASTIN™, Genentech, South San Francisco, Calif.), a recombinant humanized antibody to VEGF; integrin receptor antagonists and integrin antagonists, such as to αᵥβ₃, αᵥβ₅ and αᵥβ₆ integrins, and subtypes thereof, e.g. cilengitide (EMD 121974), or the anti-integrin antibodies, such as for example αᵥβ₃ specific humanized antibodies (e.g. VITAXIN®); factors such as IFN-alpha (U.S. Pat. Nos. 41530,901, 4,503,035, and 5,231,176); angiostatin and plasminogen fragments (e.g. kringle 1-4, kringle 5, kringle 1-3 (O'Reilly, M. S. et al. (1994) Cell 79:315-328; Cao et al. (1996) J. Biol. Chem. 271: 29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1; Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-4701; suramin and suramin analogs; angiostatic steroids; bFGF antagonists; flk-1 and flt-1 antagonists; anti-angiogenesis agents such as MMP-2 (matrix-metalloproteinase 2) inhibitors and MMP-9 (matrix-metalloproteinase 9) inhibitors. Examples of useful matrix metalloproteinase inhibitors are described in International Patent Publication Nos. WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

The present invention further provides any of the methods described herein for treating tumors or tumor metastases in a patient comprising administering to the patient a therapeutically effective amount of an IGF-1R kinase inhibitor that inhibits both IGF-1R and IR kinases, and in addition, simultaneously or sequentially, one or more tumor cell pro-apoptotic or apoptosis-stimulating agents.

The present invention further provides any of the methods described herein for treating tumors or tumor metastases in a patient comprising administering to the patient a therapeutically effective amount of an IGF-1R kinase inhibitor that inhibits both IGF-1R and IR kinases, and in addition, simultaneously or sequentially, one or more signal transduction inhibitors.

Signal transduction inhibitors include, for example: erbB2 receptor inhibitors, such as organic molecules, or antibodies that bind to the erbB2 receptor, for example, trastuzumab (e.g. HERCEPTIN®); inhibitors of other protein tyrosine-kinases, e.g. imitinib (e.g. GLEEVEC®); EGFR kinase inhibitors (see herein below); Met kinase inhibitors (e.g. PF-2341066); ras inhibitors; raf inhibitors; MEK inhibitors; mTOR inhibitors, including mTOR inhibitors that bind to and directly inhibits both mTORC1 and mTORC2 kinases (e.g. OSI-027, OSI Pharmaceuticals); mTOR inhibitors that are dual PI3K/mTOR kinase inhibitors, such as for example the compound PI-103 as described in Fan, Q-W et al (2006) Cancer Cell 9:341-349 and Knight, Z. A. et al. (2006) Cell 125:733-747; mTOR inhibitors that are dual inhibitors of mTOR kinase and one or more other PIKK (or PIK-related) kinase family members. Such members include MEC1, TEL1, RAD3, MEI-41, DNA-PK, ATM, ATR, TRRAP, PI3K, and PI4K kinases; cyclin dependent kinase inhibitors; protein kinase C inhibitors; PI-3 kinase inhibitors; and PDK-1 inhibitors (see Dancey, J. and Sausville, E. A. (2003) Nature Rev. Drug Discovery 2:92-313, for a description of several examples of such inhibitors, and their use in clinical trials for the treatment of cancer).

EGFR kinase inhibitors include, for example: [6,7-bis(2-methoxyethoxy)-4-quinazolin-4-yl]-(3-ethynylphenyl) amine (also known as OSI-774, erlotinib, or TARCEVA™ (erlotinib HCl); OSI Pharmaceuticals/Genentech/Roche) (U.S. Pat. No. 5,747,498; International Patent Publication No. WO 01/34574, and Moyer, J. D. et al. (1997) Cancer Res. 57:4838-4848); CI-1033 (formerly known as PD183805; Pfizer) (Sherwood et al., 1999, Proc. Am. Assoc. Cancer Res. 40:723); PD-158780 (Pfizer); AG-1478 (University of California); CGP-59326 (Novartis); PKI-166 (Novartis); EKB-569 (Wyeth); GW-2016 (also known as GW-572016 or lapatinib ditosylate; GSK); gefitinib (also known as ZD1839 or IRESSATM; Astrazeneca) (Woodburn et al., 1997, Proc. Am. Assoc. Cancer Res. 38:633); and antibody-based EGFR kinase inhibitors. A particularly preferred low molecular weight EGFR kinase inhibitor that can be used according to the present invention is [6,7-bis(2-methoxyethoxy)-4-quinazolin-4-yl]-(3-ethynylphenyl) amine (i.e. erlotinib), its hydrochloride salt (i.e. erlotinib HCl, TARCEVA™), or other salt forms (e.g. erlotinib mesylate). Antibody-based EGFR kinase inhibitors include any anti-EGFR antibody or antibody fragment that can partially or completely block EGFR activation by its natural ligand. Non-limiting examples of antibody-based EGFR kinase inhibitors include those described in Modjtahedi, H., et al., 1993, Br. J. Cancer 67:247-253; Teramoto, T., et al., 1996, Cancer 77:639-645; Goldstein et al., 1995, Clin. Cancer Res. 1:1311-1318; Huang, S. M., et al., 1999, Cancer Res. 15:59(8):1935-40; and Yang, X., et al., 1999, Cancer Res. 59:1236-1243. Thus, the EGFR kinase inhibitor can be the monoclonal antibody Mab E7.6.3 (Yang, X. D. et al. (1999) Cancer Res. 59:1236-43), or Mab C225 (ATCC Accession No. HB-8508), or an antibody or antibody fragment having the binding specificity thereof. Suitable monoclonal antibody EGFR kinase inhibitors include, but are not limited to, IMC-C225 (also known as cetuximab or ERBITUX™; Imclone Systems), ABX-EGF (Abgenix), EMD 72000 (Merck KgaA, Darmstadt), RH3 (York Medical Bioscience Inc.), and MDX-447 (Medarex/Merck KgaA).

EGFR kinase inhibitors also include, for example multi-kinase inhibitors that have activity on EGFR kinase, i.e. inhibitors that inhibit EGFR kinase and one or more additional kinases. Examples of such compounds include the EGFR and HER2 inhibitor CI-1033 (formerly known as PD183805; Pfizer); the EGFR and HER2 inhibitor GW-2016 (also known as GW-572016 or lapatinib ditosylate; GSK); the EGFR and JAK 2/3 inhibitor AG490 (a tyrphostin); the EGFR and HER2 inhibitor ARRY-334543 (Array BioPharma); BIBW-2992, an irreversible dual EGFR/HER2 kinase inhibitor (Boehringer Ingelheim Corp.); the EGFR and HER2 inhibitor EKB-569 (Wyeth); the VEGF-R2 and EGFR inhibitor ZD6474 (also known as ZACTIMA™; AstraZeneca Pharmaceuticals), and the EGFR and HER2 inhibitor BMS-599626 (Bristol-Myers Squibb).

ErbB2 receptor inhibitors include, for example: ErbB2 receptor inhibitors, such as lapatinib or GW-282974 (both Glaxo Wellcome plc), monoclonal antibodies such as AR-209 (Aronex Pharmaceuticals Inc. of The Woodlands, Tex., USA) and 2B-1 (Chiron), and erbB2 inhibitors such as those described in International Publication Nos. WO 98/02434, WO 99/35146, WO 99/35132, WO 98/02437, WO 97/13760, and WO 95/19970, and U.S. Pat. Nos. 5,587,458, 5,877,305, 6,465,449 and 6,541,481.

The present invention further provides any of the methods described herein for treating tumors or tumor metastases in a patient comprising administering to the patient a therapeutically effective amount of an IGF-1R kinase inhibitor that inhibits both IGF-1R and IR kinases, and in addition, simultaneously or sequentially, an anti-HER2 antibody (e.g. trastuzumab, Genentech) or an immunotherapeutically active fragment thereof.

The present invention further provides any of the methods described herein for treating tumors or tumor metastases in a patient comprising administering to the patient a therapeutically effective amount of an IGF-1R kinase inhibitor that inhibits both IGF-1R and IR kinases, and in addition, simultaneously or sequentially, one or more additional anti-proliferative agents.

Additional antiproliferative agents include, for example. Inhibitors of the enzyme farnesyl protein transferase and inhibitors of the receptor tyrosine kinase PDGFR, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217, and FGFR kinase inhibitors.

Examples of PDGFR kinase inhibitors that can be used according to the present invention include Imatinib (GLEEVEC®; Novartis); SU-12248 (sunitinib malate, SUTENT®; Pfizer); Dasatinib (SPRYCEL®; BMS; also known as BMS-354825); Sorafenib (NEXAVAR®; Bayer; also known as Bay-43-9006); AG-13736 (Axitinib; Pfizer); RPR127963 (Sanofi-Aventis); CP-868596 (Pfizer/OSI Pharmaceuticals); MLN-518 (tandutinib; Millennium Pharmaceuticals); AMG-706 (Motesanib; Amgen); ARAVA® (leflunomide; Sanofi-Aventis; also known as SU101), and OSI-930 (OSI Pharmaceuticals); Additional preferred examples of low molecular weight PDGFR kinase inhibitors that are also FGFR kinase inhibitors that can be used according to the present invention include XL-999 (Exelixis); SU6668 (Pfizer); CHIR-258/TKI-258 (Chiron); RO4383596 (Hoffmann-La Roche) and BIBF-1120 (Boehringer Ingelheim).

Examples of FGFR kinase inhibitors that can be used according to the present invention include RO-4396686 (Hoffmann-La Roche); CHIR-258 (Chiron; also known as TKI-258); PD 173074 (Pfizer); PD 166866 (Pfizer); ENK-834 and ENK-835 (both Enkam Pharmaceuticals A/S); and SU5402 (Pfizer). Additional preferred examples of low molecular weight FGFR kinase inhibitors that are also PDGFR kinase inhibitors that can be used according to the present invention include XL-999 (Exelixis); SU6668 (Pfizer); CHIR-258/TKI-258 (Chiron); RO4383596 (Hoffmann-La Roche), and BIBF-1120 (Boehringer Ingelheim).

The present invention further provides any of the methods described herein for treating tumors or tumor metastases in a patient comprising administering to the patient a therapeutically effective amount of an IGF-1R kinase inhibitor that inhibits both IGF-1R and IR kinases, and in addition, simultaneously or sequentially, a COX II (cyclooxygenase II) inhibitor. Examples of useful COX-II inhibitors include alecoxib (e.g. CELEBREX™), valdecoxib, and rofecoxib.

The present invention further provides any of the methods described herein for treating tumors or tumor metastases in a patient comprising administering to the patient a therapeutically effective amount of an an IGF-1R kinase inhibitor that inhibits both IGF-1R and IR kinases, and in addition, simultaneously or sequentially, treatment with radiation or a radiopharmaceutical.

The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in International Patent Publication WO 99/60023.

The present invention further provides any of the methods described herein for treating tumors or tumor metastases in a patient comprising administering to the patient a therapeutically effective amount of an IGF-1R kinase inhibitor that inhibits both IGF-1R and IR kinases, and in addition, simultaneously or sequentially, treatment with one or more agents capable of enhancing antitumor immune responses.

Agents capable of enhancing antitumor immune responses include, for example: CTLA4 (cytotoxic lymphocyte antigen 4) antibodies (e.g. MDX-CTLA4, ipilimumab, MDX-010), and other agents capable of blocking CTLA4. Specific CTLA4 antibodies that can be used in the present invention include those described in U.S. Pat. No. 6,682,736.

In the context of this invention, an “effective amount” of an agent or therapy is as defined above. A “sub-therapeutic amount” of an agent or therapy is an amount less than the effective amount for that agent or therapy, but when combined with an effective or sub-therapeutic amount of another agent or therapy can produce a result desired by the physician, due to, for example, synergy in the resulting efficacious effects, or reduced side effects.

As used herein, the term “patient” preferably refers to a human in need of treatment with an IGF-1R kinase inhibitor that inhibits both IGF-1R and IR kinases for cancer or a pre-cancerous condition or lesion, including refractory versions of such cancers that have failed to respond to other treatments. However, the term “patient” can also refer to non-human animals, preferably mammals such as dogs, cats, horses, cows, pigs, sheep and non-human primates, among others, that are in need of treatment with an IGF-1R kinase inhibitor that inhibits both IGF-1R and IR kinases.

The cancers, or tumors and tumor metastases, of this invention include, but are not limited to, carcinoma, lymphoma, blastoma, sarcoma, and leukemia or lymphoid malignancies, including NSCL (non-small cell lung), pancreatic, head and neck, oral or nasal squamous cell carcinoma, colon, ovarian or breast cancers, lung cancer, bronchioloalveolar cell lung cancer, bone cancer, skin cancer, cancer of the head or neck, HNSCC, cutaneous or intraocular melanoma, uterine cancer, ovarian cancer, rectal cancer, cancer of the anal region, stomach cancer, gastric cancer, uterine cancer, carcinoma of the fallopian tubes, carcinoma of the endometrium, carcinoma of the vagina, carcinoma of the vulva, Hodgkin's Disease, cancer of the esophagus, cancer of the small intestine, colorectal cancer, cancer of the endocrine system, cancer of the thyroid gland, cancer of the parathyroid gland, cancer of the adrenal gland, adrenocortical carcinoma (ACC), sarcoma of soft tissue, Ewing's sarcoma, rhabdomyosarcoma, myeloma, multiple myeloma, cancer of the urethra, cancer of the penis, prostate cancer, cancer of the bladder, cancer of the ureter, carcinoma of the renal pelvis, mesothelioma, hepatocellular cancer, biliary cancer, cancer of the kidney, renal cell carcinoma, chronic or acute leukemia, lymphocytic lymphomas, neuroblastoma, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. In addition to cancer, the methods of this invention may also be used for precancerous conditions or lesions, including, for example, oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, liver cirrhosis or scarring, and precancerous cervical conditions.

The term “refractory” as used herein is used to define a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease). As used herein the term can apply to any of the treatments or agents described herein, when used as single agents or combinations.

For purposes of the present invention, “co-administration of and “co-administering” an IGF-1R kinase inhibitor that inhibits both IGF-1R and IR kinases with an additional anti-cancer agent (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. The additional agent can be administered prior to, at the same time as, or subsequent to administration of the IGF-1R kinase inhibitor that inhibits both IGF-1R and IR kinases, or in some combination thereof. Where the IGF-1R kinase inhibitor that inhibits both IGF-1R and IR kinases is administered to the patient at repeated intervals, e.g., during a standard course of treatment, the additional agent can be administered prior to, at the same time as, or subsequent to, each administration of the IGF-1R kinase inhibitor that inhibits both IGF-1R and IR kinases, or some combination thereof, or at different intervals in relation to the IGF-1R kinase inhibitor that inhibits both IGF-1R and IR kinases treatment, or in a single dose prior to, at any time during, or subsequent to the course of treatment with the IGF-1R kinase inhibitor that inhibits both IGF-1R and IR kinases.

The IGF-1R kinase inhibitor that inhibits both IGF-1R and IR kinases will typically be administered to the patient in a dose regimen that provides for the most effective treatment of the cancer (from both efficacy and safety perspectives) for which the patient is being treated, as known in the art, and as disclosed, e.g. in International Patent Publication No. WO 01/34574. In conducting the treatment method of the present invention, the IGF-1R kinase inhibitor that inhibits both IGF-1R and IR kinases can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the particular IGF-1R kinase inhibitor being used, and the medical judgement of the prescribing physician as based, e.g., on the results of published clinical studies.

The amount of IGF-1R kinase inhibitor that inhibits both IGF-1R and IR kinases administered and the timing of IGF-1R kinase inhibitor administration will depend on the type (species, gender, age, weight, etc.) and condition of the patient being treated, the severity of the disease or condition being treated, and on the route of administration. For example, a small molecule IGF-1R kinase inhibitor that inhibits both IGF-1R and IR kinasess can be administered to a patient in doses ranging from 0.001 to 100 mg/kg of body weight per day or per week in single or divided doses, or by continuous infusion (see for example, International Patent Publication No. WO 01/34574). In particular, compounds such as OSI-906, or similar compounds, can be administered to a patient in doses ranging from 5-200 mg per day, or 100-1600 mg per week, in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

The IGF-1R kinase inhibitor that inhibits both IGF-1R and IR kinasess and other additional agents can be administered either separately or together by the same or different routes, and in a wide variety of different dosage forms. For example, the IGF-1R kinase inhibitor that inhibits both IGF-1R and IR kinases is preferably administered orally or parenterally. Where the IGF-1R kinase inhibitor is OSI-906, or a similar such compound, oral administration is preferable. Both the IGF-1R kinase inhibitor that inhibits both IGF-1R and IR kinases and other additional agents can be administered in single or multiple doses.

The IGF-1R kinase inhibitor that inhibits both IGF-1R and IR kinases can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored.

The IGF-1R kinase inhibitor that inhibits both IGF-1R and IR kinases can be combined together with various pharmaceutically acceptable inert carriers in the form of sprays, creams, salves, suppositories, jellies, gels, pastes, lotions, ointments, and the like. Administration of such dosage forms can be carried out in single or multiple doses. Carriers include solid diluents or fillers, sterile aqueous media, and various non-toxic organic solvents, etc.

Methods of preparing pharmaceutical compositions comprising an IGF-1R kinase inhibitor that inhibits both IGF-1R and IR kinases are known in the art, and are described, e.g. in International Patent Publication No. WO 01/34574. In view of the teaching of the present invention, methods of preparing pharmaceutical compositions comprising an IGF-1R kinase inhibitor that inhibits both IGF-1R and IR kinases will be apparent from the above-cited publications and from other known references, such as Remington's Pharmaceutical Sciences, Mack Publishing Company, Easton, Pa., 18^{th} edition (1990).

For oral administration of an IGF-1R kinase inhibitor that inhibits both IGF-1R and IR kinases, tablets containing one or both of the active agents are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the IGF-1R kinase inhibitor that inhibits both IGF-1R and IR kinases may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration of either or both of the active agents, solutions in either sesame or peanut oil or in aqueous propylene glycol may be employed, as well as sterile aqueous solutions comprising the active agent or a corresponding water-soluble salt thereof. Such sterile aqueous solutions are preferably suitably buffered, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art.

Additionally, it is possible to topically administer either or both of the active agents, by way of, for example, creams, lotions, jellies, gels, pastes, ointments, salves and the like, in accordance with standard pharmaceutical practice. For example, a topical formulation comprising an IGF-1R kinase inhibitor that inhibits both IGF-1R and IR kinases in about 0.1% (w/v) to about 5% (w/v) concentration can be prepared.

For veterinary purposes, the active agents can be administered separately or together to animals using any of the forms and by any of the routes described above. In a preferred embodiment, the IGF-1R kinase inhibitor that inhibits both IGF-1R and IR kinases is administered in the form of a capsule, bolus, tablet, liquid drench, by injection or as an implant. As an alternative, the IGF-1R kinase inhibitor that inhibits both IGF-1R and IR kinases can be administered with the animal feedstuff, and for this purpose a concentrated feed additive or premix may be prepared for a normal animal feed. Such formulations are prepared in a conventional manner in accordance with standard veterinary practice.

As used herein, the term “another IGF-1R kinase inhibitor”, when referring to an additional IGF-1R kinase inhibitor that is added for combination treatment to the IGF-1R kinase inhibitor of the invention that inhibits both IGF-1R and IR kinases, refers to any IGF-1R kinase inhibitor that is currently known in the art, and includes any chemical entity that, upon administration to a patient, results in inhibition of a biological activity specifically associated with activation of the IGF-1 receptor in the patient, and resulting from the binding to IGF-1R of its natural ligand(s). Such IGF-1R kinase inhibitors include any agent that can block IGF-1R activation and the downstream biological effects of IGF-1R activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the IGF-1 receptor, thereby making the receptor inaccessible to its natural ligand so that its normal biological activity is prevented or reduced. Alternatively, such an inhibitor can act by modulating the dimerization of IGF-1R polypeptides, or interaction of IGF-1R polypeptide with other proteins, or enhance ubiquitination and endocytotic degradation of IGF-1R. An IGF-1R kinase inhibitor can also act by reducing the amount of IGF-1 available to activate IGF-1R, by for example antagonizing the binding of IGF-1 to its receptor, by reducing the level of IGF-1, or by promoting the association of IGF-1 with proteins other than IGF-1R such as IGF binding proteins (e.g. IGFBP3). IGF-1R kinase inhibitors include but are not limited to low molecular weight inhibitors, antibodies or antibody fragments, antisense constructs, small inhibitory RNAs (i.e. RNA interference by dsRNA; RNAi), and ribozymes. In a preferred embodiment, the IGF-1R kinase inhibitor is a small organic molecule or an antibody that binds specifically to the human IGF-1R.

IGF-1R kinase inhibitors include, for example imidazopyrazine IGF-1R kinase inhibitors, quinazoline IGF-1R kinase inhibitors, pyrido-pyrimidine IGF-1R kinase inhibitors, pyrimido-pyrimidine IGF-1R kinase inhibitors, pyrrolo-pyrimidine IGF-1R kinase inhibitors, pyrazolo-pyrimidine IGF-1R kinase inhibitors, phenylamino-pyrimidine IGF-1R kinase inhibitors, oxindole IGF-1R kinase inhibitors, indolocarbazole IGF-1R kinase inhibitors, phthalazine IGF-1R kinase inhibitors, isoflavone IGF-1R kinase inhibitors, quinalone IGF-1R kinase inhibitors, and tyrphostin IGF-1R kinase inhibitors, and all pharmaceutically acceptable salts and solvates of such IGF-1R kinase inhibitors.

Additional examples of IGF-1R kinase inhibitors include those in International Patent Publication No. WO 05/097800, that describes 6,6-bicyclic ring substituted heterobicyclic protein kinase inhibitors, International Patent Publication No. WO 05/037836, that describes imidazopyrazine IGF-1R kinase inhibitors, International Patent Publication Nos. WO 03/018021 and WO 03/018022, that describe pyrimidines for treating IGF-1R related disorders, International Patent Publication Nos. WO 02/102804 and WO 02/102805, that describe cyclolignans and cyclolignans as IGF-1R inhibitors, International Patent Publication No. WO 02/092599, that describes pyrrolopyrimidines for the treatment of a disease which responds to an inhibition of the IGF-1R tyrosine kinase, International Patent Publication No. WO 01/72751, that describes pyrrolopyrimidines as tyrosine kinase inhibitors, and in International Patent Publication No. WO 00/71129, that describes pyrrolotriazine inhibitors of kinases, and in International Patent Publication No. WO 97/28161, that describes pyrrolo [2,3-d]pyrimidines and their use as tyrosine kinase inhibitors, Parrizas, et al., which describes tyrphostins with in vitro and in vivo IGF-1R inhibitory activity (Endocrinology, 138:1427-1433 (1997)), International Patent Publication No. WO 00/35455, that describes heteroaryl-aryl ureas as IGF-1R inhibitors, International Patent Publication No. WO 03/048133, that describes pyrimidine derivatives as modulators of IGF-1R, International Patent Publication No. WO 03/024967, WO 03/035614, WO 03/035615, WO 03/035616, and WO 03/035619, that describe chemical compounds with inhibitory effects towards kinase proteins, International Patent Publication No. WO 03/068265, that describes methods and compositions for treating hyperproliferative conditions, International Patent Publication No. WO 00/17203, that describes pyrrolopyrimidines as protein kinase inhibitors, Japanese Patent Publication No. JP 07/133280, that describes a cephem compound, its production and antimicrobial composition, Albert, A. et al., Journal of the Chemical Society, 11: 1540-1547 (1970), which describes pteridine studies and pteridines unsubstituted in the 4-position, and A. Albert et al., Chem. Biol. Pteridines Proc. Int. Symp., 4th, 4: 1-5 (1969) which describes a synthesis of pteridines (unsubstituted in the 4-position) from pyrazines, via 3-4-dihydropteridines.

IGF-1R kinase inhibitors that inhibits both IGF-1R and IR kinasess that are useful in this invention include compounds represented by Formula (I) (see below), as described in US Published Patent Application US 2006/0235031, where their preparation is described in detail. PQIP (cis-3-[3-(4-Methyl-piperazin-1-yl)-cyclobutyl]1-(2-phenyl-quinolin-7-yl)-imidazo[1,5-a]pyrazin-8-ylamine) and OSI-906 (cis-3-[8-amino-1-(2-phenyl-quinolin-7-yl)-imidazo[1,5-a]pyrazin-3-yl]-1-methyl-cyclobutanol) represents IGF-1R kinase inhibitors according to Formula (I).

OSI-906 has the structure as follows:

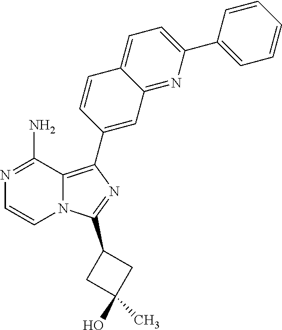

PQIP has the structure as follows:

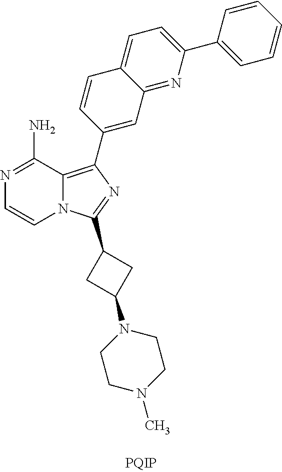

An IGF-1R kinase inhibitor of Formula (I), as described in US Published Patent Application US 2006/0235031, is represented by the formula:

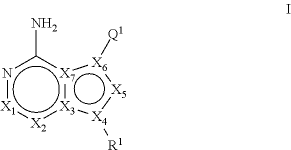

or a pharmaceutically acceptable salt thereof, wherein:

X₁, and X₂ are each independently N or C-(E¹)ₐₐ;

X₅ is N, C-(E¹)ₐₐ, or N-(E¹)ₐₐ;

X₃, X₄, X₆, and X₇ are each independently N or C;
wherein at least one of X₃, X₄, X₅, X₆, and X₇ is independently N or N-(E¹)ₐₐ;

Q¹ is

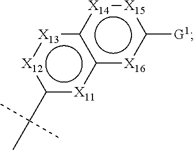

X₁₁, X₁₂, X₁₃, X₁₄, X₁₅, and X₁₆ are each independently N, C-(E¹¹)_{bb}, or N⁺—O⁻;

wherein at least one of X₁₁, X₁₂, X₁₃, X₁₄, X₁₅, and X₁₆ is N or N⁺—O⁻;

R¹ is absent, C₀₋₁₀alkyl, cycloC₃₋₁₀alkyl, bicycloC₅₋₁₀alkyl, aryl, heteroaryl, aralkyl, heteroaralkyl, heterocyclyl, heterobicycloC₅₋₁₀alkyl, spiroalkyl, or heterospiroalkyl, any of which is optionally substituted by one or more independent G¹¹ substituents;

E¹, E¹¹, G¹, and G⁴¹ are each independently halo, —CF₃, —OCF₃, —OR², —NR²R³(R^{2a})ⱼ₁, —C(═O)R², —CO₂R², —CONR²R³, —NO₂, —CN, —S(O)ⱼ₁R², —SO₂NR²R³, —NR²C(═O)R³, —NR²C(═O)OR³, —NR²C(═O)NR³R^{2a}, —NR²S(O)ⱼ₁R³, —C(═S)OR², —C(═O)SR², —NR²C(═NR³)NR^{2a}R^{3a}, —NR²C(═NR³)OR^{2a}, —NR²C(═NR³)SR^{2a}, —OC(═O)OR², —OC(═O)NR²R³, —OC(═O)SR², —SC(═O)OR², —SC(═O)NR²R³, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, oxo, —CF₃, —OCF₃, —OR²²², —NR²²²R³³³(R^{222a})ⱼ₁ₐ, —C(═O)R²²², —CO²R²²², —C(═O)NR²²²R³³³, —NO₂, —CN, —S(═O)ⱼ₁ₐR²²², —SO₂NR²²²R³³³, —NR²²²C(═O)R³³³, —NR²²²C(═O)OR³³³, —NR²²²C(═O)NR³³³R^{222a}, —NR²²²S(O)ⱼ₁ₐR³³³, —C(═S)OR²²², —C(═O)SR²²², —NR²²²C(═NR³³³)NR^{222a}R^{333a}, —NR²²²C(═NR³³³)OR^{222a}, —NR²²²C(═NR³³³)SR^{222a}, —OC(═O)OR²²², —OC(═O)NR²²²R³³³, —OC(═O)SR²²², —SC(═O)OR²²², or —SC(═O)NR²²²R³³³ substituents;

or E¹, E¹¹, or G¹ optionally is —(W¹)ₙ—(Y₁)ₘ—R⁴;

or E¹, E¹¹, G¹, or G⁴¹ optionally independently is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃, —OR₂₂₂, —NR²²²R³³³(R^{222a})ⱼ₂ₐ, —C(O)R²²², —CO₂R²²², —C(═O)NR²²²R³³³, —NO₂, —CN, —S(O)_{J2a}R²²², —SO₂NR²²²R³³³, —NR²²²C(═O)R³³³, —NR²²²C(═O)OR³³³, —NR²²²C(═O)NR³³³R^{222a}, —NR²²²S(═O)ⱼ₂ₐR³³³, —C(═S)OR²²², —C(═O)SR²²², —NR²²²C(═NR³³³)NR^{222a}R^{333a}, —NR²²²C(═NR³³³)OR^{222a}, —NR²²²C(═NR³³³)SR^{222a}, —OC(═O)OR²²², —OC(═O)NR²²²R³³³, —OC(═O)SR²²², —SC(═O)OR²²², or —SC(═O)NR²²²R³³³ substituents;

G¹¹ is halo, oxo, —CF₃, —OCF₃, —OR²¹, —NR²¹R³¹(R^{2a1})ⱼ₄, —C(O)R²¹, —CO₂R²¹, —C(═O)NR²¹R³¹, —NO₂, —CN, —S(O)ⱼ₄R²¹, —SO₂NR²¹R³¹, NR²¹(C═O)R³¹, NR²¹C(═O)OR³¹, NR²¹C(═O)NR³¹R^{2a1}, NR²¹S(O)ⱼ₄R³¹, —C(═S)OR²¹, —C(═O)SR²¹, NR²¹C(═NR³¹)NR^{2a1}R^{3a1}, —NR²¹C(═NR³¹)OR^{2a1}, —NR²¹C(═NR³¹)SR^{2a1}, —OC(═O)OR²¹, —OC(═O)NR²¹R³¹, —OC(═O)SR²¹, —SC(═O)OR²¹, —SC(═O)NR²¹R³¹, —P(O)OR²¹OR³¹, C₁₋₁₀alkylidene, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, oxo, —CF₃, —OCF₃, —OR²²²¹, —NR²²²¹R³³³¹(R^{222a1})ⱼ₄ₐ, —C(O)R²²²¹, —CO₂R²²²¹, —C(═O)NR²²²¹R³³³¹, —NO₂, —CN, —S(O)ⱼ₄ₐR²²²¹, —SO₂NR²²²¹R³³³¹, —NR²²²¹C(═O)R³³³¹, —NR²²²¹C(═O)OR³³³¹, —NR²²²¹C(═O)NR³³³¹R^{222a1}, —NR²²²¹S(O)ⱼ₄ₐR³³³¹, —C(═S)OR²²²¹, —C(═O)SR²²²¹, —NR²²²¹C(═O)OR³³³¹, —NR²²²¹C(═O)NR³³³¹R^{222a1}, —NR²²²¹S(O)ⱼ₄ₐR³³³¹, —C(═S)OR²²²¹, —C(═O)SR²²²¹, —NR²²²¹ C(═NR³³³¹)NR^{222a1}R^{333a1}, —NR²²²¹C(═NR³³³¹)OR^{222a1}, —NR²²²¹C(═NR³³³¹)SR^{222a1}, —OC(═O)OR²²²¹, —OC(═O)NR²²²¹R³³³¹, —OC(═O)SR²²²¹, —SC(═O)OR²²²¹, —P(O)OR²²²¹OR³³³¹, or —SC(═O)NR²²²¹R³³³¹ substituents;

or G¹¹ is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃, —OR²²²¹, —NR²²²¹R³³³¹(R^{222a1})ⱼ₅ₐ, —C(O)R^{—C(═O)NR}²²²¹R³³³¹, —NO₂, —CN, —S(O)ⱼ₅ₐR²²²¹, —SO₂NR²²²¹R³³³¹, —NR²²²¹, —CO₂R²²²¹, —NR²²²¹, C(═O)OR³³³¹, —NR²²²¹C(═O)NR³³³¹R^{222a1}, —NR²²²¹S(O)ⱼ₅ₐR³³³¹, —C(═S)OR²²²¹, —C(═O)SR²²²¹, —OC(═O)OR²²²¹, —OC(═O)NR²²²¹R³³³¹, —OC(═O)SR²²²¹, —SC(═O)OR²²²¹, —P(O)OR²²²¹OR³³³¹, or —SC(═O)NR²²²¹R³³³¹ substituets;

or G¹¹ is C, taken together with the carbon to which it is attached forms a C═C double bond which is substituted with R⁵ and G¹¹¹;

R², R^{2a}, R³, R^{3a}, R²²², R^{222a}, R³³³, R^{333a}, R²¹, R^{2a1}, R³¹, R^{3a1}, R²²²¹, R^{222a1}, R³³³¹, and R^{333a1} are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, or aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted by one or more independent G¹¹¹ substituents;

or in the case of —NR²R³(R^{2a})ⱼ₁ or —NR²²²R³³³(R^{222a})ⱼ₁ₐ or —NR²²²R³³³(R^{222a})ⱼ₂ₐ or —NR²¹R³¹(R^{2a1})ⱼ₄ or —NR²²²¹R³³³¹(R^{222a1})ⱼ₄ₐ or —NR²²²¹R³³³¹(R^{222a1})ⱼ₅ₐ, then R² and R³, or R²²² and R³³³, or R²²²¹ and R³³³¹, respectfully, are optionally taken together with the nitrogen atom to which they are attached to form a 3-10 membered saturated or unsaturated ring, wherein said ring is optionally substituted by one or more independent G¹¹¹¹ substituents and wherein said ring optionally includes one or more heteroatoms other than the nitrogen to which R² and R³, or R²²² and R³³³, or R²²²¹ and R³³³¹ are attached;

W¹ and Y¹ are each independently —O—, —NR⁷—, —S(O)ⱼ₇—, —CR⁵R⁶—, —N(C(O)OR⁷)—, —N(C(O)R⁷)—, —N(SO₂R⁷)—, —CH₂O—, —CH₂S—, —CH₂N(R⁷)—, —CH(NR⁷)—, —CH₂N(C(O)R⁷)—, —CH₂N(C(O)OR⁷)—, —CH₂N(SO₂R⁷)—, —CH(NHR⁷)—, —CH(NHC(O)R⁷)—, —CH(NHSO₂R⁷)—, —CH(NHC(O)OR⁷)—, —CH(OC(O)R⁷)—, —CH(OC(O)NHR⁷)—, —CH═CH—, —C(═NOR⁷)—, —C(O)—, —CH(OR⁷)—, —C(O)N(R⁷)—, —N(R⁷)C(O)—, —N(R⁷)S(O)—, —N(R⁷)S(O)₂——OC(O)N(R⁷)—, —N(R⁷)C(O)N(R⁸)—, —NR⁷C(O)O—, —S(O)N(R⁷)—, —S(O)₂N(R⁷)—, —N(C(O)R⁷)S(O)—, —N(C(O)R⁷)S(O)₂—, —N(R⁷)S(O)N(R⁸)—, —N(R⁷)S(O)₂N(R⁸)—, —C(O)N(R⁷)C(O)—, —S(O)N(R⁷)C(O)—, —S(O)₂N(R⁷)C(O)—, —OS(O)N(R⁷)—, —OS(O)₂N(R⁷)—, —N(R⁷)S(O)O—, —N(R⁷)S(O)₂O—, —N(R⁷)S(O)C(O)—, —N(R⁷)S(O)₂C(O)—, —SON(C(O)R⁷)—, —SO₂N(C(O)R⁷)—, —N(R⁷)SON(R⁸)—, —N(R⁷)SO₂N(R⁸)—, —C(O)O—, —N(R⁷)P(OR⁸)O—, —N(R⁷)P(OR⁸)—, —N(R⁷)P(O)(OR⁸)O—, —N(R⁷)P(O)(OR⁸)—, —N(C(O)R⁷)P(OR⁸)O—, —N(C(O)R⁷)P(OR⁸)—, —N(C(O)R⁷)P(O)(OR⁸)O—, —N(C(O)R⁷)P(OR⁸)—, —CH(R⁷)S(O)—, —CH(R⁷)S(O)₂—, —CH(R⁷)N(C(O)OR⁸)—, —CH(R⁷)N(C(O)R⁸)—, —CH(R⁷)N(SO₂R⁸)—, —CH(R⁷)O—, —CH(R⁷)S—, —CH(R⁷)N(R⁸)—, —CH(R⁷)N(C(O)R⁸)—, —CH(R⁷)N(C(O)OR⁸)—, —CH(R⁷)N(SO₂R⁸)—, —CH(R⁷)C(═NOR⁸)—, —CH(R⁷)C(O)—, —CH(R⁷)CH(OR⁸)—, —CH(R⁷)C(O)N(R⁸)—, —CH(R⁷)N(R⁸)C(O)—, —CH(R⁷)N(R⁸)S(O)—, —CH(R⁷)N(R⁸)S(O)₂—, —CH(R⁷)OC(O)N(R⁸)—, —CH(R⁷)N(R⁸)C(O)N(R^{7a})—, —CH(R⁷)NR⁸C(O)O—, —CH(R⁷)S(O)N(R⁸)—, —CH(R⁷)S(O)₂N(R⁸)—, —CH(R⁷)N(C(O)R⁸)S(O)—, —CH(R⁷)N(C(O)R⁸)S(O)—, —CH(R⁷)N(R⁸)S(O)N(R^{7a})—, —CH(R⁷)N(R⁸)S(O)₂N(R^{7a})—, —CH(R⁷)C(O)N(R⁸)C(O)—, —CH(R⁷)S(O)N(R⁸)C(O)—, —CH(R⁷)S(O)₂N(R⁸)C(O)—, —CH(R⁷)OS(O)N(R⁸)—, —CH(R⁷)OS(O)₂N(R⁸)—, —CH(R⁷)N(R⁸)S(O)O—, —CH(R⁷)N(R⁸)S(O)₂O—, —CH(R⁷)N(R⁸)S(O)C(O)—, —CH(R⁷)N(R⁸)S(O)₂C(O)—, —CH(R⁷)SON(C(O)R⁸)—, —CH(R⁷)SO₂N(C(O)R⁸)—, —CH(R⁷)N(R⁸)SON(R^{7a})—, —CH(R⁷)N(R⁸)SO₂N(R^{7a})—, —CH(R⁷)C(O)O—, —CH(R⁷)N(R⁸)P(OR^{7a})O—, —CH(R⁷)N(R⁸)P(OR^{7a})—, —CH(R⁷)N(R⁸)P(O)(OR^{7a})O—, —CH(R⁷)N(R⁸)P(O)(OR^{7a})—, —CH(R⁷)N(C(O)R⁸)P(OR^{7a})O—, —CH(R⁷)N(C(O)R⁸)P(OR^{7a})—, —CH(R⁷)N(C(O)R⁸)P(O)(OR^{7a})O—, or —CH(R⁷)N(C(O)R⁸)P(OR^{7a})—;

R⁵, R⁶, G¹¹¹, and G¹¹¹¹ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃, —OR⁷⁷, —NR⁷⁷R⁸⁷, —C(O)R⁷⁷, —CO₂ R⁷⁷, —CONR⁷⁷R⁸⁷, —NO₂, —CN, —S(O)ⱼ₅ₐR⁷⁷, —SO₂NR⁷⁷R⁸⁷, —NR⁷⁷C(═O)R⁸⁷, —NR⁷⁷C(═O)OR⁸⁷, —NR⁷⁷C(═O)NR⁷⁸R⁸⁷, —NR⁷⁷S(O)ⱼ₅ₐR⁸⁷, —C(═S)OR⁷⁷, —C(═O)SR⁷⁷, —NR⁷⁷C(═NR⁸⁷)NR⁷⁸R⁸⁸, —NR⁷⁷C(═NR⁸⁷)OR⁷⁸, —NR⁷⁷C(═NR⁸⁷)SR⁷⁸, —OC(═O)OR⁷⁷, —OC(═O)NR⁷⁷R⁸⁷, —OC(═O)SR⁷⁷, —SC(═O)OR⁷⁷, —P(O)OR⁷⁷OR⁸⁷, or —SC(═O)NR⁷⁷R⁸⁷ substituents;

or R⁵ with R⁶ are optionally taken together with the carbon atom to which they are attached to form a 3-10 membered saturated or unsaturated ring, wherein said ring is optionally substituted with one or more independent R⁶⁹ substituents and wherein said ring optionally includes one or more heteroatoms;

R⁷, R^{7a}, and R⁸ are each independently acyl, C₀₋₁₀alkyl, C₂₋₁₀alkenyl, aryl, heteroaryl, heterocyclyl or cycloC₃₋₁₀alkyl, any of which is optionally substituted by one or more independent substituents;

R⁴ is C₀₋₁₀alkyl, C₂₋₁₀alkenyl, C₂₋₁₀alkynyl, aryl, heteroaryl, cycloC₃₋₁₀alkyl, heterocyclyl, cycloC₃₋₈alkenyl, or heterocycloalkenyl, any of which is optionally substituted by one or more independent G⁴¹ substituents;

R⁶⁹ is halo, —OR⁷⁸, —SH, —NR⁷⁸R⁸⁸, —CO₂R⁷⁸, —C(═O)NR⁷⁸R⁸⁸, —NO₂, —CN, —S(O)ⱼ₈R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸, or —NR⁷⁷⁸R⁸⁸⁸ substituents;

or R⁶⁹ is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —C(═O)NR⁷⁷⁸R⁸⁸⁸, —SO₂NR⁷⁷⁸R⁸⁸⁸, or —NR⁷⁷⁸R⁸⁸⁸ substituents;

or in the case of —NR⁷⁸R⁸⁸, R⁷⁸ and R⁸⁸ are optionally taken together with the nitrogen atom to which they are attached to form a 3-10 membered saturated or unsaturated ring, wherein said ring is optionally substituted with one or more independent halo, cyano, hydroxy, nitro, C₁₋₁₀alkoxy, —SO₂NR⁷⁷⁸R⁸⁸⁸, or —NR⁷⁷⁸R⁸⁸⁸ substituents, and wherein said ring optionally includes one or more heteroatoms other than the nitrogen to which R⁷⁸ and R⁸⁸ are attached;

R⁷⁷, R⁷⁸, R⁸⁷, R⁸⁸, R⁷⁷⁸, and R⁸⁸⁸ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl), or —N(C₀₋₄alkyl)(C₀₋₄alkyl) substituents;

or R⁷⁷, R⁷⁸, R⁸⁷, R⁸⁸, R⁷⁷⁸, and R⁸⁸⁸ are each independently aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl), or —N(C₀₋₄alkyl)(C₀₋₄alkyl) substituents;

n, m, j1, j1a, j2a, j4, j4a, j5a, j7, and j8 are each independently 0, 1, or 2; and aa and bb are each independently 0 or 1.

Additional, specific examples of IGF-1R kinase inhibitors include h7C10 (Centre de Recherche Pierre Fabre), an IGF-1 antagonist; EM-164 (ImmunoGen Inc.), an IGF-1R modulator; CP-751871 (figitumumab; Pfizer Inc.), an IGF-1 antagonist; lanreotide (Ipsen), an IGF-1 antagonist; IGF-1R oligonucleotides (Lynx Therapeutics Inc.); IGF-1 oligonucleotides (National Cancer Institute); IGF-1R protein-tyrosine kinase inhibitors in development by Novartis (e.g. NVP-AEW541, Garcia-Echeverria, C. et al. (2004) Cancer Cell 5:231-239; or NVP-ADW742, Mitsiades, C. S. et al. (2004) Cancer Cell 5:221-230); IGF-1R protein-tyrosine kinase inhibitors (Ontogen Corp); AG-1024 (Camirand, A. et al. (2005) Breast Cancer Research 7:R570-R579 (DOI 10.1186/bcr1028); Camirand, A. and Pollak, M. (2004) Brit. J. Cancer 90:1825-1829; Pfizer Inc.), an IGF-1 antagonist; the tyrphostins AG-538 and I-OMe-AG 538; BMS-536924, a small molecule inhibitor of IGF-1R; PNU-145156E (Pharmacia & Upjohn SpA), an IGF-1 antagonist; BMS 536924, a dual IGF-1R and IR kinase inhibitor (Bristol-Myers Squibb); AEW541 (Novartis); GSK621659A (Glaxo Smith-Kline); INSM-18 (Insmed); and XL-228 (Exelixis).

Antibody-based IGF-1R kinase inhibitors include any anti-IGF-1R antibody or antibody fragment that can partially or completely block IGF-1R activation by its natural ligand. Antibody-based IGF-1R kinase inhibitors also include any anti-IGF-1 antibody or antibody fragment that can partially or completely block IGF-1R activation. Non-limiting examples of antibody-based IGF-1R kinase inhibitors include those described in Larsson, O. et al (2005) Brit. J. Cancer 92:2097-2101 and Ibrahim, Y. H. and Yee, D. (2005) Clin. Cancer Res. 11:944s-950s, or being developed by Imclone (e.g. A12) or Schering-Plough Research Institute (e.g. 19D12; or as described in US Patent Application Publication Nos. US 2005/0136063 A1 and US 2004/0018191 A1). The IGF-1R kinase inhibitor can be a monoclonal antibody, or an antibody or antibody fragment having the binding specificity thereof.

Additional antibody-based IGF-1R kinase inhibitors can be raised according to known methods by administering the appropriate antigen or epitope to a host animal selected, e.g., from pigs, cows, horses, rabbits, goats, sheep, and mice, among others. Various adjuvants known in the art can be used to enhance antibody production.

Although antibodies useful in practicing the invention can be polyclonal, monoclonal antibodies are preferred. Monoclonal antibodies against IGF-1R can be prepared and isolated using any technique that provides for the production of antibody molecules by continuous cell lines in culture. Techniques for production and isolation include but are not limited to the hybridoma technique originally described by Kohler and Milstein (Nature, 1975, 256: 495-497); the human B-cell hybridoma technique (Kosbor et al., 1983, Immunology Today 4:72; Cote et al., 1983, Proc. Nati. Acad. Sci. USA 80: 2026-2030); and the EBV-hybridoma technique (Cole et al, 1985, Monoclonal Antibodies and Cancer Therapy, Alan R. Liss, Inc., pp. 77-96).

Alternatively, techniques described for the production of single chain antibodies (see, e.g., U.S. Pat. No. 4,946,778) can be adapted to produce anti-IGF-1R single chain antibodies. Antibody-based IGF-1R kinase inhibitors useful in practicing the present invention also include anti-IGF-1R antibody fragments including but not limited to F(ab′).sub.2 fragments, which can be generated by pepsin digestion of an intact antibody molecule, and Fab fragments, which can be generated by reducing the disulfide bridges of the F(ab′).sub.2 fragments. Alternatively, Fab and/or scFv expression libraries can be constructed (see, e.g., Huse et al., 1989, Science 246: 1275-1281) to allow rapid identification of fragments having the desired specificity to IGF-1R.

Techniques for the production and isolation of monoclonal antibodies and antibody fragments are well-known in the art, and are described in Harlow and Lane, 1988, Antibodies: A Laboratory Manual, Cold Spring Harbor Laboratory, and in J. W. Goding, 1986, Monoclonal Antibodies: Principles and Practice, Academic Press, London. Humanized anti-IGF-1R antibodies and antibody fragments can also be prepared according to known techniques such as those described in Vaughn, T. J. et al., 1998, Nature Biotech. 16:535-539 and references cited therein, and such antibodies or fragments thereof are also useful in practicing the present invention.

IGF-1R kinase inhibitors can alternatively be based on antisense oligonucleotide constructs. Anti-sense oligonucleotides, including anti-sense RNA molecules and anti-sense DNA molecules, would act to directly block the translation of IGF-1R mRNA by binding thereto and thus preventing protein translation or increasing mRNA degradation, thus decreasing the level of IGF-1R kinase protein, and thus activity, in a cell. For example, antisense oligonucleotides of at least about 15 bases and complementary to unique regions of the mRNA transcript sequence encoding IGF-1R can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as IGF-1R kinase inhibitors. IGF-1R gene expression can be reduced by contacting the tumor, subject or cell with a small double stranded RNA (dsRNA), or a vector or construct causing the production of a small double stranded RNA, such that expression of IGF-1R is specifically inhibited (i.e. RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494-498; Hannon, G. J. (2002) Nature 418:244-251; McManus, M. T. and Sharp, P. A. (2002) Nature Reviews Genetics 3:737-747; Bremmelkamp, T. R. et al. (2002) Science 296:550-553; U.S. Pat. Nos. 6,573,099 and 6,506,559; and International Patent Publication Nos. WO 01/36646, WO 99/32619, and WO 01/68836).

Ribozymes can also function as IGF-1R kinase inhibitors. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of IGF-1R mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as IGF-1R kinase inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

In the context of the methods of treatment of this invention, IGF-1R kinase inhibitors that inhibit both IGF-1R and IR kinases are used as a composition comprised of a pharmaceutically acceptable carrier and a non-toxic therapeutically effective amount of an IGF-1R kinase inhibitor that inhibits both IGF-1R and IR kinases compound (including pharmaceutically acceptable salts thereof).

The term “pharmaceutically acceptable salts” refers to salts prepared from pharmaceutically acceptable non-toxic bases or acids. When a compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

When a compound used in the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

Pharmaceutical compositions used in the present invention comprising an IGF-1R kinase inhibitor that inhibits both IGF-1R and IR kinases (including pharmaceutically acceptable salts thereof) as active ingredient, can include a pharmaceutically acceptable carrier and optionally other therapeutic ingredients or adjuvants. Other therapeutic agents may include those cytotoxic, chemotherapeutic or anti-cancer agents, or agents which enhance the effects of such agents, as listed above. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the IGF-1R kinase inhibitors that inhibit both IGF-1R and IR kinases (including pharmaceutically acceptable salts thereof) of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, an IGF-1R kinase inhibitor that inhibits both IGF-1R and IR kinases (including pharmaceutically acceptable salts of each component thereof) may also be administered by controlled release means and/or delivery devices. The combination compositions may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

An IGF-1R kinase inhibitor that inhibits both IGF-1R and IR kinases (including pharmaceutically acceptable salts thereof) used in this invention, can also be included in pharmaceutical compositions in combination with one or more other therapeutically active compounds. Other therapeutically active compounds may include those cytotoxic, chemotherapeutic or anti-cancer agents, or agents which enhance the effects of such agents, as listed above.

Thus in one embodiment of this invention, the pharmaceutical composition can comprise an IGF-1R kinase inhibitor that inhibits both IGF-1R and IR kinases in combination with an anticancer agent, wherein said anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition used fot this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably contains from about 0.05 mg to about 5 g of the active ingredient.

For example, a formulation intended for the oral administration to humans may contain from about 0.5 mg to about 5 g of active agent, compounded with an appropriate and convenient amount of carrier material that may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions used in the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions used in the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions for the present invention can be in a form suitable for topical sue such as, for example, an aerosol, cream, ointment, lotion, dusting powder, or the like. Further, the compositions can be in a form suitable for use in transdermal devices. These formulations may be prepared, utilizing an IGF-1R kinase inhibitor that inhibits both IGF-1R and IR kinases (including pharmaceutically acceptable salts thereof), via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions for this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing an IGF-1R kinase inhibitor that inhibits both IGF-1R and IR kinases (including pharmaceutically acceptable salts thereof) may also be prepared in powder or liquid concentrate form.

Dosage levels for the compounds used for practicing this invention will be approximately as described herein, or as described in the art for these compounds. It is understood, however, that the specific dose level for any particular patient will depend upon a variety of factors including the age, body weight, general health, sex, diet, time of administration, route of administration, rate of excretion, drug combination and the severity of the particular disease undergoing therapy.

The present invention further provides for any of the “methods of treatment” described herein, a corresponding “method for manufacturing a medicament” for use with the same indications and under identical conditions or modalities described for the method of treatment, characterized in that an IGF-1R kinase inhibitor that inhibits both IGF-1R and IR kinases is used, such that where any additional agents, inhibitors or conditions are specified in alternative embodiments of the method of treatment they are also included in the corresponding alternative embodiment for the method for manufacturing a medicament. The present invention also provides an IGF-1R kinase inhibitor that inhibits both IGF-1R and IR kinases for use in any of the methods of treatment for cancer described herein.

Many alternative experimental methods known in the art may be successfully substituted for those specifically described herein in the practice of this invention, as for example described in many of the excellent manuals and textbooks available in the areas of technology relevant to this invention (e.g. Using Antibodies, A Laboratory Manual, edited by Harlow, E. and Lane, D., 1999, Cold Spring Harbor Laboratory Press, (e.g. ISBN 0-87969-544-7); Roe B. A. et. al. 1996, DNA Isolation and Sequencing (Essential Techniques Series), John Wiley & Sons.(e.g. ISBN 0-471-97324-0); Methods in Enzymology: Chimeric Genes and Proteins”, 2000, ed. J. Abelson, M. Simon, S. Emr, J. Thorner. Academic Press; Molecular Cloning: a Laboratory Manual, 2001, 3r^{d} Edition, by Joseph Sambrook and Peter MacCallum, (the former Maniatis Cloning manual) (e.g. ISBN 0-87969-577-3); Current Protocols in Molecular Biology, Ed. Fred M. Ausubel, et. al. John Wiley & Sons (e.g. ISBN 0-471-50338-X); Current Protocols in Protein Science, Ed. John E. Coligan, John Wiley & Sons (e.g. ISBN 0-471-11184-8); and Methods in Enzymology: Guide to protein Purification, 1990, Vol. 182, Ed. Deutscher, M. P., Acedemic Press, Inc. (e.g. ISBN 0-12-213585-7)), or as described in the many university and commercial websites devoted to describing experimental methods in molecular biology.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

Experimental Details:

Introduction

The role of insulin-like growth factor receptor (IGF-1R) in tumor cell proliferation and survival is well established (1-3). IGF-1R is a receptor tyrosine kinase (RTK) with a di-dimeric structure, coordinated by disulfide bonding, and is activated upon binding the growth factor ligands IGF-1 and IGF-2 (4). IGF-1R couples to the PI3K-AKT signaling pathway, via interactions with the adaptor protein insulin receptor substrate (IRS). IGF-1R is required for oncogenic transformation and tumorigenesis (5, 6), and disruption of IGF-1R activity by either genetic (7, 8) or pharmacological (9-15) approaches can reduce tumor cell proliferation and promote apoptosis. Increased expression of IGF-1R and its ligands is associated with etiology, progression, and prognosis for many human cancer types (16, 17). IGF-1R signaling is a key contributor of resistance to cytotoxic chemotherapeutics, ionizing radiation, and certain targeted agents, including inhibitors of EGFR, HER2, and mTOR (16-22). IGF-1R has been intensely pursued as a cancer target, and both biologic and small molecule tyrosine kinase domain inhibitors (TKIs) of IGF-1R are under investigation in oncology clinical trials (23-26). Given the important role for IGF-1R signaling as an adaptive survival mechanism against a diverse array of anti-tumor agents, combination therapies centered on IGF-1R inhibitors are being widely explored.

IGF-1R is closely related to the insulin receptor (IR); sharing 70% amino acid identity overall and 84% identity within the catalytic (tyrosine kinase) domains (27, 28). IGF-1R and IR can homo- or hetero-dimerize, and dimers are differentially activated by the ligands, insulin, IGF-1 and IGF-2. Insulin is the classical ligand for IR and most potently activates IR homodimers, however the ability of IGF-2 to activate IR is also well established (29-31). In addition to IR's role in metabolic signaling for tissues that regulate glucose homeostasis, IR can also promote cell proliferation and survival. Increased IGF-2-mediated IR signaling can rescue mouse embryonic development to prevent dwarfism in mice caused by knockout of the IGF1R gene (30). A growing body of data indicates that tumor cells can also exploit IR to promote proliferation and survival (31-33). Ectopic expression of IR oncogenically transforms NIH3T3 fibroblasts and 184B5 mammary epithelial cells (34, 35). Ablation of pancreatic islet cells in mice reduces the growth rates of implanted xenograft tumors suggesting that insulin-mediated IR signaling in tumor cells can promote tumor growth (44-46). Epidemiological studies have shown that elevated levels of insulin and C-peptide are associated with poor prognosis and accelerated tumor growth for a number of tumor types including carcinomas of the breast, prostate, colon, endometrium, liver and ovary (1, 36, 37). Furthermore, clinical studies of an inhaled form of insulin for the treatment of Type I diabetes were recently halted due to an increased risk of developing lung cancer (38).

Compensatory RTK signaling is emerging as a major mode of resistance to anti-tumor agents that selectively target a single RTK in tumor cells. Resistance to inhibition of EGFR or HER2 can be mediated by an adaptive increase in MET or IGF-1R activity (39, 40). There are also data showing reciprocal crosstalk between IGF-1R and IR. In mouse embryogenesis, compensatory IR signaling, driven by IGF-2 can fully maintain normal embryonic growth in IGF-1R^{-/-} mice, while double knockouts, IGF-1R^{-/-} IR^{-/-}, are non-viable (30). In osteoblasts, where IGF-1R signaling stimulates growth and differentiation, genetic ablation of IGF1R results in increased IR activation that is associated with enhanced insulin-driven AKT and ERK signaling (41). Upon loss of IGF-1R function, osteoblasts shift from IGF- to insulin-mediated growth and differentiation. In a reciprocal manner, knockout of IR in keratinocytes is associated with a compensatory increase in IGF-driven IGF-1R signaling (42). Therefore, upregulated IR signaling can compensate for loss of IGF-1R, and vice versa, to maintain cellular function in a number of biological systems. More recent data have indicated that crosstalk between IR and IGF-1R may also occur in tumor cells as increased insulin signaling is observed upon downregulation of IGF-1R (43).

Although mitogenic signaling by IR has been demonstrated in some tumor cell models, co-dependence on IGF-1R and IR has not been extensively studied. We sought to determine whether IR can drive tumor cell survival signaling and mediate resistance to selective inhibition of IGF-1R and if co-inhibition of IR and IGF-1R could provide superior inhibition of AKT signaling as well as inhibition of tumor cell proliferation and survival compared to selective inhibition of IGF-1R. IGF-1R and IR are co-expressed in a wide range of human tumor cell lines, and treatment with a neutralizing monoclonal antibody (MAb) specifically directed against IGF-1R (MAB391) resulted in increased phosphorylation of IR. Furthermore, treatment of tumor cells and xenografts with the anti-IGF-1R MAb alone resulted in only partial reduction of phospho-IRS1 and phospho-AKT, whereas OSI-906, a selective dual inhibitor of IGF-1R and IR, more effectively reduced phospho-IRS1 and phospho-AKT in several human tumor cell lines. In xenograft tumors with readily detectable basal levels of phospho-IGF-1R and phospho-IR, dual receptor inhibition by OSI-906 resulted in enhanced anti-tumor activity compared to a selective anti-IGF-1R MAb. Either insulin or IGF-2 was able to activate the IR-AKT pathway and decrease the sensitivity of tumor cells to selective inhibition of IGF-1R by the anti-IGF-1R MAb. In contrast, activation of the IR-AKT pathway by insulin or IGF-2 was fully blocked by OSI-906. Collectively, these data support the hypothesis that drugs co-targeting IGF-1R and IR, such as OSI-906, may provide superior efficacy compared to MAbs selective for IGF-1R by preventing IR:IGF-1R mediated compensatory signaling.

Materials and Methods

IGF-1R/IR inhibitors: OSI-906 was synthesized as previously described (13) and dissolved in DMSO at 10 mmol/L for use in in vitro cellular assays. MAB391, IGFBP3, and the IGF-2 neutralizing antibody were from R&D Systems (Minneapolis, Minn.).

Cell Lines and Culture. Human cancer cell lines, were obtained from American Type Culture Collection (ATCC, Manassas, Va.), or the following additional indicated sources, and cultured in media as described. Tumor types are also indicated: H295R (adrenocortical carcinoma; ATCC), NCI-H322 (NSCLC; ECACC), NCI-H460 (NSCLC; ATCC), SW1573 (NSCLC; ATCC), H1703 (NSCLC; ATCC), BxPC3 (pancreatic; ATCC), OVCAR5 (ovarian; NCI;), MDAH-2774 (ovarian; ATCC), Igrov1 (ovarian; NCI), GEO (colon; Roswell Park Cancer Institute (RPCC)), HT-29 (colon; ATCC), RKO (colon; ATCC), H226 (NSCLC; ATCC), 8226 (myeloma; ATCC), H929 (myeloma; ATCC), U266 (myeloma; ATCC), SKES1 (Ewings sarcoma; ATCC), RDES (Ewings sarcoma; ATCC), RD (rhabdomyosarcoma; ATCC), DU4475 (breast; ATCC), SKNAS (neuroblastoma; ATCC), 2650 (nasal SCC; ATCC), OVCAR4 (ovarian; NCI), A673 (Ewings sarcoma; ATCC), BT474 (breast; ATCC), 1386 (oral SCC; MSKCC, NY), 1186 (SCCHN; MSKCC, NY), Colo205 (colon; ATCC), HCT-15 (colon; ATCC), Fadu (oral SCC; ATCC), SKBR3 (breast; ATCC), 1483 (HNSCC; MSKCC, NY), HSC-2 (HNSCC; RIKEN BioResource Center, Tsukuba, Ibaraki, 305-0074, Japan). Cells were maintained at 37° C. in an incubator under an atmosphere containing 5% CO₂. The cells were routinely screened for the presence of mycoplasma (MycoAlert, Cambrex Bio Science, Baltimore, Md.). For growth inhibition assays, cells were plated and allowed to proliferate for 24 hours. After 24 hours, cells had reached approximately 15% confluency, at which time serial dilutions of OSI-906 were added and the cells grown for a further 72 hours. Cell viability was assayed using the Cell Titer-Glo reagent (Promega Corp., Madison, Wis.).

Preparation of Protein Lysates and Western Blotting: Lysates for Western blotting were prepared as previously described (44). Antibodies included: IGF-1R (Santa Cruz), IR (Santa Cruz), phospho-p42/p44 (Cell Signaling Technologies), phospho-Akt (S473) (Cell Signaling Technologies), phospho-Akt (T308) (Cell Signaling Technologies), phosho-S6 (235/236) (Cell Signaling Technologies), phosphor-PRAS40 (Cell Signaling Technologies), and phosphor-IRS-1-Y612 (Biosource). Where indicated, 40 ng/ml IGF1/2 ligands or insulin (5 μIU/ml or 50 μ IU/ml) were added for 5 minutes prior to lysis.

Analysis of RTK phosphorylation via a proteome array: Proteome Profiler arrays containing capture antibodies for 42 RTKs were from R&D systems (RTK Proteome Profile; R&D Systems, Minneapolis, Minn.) and processed according to manufacturer's protocol. pIGF-1R and pIR were determined by RTK capture array. Proteome profiler arrays housed 42 different RTKs. RTKs included on the array include: HER1, HER2, HER3, HER4, FGFR1, FGFR2a, FGFR3, FGFR4, IR, IGF-1R, Axl, Dtk, Mer, HGFR, MSPR, PDGFRα, PDGFRβ, SCFR, Flt-3, M-CSFR, c-Ret, ROR1, ROR2, Tie-1, Tie-2, TrkA, TrkB, TrkC, VEGFR1, VEGFR2, VEGFR3, MuSK, EphA1, EphA2, EphA3, EphA4, EphA6, EphA7, EphB1, EphB2, EphB4, EphB6. This array was used as an RTK capture assay for determining pIGF-1R and pIR levels. Capture antibodies specific to each RTK are used to bind RTKs in tumor cell extracts. An HRP-conjugated pan anti-phospho-tyrosine antibody is used to specifically detect phosphorylated RTKs.

Taqman Assays: The Gene Expression Assays for the genes IGF2, IGF1, IGF-1R, and IR were obtained from Applied Biosystems, Foster City, Calif. Quantitation of relative gene expression was conducted as described by the manufacturer using 50 ng of template. In order to determine relative expression across cell lines, amplification of IGF axis gene was compared to amplification of the gene for β-actin. All data were normalized to the 4^{th} quartile expression for a given gene within the 32 cell line panel. Gene expression assays were obtained from Applied Biosystems. Gene expression assays for IGF1 (Hs01547656), IGF2 (Hs01005963), IR (Hs00961557), and IGF-1R (Hs99999020) were inventoried. The gene expression assay for IRA was custom prepared for the sequences: INSRA probe (6FAM-CCC AGG CCA TCT CGG AAA CGC-TAMRA), INSRA forward primer (CTG CAC CAC AAC GTG GTT TTC GT), and INSRA reverse primer (ACG GCC ACC GTC ACA TTC). The IRA gene expression assays were previously described, K. Kalli et al. (2002) Endocrinology, 143(9), 3259-67. N. B. TAMRA is 6′carboxytetramethylrhodamine; 6-FAM is 6′carboxyfluorescein.

Animals: Female nu/nu CD-1 mice (6-8 weeks, 22-25 g) were purchased from Charles River Laboratories (Wilmington, Mass.) and maintained in an AAALAC-accredited facility at OSI Pharmaceuticals as described previously (12).

In vivo pharmacodynamic analysis: To assess the ability of OSI-906 or MAB391 to inhibit IGF-1R or IR phosphorylation in tumor tissue, female nu/nu CD-1 mice were implanted s.c. with tumor cells as described previously (12). Animals with established tumors of 300±50 mm³ size were dosed orally with OSI-906 dissolved in 25 mM tartaric acid or i.p. with MAB391 diluted in PBS at indicated doses. Tumor samples were collected at specified time points and snap frozen in liquid nitrogen. Tumor lysates were prepared by homogenizing samples in Precellys 24 homogenizer (MO Bio Laboratories, Inc., CA) with tumor lysis buffer (1% Triton X-100, 10% glycerol, 50 mM HEPES (ph 7.4), 150 mM NaCl, 1.5 mM MgCl2, 1 mM EDTA supplemented with fresh protease inhibitor cocktail (Sigma, Mo.), phosphatase inhibitor cocktail (Sigma, Mo.), 10 mM NaF and 1 mM sodium orthovanadate). Tissue homogenates were clarified by centrifugation at 14,000 g for 5 min at 4° C. and supernatants were then analyzed by Western blot or phospho-RTK array as indicated.

In vivo anti-tumor efficacy studies: Cells were harvested and implanted s.c. in the right flank of nu/nu CD-1 mice as described previously (12). Tumors were allowed to establish to 200±50 mm³ in size before randomization into various treatment groups. OSI-906 and MAB391 were administered as indicated. Tumor volumes were determined from caliper measurements using the formula V=(length×width²)/2. Tumor sizes and body weights were measured twice weekly. Tumor growth inhibition (TGI) was determined at different time points by the following formula: % TGI={1−[(Tₜ/T₀)/(Cₜ/C₀)]/1[C₀/Cₜ}×100, where Tₜ=median tumor volume of treated at time t, T₀=median tumor volume of treated at time 0, Cₜ=median tumor volume of control at time t, and C₀=median tumor volume of control at time 0. Mean TGI was calculated for the entire dosing period, with a mean TGI of 50% considered to be minimal response required for efficacy.

Results and Discussion

Tumor cells with elevated expression of genes associated with the IGF-1R/IR signaling axis are sensitive to OSI-906.

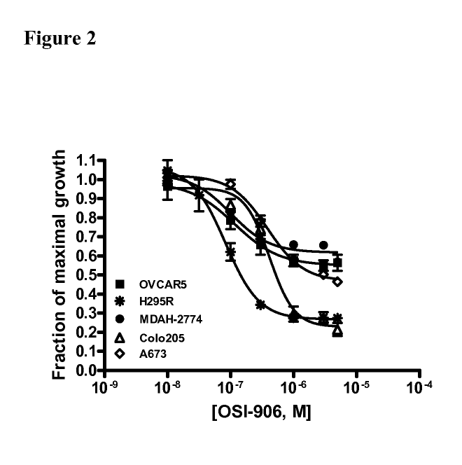

We sought to determine if gene expression or mutations within the IGF-1R/IR axis were predictive of sensitivity to OSI-906, a small molecule dual inhibitor of IGF-1R and IR. OSI-906 selectively inhibits both IGF-1R (IC₅₀=35 nM) and IR (IC₅₀=75 nM) and is far less potent (<50% inhibition at 1 μM) against a broad panel (n=116) of additional RTKs and other protein kinases (45). A panel of 32 tumor cell lines representing ten tumor types was selected based on differential sensitivity to OSI-906 in cell proliferation assays. Cell lines were categorized as either sensitive (EC₅₀<1 μM) or insensitive (EC₅₀>10 μM) to OSI-906 (FIG. 1A). For sensitive tumor cell lines, growth inhibition by OSI-906 was dose-dependent (FIG. 1B). Mutations in KRAS or BRAF are reported to decrease sensitivity to the anti-EGFR antibody cetuximab, however, it was found herein that these mutations occurred frequently in OSI-906-sensitive tumor cell lines. Greater than 50% of the OSI-906-sensitive tumor cells harbored mutations in either KRAS or BRAF, while these mutations were less frequent (<25%) in OSI-906-insensitive tumor cells, (FIG. 1A). In contrast, mutations in PIK3CA were observed in nearly half (6/13) of the OSI-906-insensitive tumor cell lines, but did not occur in any cell line that was sensitive to OSI-906. IGF-1R and IR couple very strongly to the PI3K-AKT pathway, and therefore mutations resulting in constitutive downstream signaling may mitigate the activity of IGF-1R/IR RTK inhibitors. Expression of IGF1, IGF2, IGF-1R and IR mRNAs, was measured by quantitative RT-PCR. For each gene, expression was normalized to the fourth quartile of expression for that gene within the 32-cell line panel. The cell lines were then ranked according to collective expression of ligands and receptors. Cell lines exhibiting the highest expression of genes in the IGF axis were sensitive to OSI-906 (FIG. 2). Among 19 OSI-906-sensitive cell lines, 14 exhibited expression of IGF1 or IGF2 mRNAs at levels that fell within the top quartile of expression across the entire 32-cell line panel. Interestingly, expression of IGF1 and IGF2 mRNAs were nearly mutually exclusive, with autocrine IGF1 mRNA expression frequent in tumor cells derived from hematologic malignancies (U266, H929, 822) or sarcomatoid tumor types (A673, RDES, SKES, RDES), and IGF2 mRNA expression frequent in tumor cells of epithelial derivation (GEO, HT29, MDAH-2774, DU4475, H322). In 9/19 of the OSI-906 sensitive cell lines, high (top quartile) co-expression of mRNAs encoding ligand (either IGF1 or IGF2) was observed along with mRNAs encoding receptor (either IGF-1R or IRA). In contrast, elevated co-expression of ligand and receptor mRNAs was not observed for any of the 13 OSI-906-insensitive cell lines evaluated. These data support a model in which elevated co-expression of receptor-ligand pairs in the IGF-1R/IR axis, consistent with tumor cell autocrine signaling, may be predictive for response to OSI-906.

Thus the sum of the measured expression levels for the transcripts expressed from the IGF-1, IGF-2, IGF-1R and IR genes (i.e. expression level index) is predictive of tumor cell sensitivity to IGF-1R kinase inhibitors such as OSI-906, with tumor cells having values for such an index equal to or greater than that for RDES or SK-N-AS tumor cells having high sensitivity, and thus patients with tumors comprising tumor cells with such index values are likely to be responsive to IGF-1R kinase inhibitors such as OSI-906.

In tumor xenograft studies, using tumor cells of a variety of tumor cell types that all have high sensitivity to OSI-906 in culture in vitro (<1 μM EC50), the tumors are also consistently inhibited in vivo with a high pencentage tumor growth inhibition (TGI) (e.g. For the following tumor cells, the indicated % TGI was obtained after treatment with OSI-906 in vivo for 10-14 days: H295R: 85%; SKNAS: 71%; BxPC3: 56%; Colo205: 90%). In contast, in similar studies, using tumor cells that have low sensitivity to OSI-906 in culture in vitro (>10 μM EC50), the tumors are inhibited in vivo with only a low pencentage tumor growth inhibition (TGI) (e.g. For the following tumor cells, the indicated % TGI was obtained after treatment with OSI-906 in vivo for 10-14 days: FaDu: <30%; H460: <30%). These data indicate that sensitivity to IGF-1R kinase inhibitors such as OSI-906 in tumor cell culture is predictive of tumor sensitivity in vivo.

Inhibition of IGF-1R is Associated with a Compensatory Increase in IR Signaling.

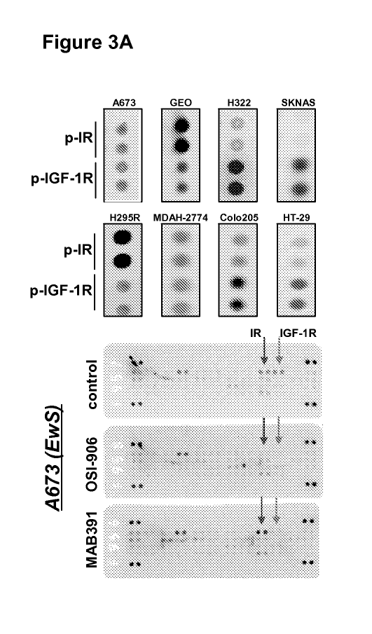

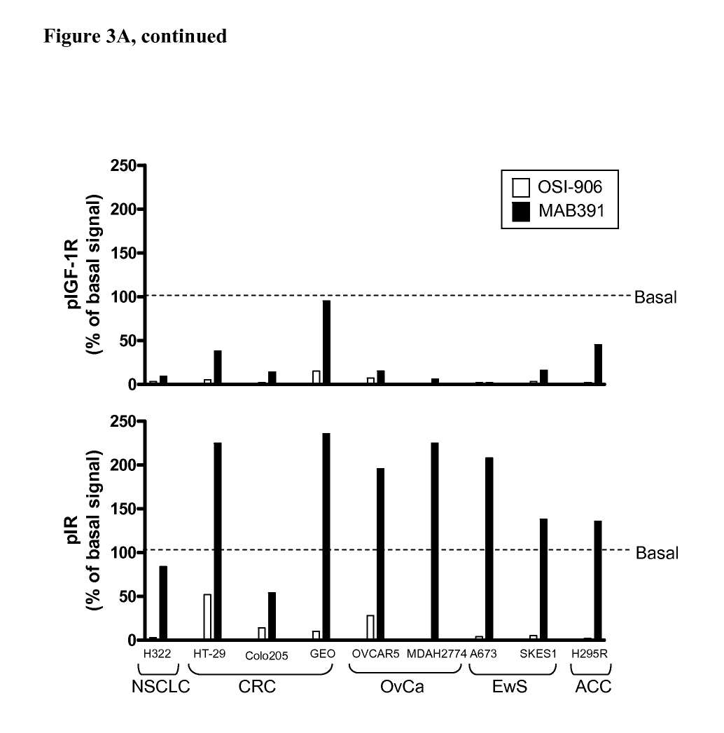

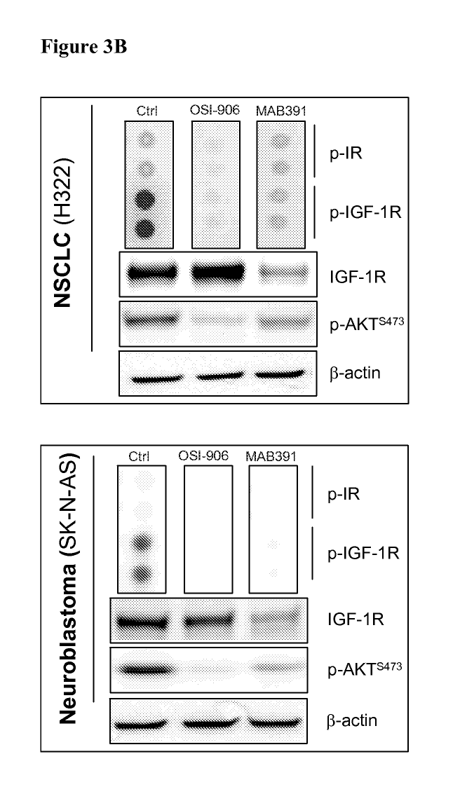

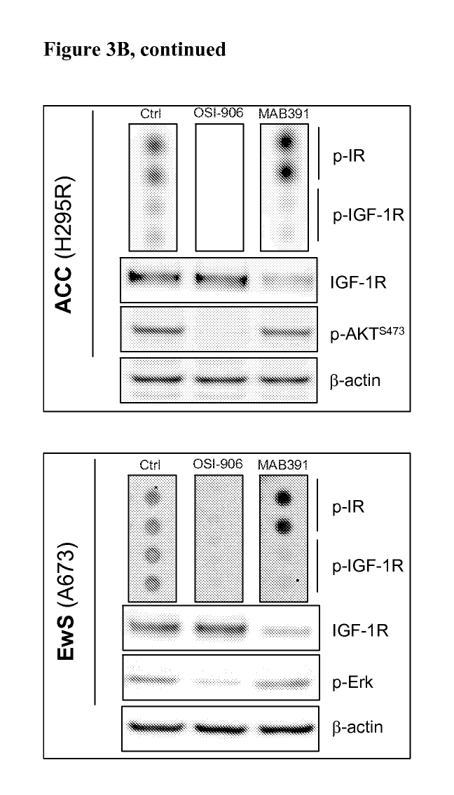

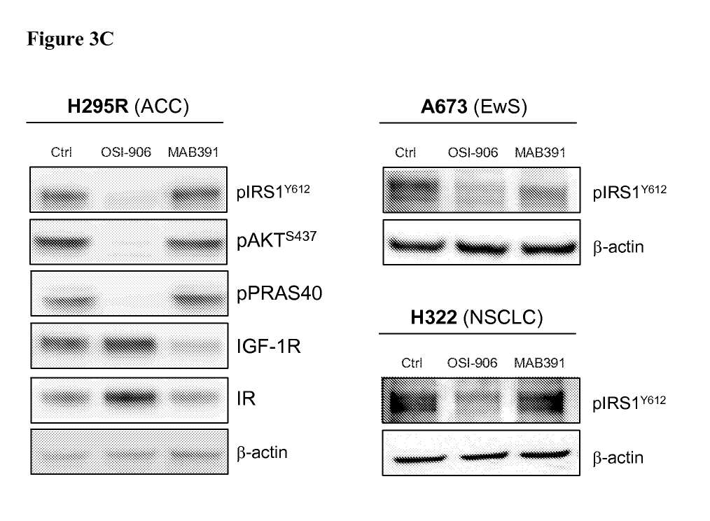

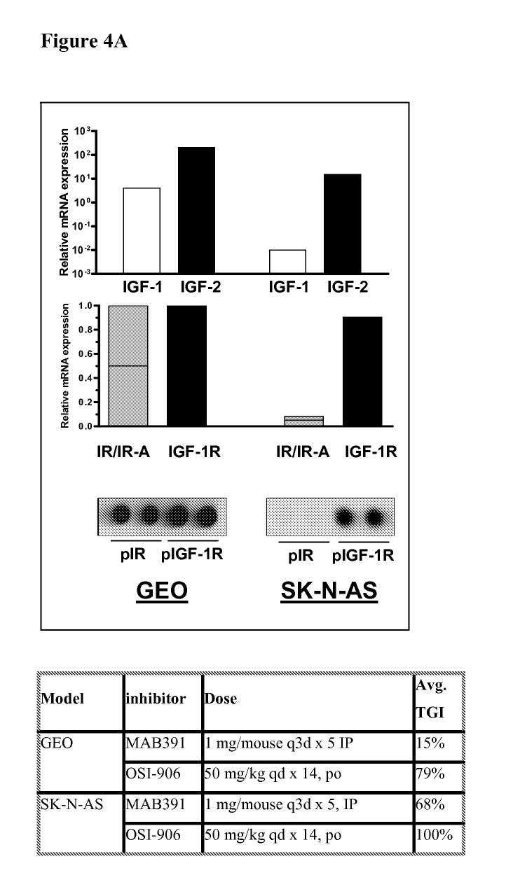

Phospho-IGF-1R and phospho-IR are often simultaneously detectable in human tumor cell lines (FIG. 3A, left panel). We sought to determine whether co-inhibition of IGF-1R and IR was required for maximal inhibition of downstream survival signaling through IRS1 and AKT in human tumor cell lines. OSI-906 was compared to the selective anti-IGF-1R MAb, MAB391, in tumor cell signaling assays measuring the phosphorylation of IGF-1R and IR as well as cytoplasmic signaling intermediates, including phospho-IRS1^{Y612}, phospho-AKT, and phospho-ERK. MAB391 is believed to exhibit pharmacological properties similar to many anti-IGF-1R MAb drug candidates currently in clinical development by inhibiting signaling from both IGF-1R homodimers and IGF-1R/IR heterodimers but not from IR/IR homodimers. The effects of OSI-906 (3 μM) or MAB391 (3 μg/mL) on the phosphorylation of IGF-1R and IR was determined across a panel of nine tumor cell lines representing several tumor types. OSI-906 decreased phospho-IGF-1R by >90% and phospho-IR by >50% in each cell line tested (FIG. 3A). MAB391 was similarly effective at decreasing phospho-IGF-1R, but only moderately inhibited (50%) phospho-IR in one of the nine tumor cell lines tested, Colo205. Interestingly, MAB391 treatment resulted in a substantial increase in detectable phospho-IR in 7/9 cell lines evaluated, supporting a model of compensatory IGF-1R/IR signaling.

The ability of IGF-1R inhibitors to block downstream AKT and ERK signaling is associated with their ability to decrease tumor cell proliferation and survival. In SK-N-AS (neuroblastoma) tumor cells, phospho-IGF-1R but not phospho-IR was detectable and associated with ability of either OSI-906 or MAB391 to decrease phospho-AKT levels (FIG. 3B). However, in 3/4 cell lines with detectable basal phospho-IR and phospho-IGF-1R (H322, H295R, and A673), OSI-906 decreased phospho-AKT or phospho-ERK levels to a greater extent than did MAB391. This was especially striking in the H295R ACC cell line, which showed the highest phospho-IR/phospho-IGF-1R ratio. Despite its ability to promote a 70-90% decrease in IGF-1R expression (presumably by internalization and degradation), MAB391 was still unable to maximally decrease phospho-AKT. These data support a role for IR in maintaining downstream signaling when IGF-1R is selectively inhibited.

IRS1 is a substrate of IGF-1R/IR and serves as a signaling intermediary for the PI3K-AKT pathway. Inhibition of pIRS1^{Y612} is associated with activity of IGF-1R inhibitors (47). In A673, H322, and H295R tumor cell lines OSI-906, but not MAB391, strongly inhibited phosphorylation of IRS1^{Y612} (FIG. 3C). In H295R cells, inhibition of the phosphorylation of IRS1 and AKT by OSI-906, but not MAB391, was associated decreased phospho-PRAS40, a direct substrate of AKT. These data indicate that the IR can contribute to activation of downstream AKT signal in tumor cells at the level of IRS1.

Data indicate that IR may also play a pro-survival role upon treatment of tumor cells with a chemotherapeutic agent. We find that following treatment of A673 tumor cells with doxorubicin (300 nM) there is upregulated phosphorylation of both IR and IGF-1R, and this correlates with increased phosphorylation of downstream signaling at the level of pERK. OSI-906 inhibits both IR and IGF-1R, while MAB391 does not fully inhibit pIR. This results in greater inhibition of pERK by OSI-906, compared with MAB391.

Dual Inhibition of IR and IGF-1R is Associated with Enhanced Anti-Tumor Activity In Vivo.

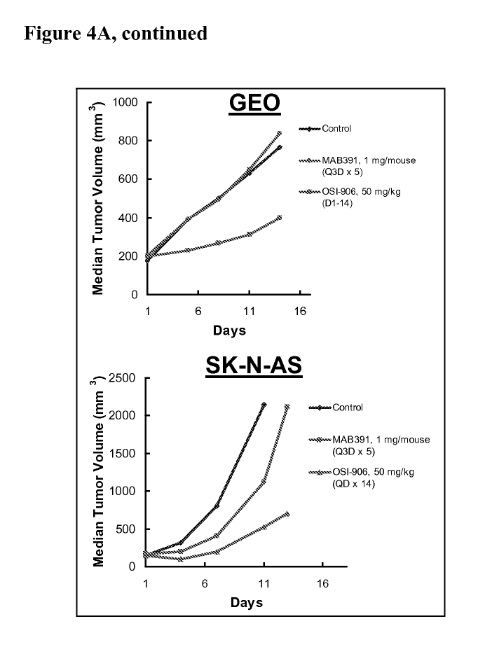

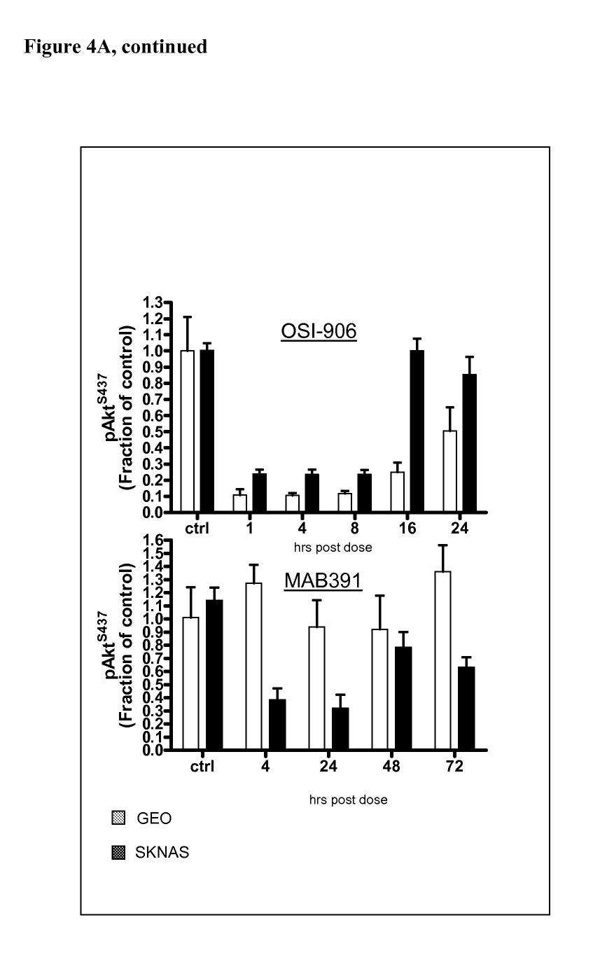

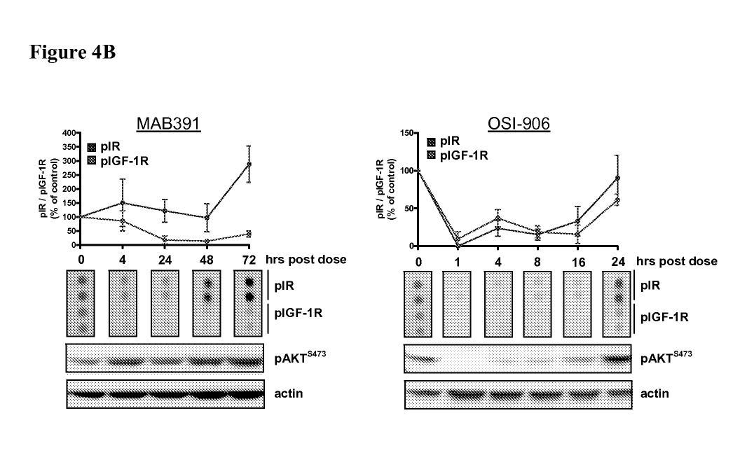

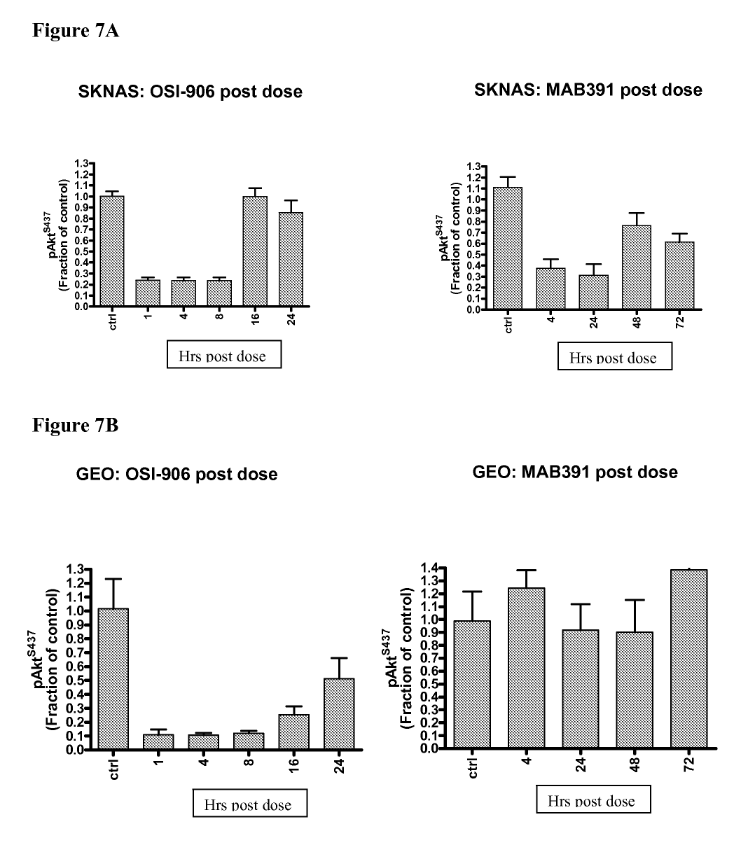

Dual inhibition of IR and IGF-1R was investigated in vivo in two xenograft tumor models, GEO (CRC) and SK-N-AS (neuroblastoma). Both GEO and SK-N-AS tumor cells express IGF2 mRNA Both cell lines express similar levels of IGF1R mRNA, however GEO cells, but not SK-N-AS cells, also express IR mRNA (FIG. 4A). SK-N-AS tumor cells have readily detectable levels of basal phospho-IGF-1R, but not phospho-IR, whereas GEO cells contain high levels of both phospho-IGF-1R and phospho-IR (FIGS. 3B and 4A). In the SK-N-AS tumor model, OSI-906, administered at 50 mg/kg once-daily for 14 days, resulted in significant mean tumor growth inhibition (TGI) of 100% over the dosing period. MAB391 administered at 1 mg every three days intraperitoneally was also efficacious (68% mean TGI) in this model. Treatment with a single dose of either OSI-906 or MAB391 resulted in decreased phospho-AKT (>60% compared to vehicle control) with partial recovery at later timepoints (FIG. 4A and FIG. 7). Similar effects on phospho-PRAS40, a substrate of AKT, were also observed (data not shown). In the GEO xenograft model, treatment with OSI-906 at 50 mg/kg once-daily for 14 days resulted in significant inhibition of tumor growth (mean TGI of 79% over the dosing period), while MAB391, administered every three days for a total of 5 doses was completely inactive in this model (FIG. 4A). Both drugs were well tolerated, with minimal (<10%) body weight loss. The efficacy of OSI-906 in the GEO model was reflected by decreased phospho-AKT in tumors, whereas treatment with MAB391 did not result in decreased phospho-AKT (FIG. 4A, and data not shown). Differential effects of OSI-906 and MAB391 on phospho-AKT correlated with their effects on phospho-IR (FIG. 4B). Although treatment with either OSI-906 or MAB391 resulted in decreased phospho-IGF-1R (>50% inhibition throughout the dosing period), only treatment with OSI-906 resulted in a significant decrease in phospho-IR (>50% for at least 16 hours). In contrast, treatment with MAB391 had no significant effect on phospho-IR for the first 48 hours after dosing, and by 72 hours after dosing, phospho-IR levels increased by greater than two-fold compared to vehicle treated control tumors (FIG. 4B). These data are consistent with our in vitro observations, where treatment with MAB391 resulted in a compensatory increase in phospho-IR. Therefore, in GEO tumors co-targeting of IGF-1R and IR resulted in enhanced inhibition of phospho-AKT, corresponding with improved tumor growth inhibition. Taken together, the pharmacodynamic and efficacy studies in the GEO and SK-N-AS tumor models indicate that inhibition of both IGF-1R and IR may be required for optimal efficacy in cancers where both receptors are present and activated. The data also indicate that tumor cells with insulin receptor levels (e.g. IR transcript levels (i.e. IR-A and/or IR-B)) equal to or greater than GEO tumor cells will be insensitive to inhibition by an anti-IGF-1R antibody, and thus patients with tumors comprising tumor cells with such levels are likely to be unresponsive to anti-IGF-1R antibody therapy.

The data also indicates that certain tumor cells with high phospho-IR/phospho-IGF-1R ratio (e.g. A673 cells, FIG. 3B), indicative of a high level of active insulin receptor, will be insensitive to anti-IGF-1R antibodies. This data indicates that tumor cells with insulin receptor levels (e.g. IR transcript levels (i.e. IR-A and/or IR-B)) equal to or greater than A673 tumor cells will be insensitive to inhibition by an anti-IGF-1R antibody, and thus patients with tumors comprising tumor cells with such levels are likely to be unresponsive to anti-IGF-1R antibody therapy.

OSI-906 Inhibits Insulin-Driven AKT Signaling.

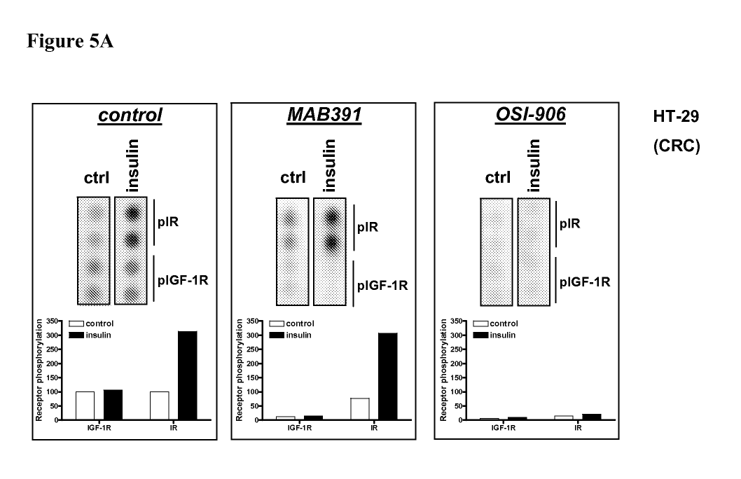

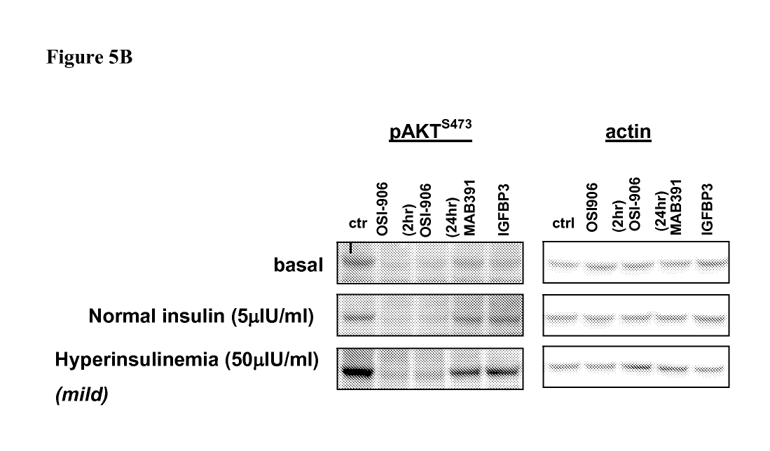

Elevated insulin is associated with poor prognosis in a number of tumor types (1, 36, 37). It was confirmed that insulin at 50 μIU/mL, a level corresponding to mild fasting hyperinsulinemia in humans, increased both phospho-IR and phospho-AKT, but not phospho-IGF-1R, in HT-29 CRC cells (FIGS. 5A and B). Only OSI-906 fully inhibited phospho-IGF-1R, phospho-IR and phospho-AKT in HT-29 cells treated with either 5 or 50 μ IU/mL insulin, corresponding to normal fasting insulin levels and mild hyperinsulemic levels, respectively. In contrast, MAB391 only significantly reduced phospho-IGF-1R content in HT-29 and had minimal to no effects on phoshpo-IR and phospho-AKT under all conditions tested (FIGS. 5A and B). Treatment with IGFBP3, which can neutralize IGF-1 or IGF-2 ligands, but not insulin, resulted in effects on phospho-AKT similar to those oberseved for MAB391 and far less significant than those caused by OSI-906 (FIG. 5B). These data indicate that even mild increases in insulin levels may provide survival signals to tumor cells which may mitigate the activity of IGF-1R-selective therapies.

IGF-2 can Drive IR-AKT Signaling.

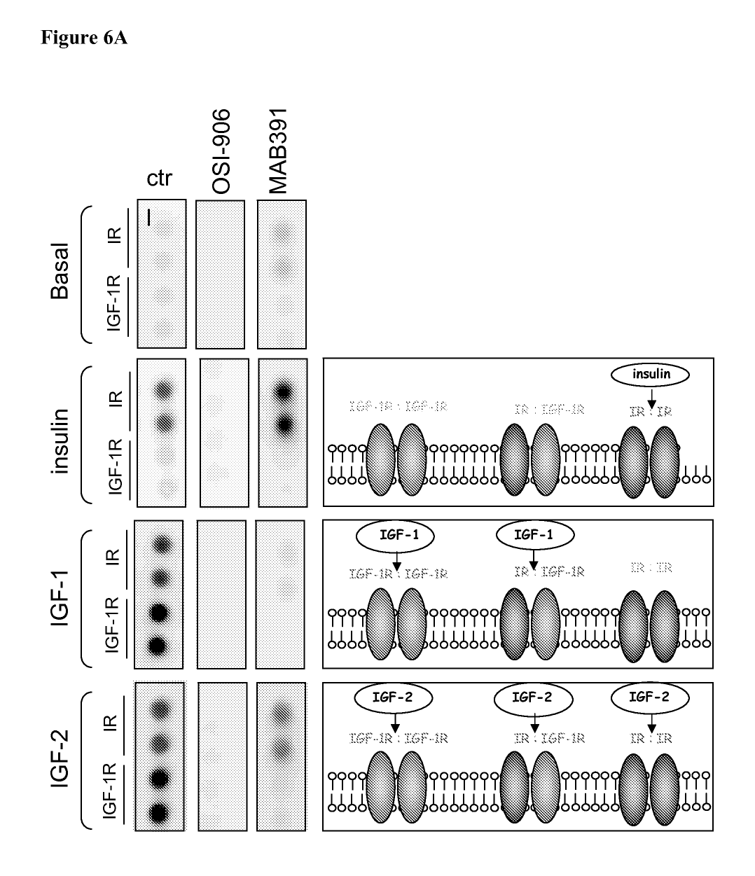

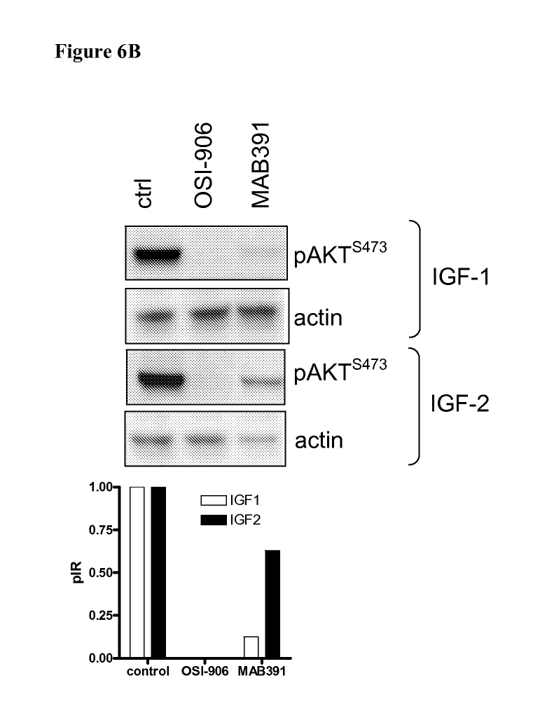

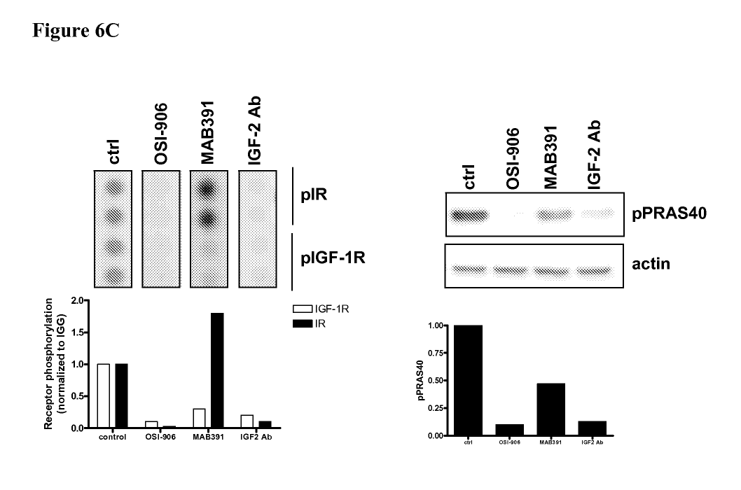

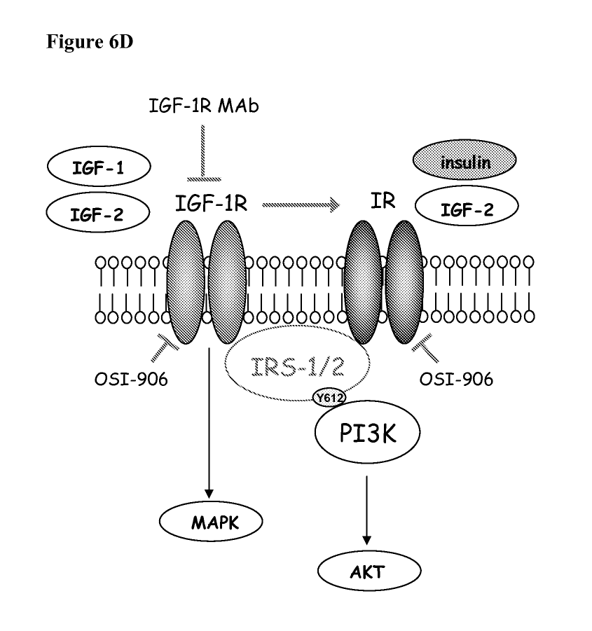

Increased expression of IGF-2 has been observed in a number of tumor types, caused in some instances by loss of imprinting (LOI) at the IGF2 locus ⁽⁴⁸⁻⁵³⁾. LOI for IGF2 occurs in subsets of a number of human cancers including colorectal carcinomas (CRC) and adrenocortical carcinomas (ACC). LOI for IGF2 and increased IGF2 mRNA expression are observed in greater than 90% of ACC tumors (54). Since IGF-2 can activate IR, we asked whether it also signals through AKT in an autocrine loop independently of IGF-1R. MDAH-2774 OvCa tumor cells use an IGF-2 autocrine loop, and are sensitive to OSI-906 in vitro. MDAH-2774 cells were treated with OSI-906 or MAB391 alone, or in the presence of insulin, IGF-1, or IGF-2. Insulin (50 μIU/mL) activated IR, but not IGF-1R, as reflected by increased receptor phosphorylation (FIG. 6A). Treatment with 40 ng/mL IGF-1 or IGF-2 increased IR and IGF-1R phosphorylation. IGF-1 presumably increased phospho-IR within the context of IGF-1R/IR heterodimers, while IGF-2 presumably increased phospho-IR within the context of either IGF-1R/IR heterodimers or IR/IR homodimers. OSI-906 fully inhibited IGF-1R and IR phosphorylation in all cases. While MAB391 also inhibited phospho-IGF-1R under all conditions, it had varied effects on phospho-IR, which were dependent on the stimulating ligand. Under basal conditions, MAB391 activated phospho-IR by approximately two-fold. 50 μIU/ml insulin promoted a 7-fold increase in phospho-IR, and this was potentiated to greater than 12-fold when cells were co-treated with MAB391. Both IGF-1 and IGF-2 promoted increased phospho-IR, however, while MAB391 completely inhibited phospho-IR driven by IGF-1, it did not fully inhibit phospho-IR driven by IGF-2. Both ligands promoted downstream AKT signaling. MAB391 fully inhibited IGF-1 stimulation of phospho-AKT (FIG. 6B). However, in cells pretreated with MAB391, IGF-2 could partially rescue AKT phosphorylation. These data indicate that the potential for differential efficacy for agents which specifically inhibit IGF-1R, compared to those that co-inhibit IGF-1R and IR, may be affected by the levels of various ligands available within the intratumoral compartment. High intratumoral levels of IGF-2 and/or insulin may indicate that co-targeting of IGF-1R and IR is required for maximal efficacy, since both of these ligands can activate IR homodimers.

To further validate IGF-2-driven IR-AKT signaling, the ability of an IGF-2 neutralizing antibody to decrease phospho-IR and phospho-AKT was evaluated. Under basal culture conditions, MAB391 activated IR in a compensatory manner. However, neutralization of IGF-2 achieved near complete inhibition of the phosphorylation states for both IGF-1R and IR (FIG. 6C). Furthermore, greater inhibition phospho-PRAS40 was caused by the IGF-2 neutralizing antibody, compared to MAB391. These data indicate that the enhanced activity for OSI-906 against the IR-AKT pathway is specific, and indicate that IGF-2, in addition to insulin can activate IR signaling in tumor cells in order to maintain survival signaling.

Discussion/Conclusions

The observation that a range of RTKs can function to drive tumorigenesis has revolutionized drug discovery and development efforts in recent decades. However, tumor cells exhibit a high degree of signaling plasticity, which can contribute to adaptive survival in the presence of RTK inhibitors, and identifying the mechanisms of acquired resistance for these agents is a major goal toward optimizing their design and individualizing their use in the clinic. Multiple RTKs can be activated simultaneously within a single cell, and crosstalk can exist between them. Crosstalk between EGFR and either IGF-1R or MET can provide adaptive survival for tumor cells when EGFR is targeted individually (39, 40). Preclinical data highlighting reciprocity for these receptor pairs has spurred the evaluation of combinatorial RTK targeting in the clinic for EGFR inhibitors.

There is growing support for IR as a mitogenic driver for tumor cells, and there are several examples in which IGF-1R or IR can compensate for inhibition of the other in non-transformed cells. Indeed the activity of IGF-2 on IR was first discovered in studying mouse development where it was found that IR, activated by IGF-2, can compensate for IGF-1R disruption to rescue embryonic growth (30). Other studies have described enhanced signaling by insulin when IGF-1R is disrupted in tumor cells (43). Elevated phosphorylation of both IGF-1R and IR is observed in many human tumor cell lines, and it was shown herein that IGF-1R/IR crosstalk is another means exploited by tumor cells to maintain activation of cell survival pathways when IGF-1R is specifically targeted (FIG. 6D). Of particular relevance is our observation that treatment of tumor cell lines with a selective anti-IGF-1R MAb, MAB391, promoted a compensatory increase in phospho-IR in select tumor cell lines. In contrast to observations with the IGF-1R MAb, it was demonstrated that co-targeting IGF-1R and IR with OSI-906 resulted in enhanced inhibition of the IRS1-AKT signaling pathway. Finally, while both OSI-906 and MAB391 achieved efficacy in a human tumor xenograft model expressing only detectable phospho-IGF-1R, only OSI-906 was efficacious in a human tumor xenograft model in which both phospho-IR and phospho-IGF-1R were detectable. In such a setting, it is likely that both IGF-1R and IR are required in tumor cells to mediate growth and or survival signals.

Hyperinsulinemia has been implicated as an increased risk and poor prognosis factor for certain cancers, and one hypothesis is that insulin is driving tumor cell survival through IR-AKT signaling. It was determined herein that treatment with either insulin or IGF-2 could maintain activation of the AKT pathway when IGF-1R was selectively targeted. Insulin concentrations corresponding to mild hyperinsulinemia promoted an increase in phosphorylation of IR and AKT, independent of IGF-1R, and insulin treatment promoted resistance toward inhibition of phospho-AKT by MAB391. Under basal conditions MAB391 promoted a compensatory increase in phospho-IR in tumor cells by approximately two-fold, which was increased further to 12-fold by addition of insulin. IGF-1R-selective drug candidates in clinical development can provoke an increase in systemic insulin levels, therefore the compensatory increase in phospho-IR in response to an anti-IGF-1R antibody in tumor cells may be further enhanced by increased supplies of endocrine insulin ligand (55).

IR, in addition to IGF-1R, can also be activated by IGF-2. MAB391 inhibited IGF-1- or IGF-2-stimulated phospho-IGF-1R. However, while MAB391 inhibited IR when activated by IGF-1, presumably mediated by trans-phosphorylation by IGF-1R within the context of IGF-1R/IR heterodimers, MAB391 had little effect on IGF-2-activated IR signaling. Further, for tumor cells pretreated with MAB391, IGF-2 but not IGF-1, could partially rescue AKT signaling. These data indicate that IGF-2-mediated activation of IR homodimers may compensate for activation of the AKT pathway when IGF-1R is individually targeted. Finally, tumor cell lines with IGF-2 autocrine loops appeared to be especially sensitive to OSI-906 compared to MAB391.

Collectively, these data suggest that co-targeting IGF-1R and IR may deliver enhanced and sustained anti-tumor activity for tumors that are dually reliant on signaling through both of these receptors. Moreover, since rapid resistance to IGF-1R specific antibodies can emerge via increased signaling through IR, dual targeting of IGF-1R and IR by TKIs alone may be efficacious following failure of an anti-IGF-1R antibody. Identifying markers that indicate differential use of these receptors will be important to personalize the use of IGF-1R/IR therapeutics.

### REFERENCES

1. Pollak M. Insulin and insulin-like growth factor signalling in neoplasia. Nat Rev Cancer 2008; 8(12):915-28.
2. Knowlden J M, Hutcheson I R, Barrow D, Gee J M, Nicholson R I. Insulin-like growth factor-I receptor signaling in tamoxifen-resistant breast cancer: a supporting role to the epidermal growth factor receptor. Endocrinology 2005; 146(11):4609-18.
3. Laviola L, Natalicchio A, Giorgino F. The IGF-I signaling pathway. Current pharmaceutical design 2007; 13(7):663-9.
4. De Meyts P, Whittaker J. Structural biology of insulin and IGF1 receptors: implications for drug design. Nat Rev Drug Discov 2002; 1(10):769-83.
5. Peruzzi F, Prisco M, Dews M, et al. Multiple signaling pathways of the insulin-like growth factor 1 receptor in protection from apoptosis. Mol Cell Biol 1999; 19(10):7203-15.
6. Sell C, Rubini M, Rubin R, Liu J P, Efstratiadis A, Baserga R. Simian virus 40 large tumor antigen is unable to transform mouse embryonic fibroblasts lacking type 1 insulin-like growth factor receptor. Proc Natl Acad Sci USA 1993; 90(23):11217-21.
7. Chernicky C L, Yi L, Tan H, Gan S U, Ilan J. Treatment of human breast cancer cells with antisense RNA to the type I insulin-like growth factor receptor inhibits cell growth, suppresses tumorigenesis, alters the metastatic potential, and prolongs survival in vivo. Cancer Gene Ther 2000; 7(3):384-95.
8. Resnicoff M, Coppola D, Sell C, Rubin R, Ferrone S, Baserga R. Growth inhibition of human melanoma cells in nude mice by antisense strategies to the type 1 insulin-like growth factor receptor. Cancer Res 1994; 54(18):4848-50.
9. Cohen B D, Baker D A, Soderstrom C, et al. Combination therapy enhances the inhibition of tumor growth with the fully human anti-type 1 insulin-like growth factor receptor monoclonal antibody CP-751,871. Clin Cancer Res 2005; 11(5):2063-73.
10. Garcia-Echeverria C. Medicinal chemistry approaches to target the kinase activity of IGF-1R. IDrugs 2006; 9(6):415-9.
11. Garcia-Echeverria C, Pearson M A, Marti A, et al. In vivo antitumor activity of NVP-AEW541-A novel, potent, and selective inhibitor of the IGF-IR kinase. Cancer Cell 2004; 5(3):231-9.
12. Haluska P, Carboni J M, Loegering D A, et al. In vitro and in vivo antitumor effects of the dual insulin-like growth factor-I/insulin receptor inhibitor, BMS-554417. Cancer Res 2006; 66(1):362-71.
13. Ji Q S, Mulvihill M J, Rosenfeld-Franklin M, et al. A novel, potent, and selective insulin-like growth factor-I receptor kinase inhibitor blocks insulin-like growth factor-I receptor signaling in vitro and inhibits insulin-like growth factor-I receptor dependent tumor growth in vivo. Mol Cancer Ther 2007; 6(8):2158-67.
14. Maloney E K, McLaughlin J L, Dagdigian N E, et al. An anti-insulin-like growth factor I receptor antibody that is a potent inhibitor of cancer cell proliferation. Cancer Res 2003; 63(16):5073-83.
15. Wang Y, Hailey J, Williams D, et al. Inhibition of insulin-like growth factor-I receptor (IGF-IR) signaling and tumor cell growth by a fully human neutralizing anti-IGF-IR antibody. Mol Cancer Ther 2005; 4(8):1214-21.
16. LeRoith D, Roberts C T, Jr. The insulin-like growth factor system and cancer. Cancer Lett 2003; 195(2):127-37.
17. Rubin R, Baserga R. Insulin-like growth factor-I receptor. Its role in cell proliferation, apoptosis, and tumorigenicity. Lab Invest 1995; 73(3):311-31.
18. Buck E, Eyzaguirre A, Rosenfeld-Franklin M, et al. Feedback mechanisms promote cooperativity for small molecule inhibitors of epidermal and insulin-like growth factor receptors. Cancer Research 2008; in press.
19. Chakravarti A, Loeffler J S, Dyson N J. Insulin-like growth factor receptor I mediates resistance to anti-epidermal growth factor receptor therapy in primary human glioblastoma cells through continued activation of phosphoinositide 3-kinase signaling. Cancer research 2002; 62(1):200-7.
20. Guix M, Faber A C, Wang S E, et al. Acquired resistance to EGFR tyrosine kinase inhibitors in cancer cells is mediated by loss of IGF-binding proteins. The Journal of clinical investigation 2008.
21. Nahta R, Yuan L X, Zhang B, Kobayashi R, Esteva F J. Insulin-like growth factor-I receptor/human epidermal growth factor receptor 2 heterodimerization contributes to trastuzumab resistance of breast cancer cells. Cancer research 2005; 65(23):11118-28.
22. O'Reilly K E, Rojo F, She Q B, et al. mTOR inhibition induces upstream receptor tyrosine kinase signaling and activates Akt. Cancer Res 2006; 66(3):1500-8.
23. Baserga R. Customizing the targeting of IGF-1 receptor. Future oncology (London, England) 2009; 5(1):43-50.
24. Yuen J S, Macaulay V M. Targeting the type 1 insulin-like growth factor receptor as a treatment for cancer. Expert opinion on therapeutic targets 2008; 12(5):589-603.
25. Gualberto A, Pollak M. Emerging role of insulin-like growth factor receptor inhibitors in oncology: early clinical trial results and future directions. Oncogene 2009.
26. Rodon J, DeSantos V, Ferry R J, Jr., Kurzrock R. Early drug development of inhibitors of the insulin-like growth factor-I receptor pathway: lessons from the first clinical trials. Mol Cancer Ther 2008; 7(9):2575-88.
27. Lawrence M C, McKern N M, Ward C W. Insulin receptor structure and its implications for the IGF-1 receptor. Current opinion in structural biology 2007; 17(6):699-705.
28. Ward C W, Garrett T P, McKern N M, et al. The three dimensional structure of the type I insulin-like growth factor receptor. Mol Pathol 2001; 54(3):125-32.
29. Baker J, Liu J P, Robertson E J, Efstratiadis A. Role of insulin-like growth factors in embryonic and postnatal growth. Cell 1993; 75(1):73-82.
30. Louvi A, Accili D, Efstratiadis A. Growth-promoting interaction of IGF-II with the insulin receptor during mouse embryonic development. Developmental biology 1997; 189(1):33-48.
31. Morrione A, Valentinis B, Xu S Q, et al. Insulin-like growth factor II stimulates cell proliferation through the insulin receptor. Proc Natl Acad Sci USA 1997; 94(8):3777-82.
32. Vella V, Sciacca L, Pandini G, et al. The IGF system in thyroid cancer: new concepts. Mol Pathol 2001; 54(3):121-4.
33. Kalli K R, Falowo O I, Bale L K, Zschunke M A, Roche P C, Conover CA. Functional insulin receptors on human epithelial ovarian carcinoma cells: implications for IGF-II mitogenic signaling. Endocrinology 2002; 143(9):3259-67.
34. Giorgino F, Belfiore A, Milazzo G, et al. Overexpression of insulin receptors in fibroblast and ovary cells induces a ligand-mediated transformed phenotype. Mol Endocrinol 1991;5(3):452-9.
35. Frittitta L, Vigneri R, Stampfer M R, Goldline I D. Insulin receptor overexpression in 184B5 human mammary epithelial cells induces a ligand-dependent transformed phenotype. J Cell Biochem 1995; 57(4):666-9.
36. Ma J, Giovannucci E, Pollak M, et al. A prospective study of plasma C-peptide and colorectal cancer risk in men. J Natl Cancer Inst 2004; 96(7):546-53.
37. Jenab M, Riboli E, Cleveland R J, et al. Serum C-peptide, IGFBP-1 and IGFBP-2 and risk of colon and rectal cancers in the European Prospective Investigation into Cancer and Nutrition. Int J Cancer 2007; 121(2):368-76.
38. Kling J. Inhaled insulin's last gasp? Nature biotechnology 2008; 26(5):479-80.
39. Bean J, Brennan C, Shih J Y, et al. MET amplification occurs with or without T790M mutations in EGFR mutant lung tumors with acquired resistance to gefitinib or erlotinib. Proc Natl Acad Sci USA 2007; 104(52):20932-7.
40. Engelman J A, Zejnullahu K, Mitsudomi T, et al. MET amplification leads to gefitinib resistance in lung cancer by activating ERBB3 signaling. Science 2007; 316(5827):1039-43.
41. Fulzele K, DiGirolamo D J, Liu Z, Xu J, Messina J L, Clemens T L. Disruption of the insulin-like growth factor type 1 receptor in osteoblasts enhances insulin signaling and action. J Biol Chem 2007; 282(35):25649-58.
42. Wertheimer E, Spravchikov N, Trebicz M, et al. The regulation of skin proliferation and differentiation in the IR null mouse: implications for skin complications of diabetes. Endocrinology 2001; 142(3):1234-41.
43. Zhang H, Pelzer A M, Kiang D T, Yee D. Down-regulation of type I insulin-like growth factor receptor increases sensitivity of breast cancer cells to insulin. Cancer Res 2007; 67(1):391-7.
44. Buck E, Eyzaguirre A, Haley J D, Gibson N W, Cagnoni P, Iwata K K. Inactivation of Akt by the epidermal growth factor receptor inhibitor erlotinib is mediated by HER-3 in pancreatic and colorectal tumor cell lines and contributes to erlotinib sensitivity. Mol Cancer Ther 2006; 5(8):2051-9.
45. Mulvihill M J, Cooke A, Rosenfeld-Franklin M, et al. Discovery of OSI-906: a selective and orally efficacious dual inhibitor of the IGF-1 receptor and insulin receptor. Future Medicinal Chemistry 2009; 1(6):1153-71.
46. Watters J W, Cheng C, Majumder P K, et al. De novo discovery of a gamma-secretase inhibitor response signature using a novel in vivo breast tumor model. Cancer Res 2009; 69(23):8949-57.
47. Buck E, Eyzaguirre A, Rosenfeld-Franklin M, et al. Feedback mechanisms promote cooperativity for small molecule inhibitors of epidermal and insulin-like growth factor receptors. Cancer Res 2008; 68(20):8322-32.
48. Chen C L, Ip S M, Cheng D, Wong L C, Ngan H Y. Loss of imprinting of the IGF-II and H19 genes in epithelial ovarian cancer. Clin Cancer Res 2000; 6(2):474-9.
49. Cui H, Cruz-Correa M, Giardiello F M, et al. Loss of IGF2 imprinting: a potential marker of colorectal cancer risk. Science 2003; 299(5613):1753-5.
50. Fottner C, Hoeflich A, Wolf E, Weber M M. Role of the insulin-like growth factor system in adrenocortical growth control and carcinogenesis. Hormone and metabolic research Hormon- and Stoffwechselforschung 2004; 36(6):397-405.
51. Giordano T J, Kuick R, Else T, et al. Molecular classification and prognostication of adrenocortical tumors by transcriptome profiling. Clin Cancer Res 2009; 15(2):668-76.
52. Nonomura N, Nishimura K, Miki T, et al. Loss of imprinting of the insulin-like growth factor II gene in renal cell carcinoma. Cancer Res 1997; 57(13):2575-7.
53. Wang Z, Ruan Y B, Guan Y, Liu S H. Expression of IGF-II in early experimental hepatocellular carcinomas and its significance in early diagnosis. World J Gastroenterol 2003; 9(2):267-70.
54. Barlaskar F M, Hammer G D. The molecular genetics of adrenocortical carcinoma. Reviews in endocrine & metabolic disorders 2007; 8(4):343-8.
55. Haluska P, Shaw H M, Batzel G N, et al. Phase I dose escalation study of the anti insulin-like growth factor-I receptor monoclonal antibody CP-751,871 in patients with refractory solid tumors. Clin Cancer Res 2007; 13(19):5834-40.

### ABBREVIATIONS

EGF, epidermal growth factor; EMT, epithelial to mesenchymal transition; NSCLC, non-small cell lung carcinoma; SCC, squamous cell carcinoma; HNSCC, head and neck squamous cell carcinoma; CRC, colorectal cancer; MBC, metastatic breast cancer; INSR or IR, insulin receptor; EGFR, epidermal growth factor receptor; ErbB3, “v-erb-b2 erythroblastic leukemia viral oncogene homolog 3”, also known as HER-3; pHER3, phosphorylated HER3; Erk kinase, Extracellular signal-regulated protein kinase, also known as mitogen-activated protein kinase; pErk, phosphorylated Erk; Brk, Breast tumor kinase (also known as protein tyrosine kinase 6 (PTK6)); LC, liquid chromatography; MS, mass spectrometry; IGF-1, insulin-like growth factor-1; IGF-2, insulin-like growth factor-2; IGF-1R or IGFR, insulin-like growth factor-1 receptor; RTK, receptor-tyrosine kinase; TGFα, transforming growth factor alpha; HB-EGF, heparin-binding epidermal growth factor; LPA, lysophosphatidic acid; TGFα, transforming growth factor alpha; IC₅₀, half maximal inhibitory concentration; RT, room temperature; pY, phosphotyrosine; pPROTEIN, phospho-PROTEIN, “PROTEIN” can be any protein that can be phosphorylated, e.g. EGFR, ERK, HER3, S6 etc; wt, wild-type; PI3K, phosphatidyl inositol-3 kinase; GAPDH, Glyceraldehyde 3-phosphate dehydrogenase, PMID, PubMed Unique Identifier; NCBI, National Center for Biotechnology Information; NCI, National Cancer Institute; MSKCC, Memorial Sloan Kettering Cancer Center; ECACC, European Collection of Cell Cultures; ATCC, American Type Culture Collection.

### INCORPORATION BY REFERENCE

All patents, published patent applications and other references disclosed herein are hereby expressly incorporated herein by reference.

### EQUIVALENTS

Those skilled in the art will recognize, or be able to ascertain, using no more than routine experimentation, many equivalents to specific embodiments of the invention described specifically herein. Such equivalents are intended to be encompassed in the scope of the following claims.

1-38. (canceled)

39. A method of identifying patients with cancer who are likely to benefit from treatment with an IGF-1R kinase inhibitor that inhibits both IGF-1R and IR kinases, comprising:
assessing the expression level of the five gene transcripts IGF-1R, IR, IR-A, IGF-1 and IGF-2 in tumor cells from a sample of a patient's tumor;
determining an expression level index for the five gene transcripts by adding the expression level values for each of the five transcripts;
determining that if the value of the expression level index for the tumor cells of the sample is equal to or greater than the value of the expression level index for RDES tumor cells or SK-N-AS tumor cells determined by identical methods, the patient is likely to benefit from treatment with an IGF-1R kinase inhibitor that inhibits both IGF-1R and IR kinases.

40. The method of claim 39, wherein the tumor cells are from a cancer selected from myeloma, NSCLC, ACC, ovarian cancer, HNSCC, colon cancer, Ewing's sarcoma, rhabdomyosarcoma, neuroblastoma, pancreatic cancer, or breast cancer.

41. The method of claim 39, wherein the IGF-1R kinase inhibitor that inhibits both IGF-1R and IR kinases is OSI-906.

42. A method for treating a patient with cancer, comprising administering to said patient a therapeutically effective amount of an IGF-1R kinase inhibitor that inhibits both IGF-1R and IR kinases, if the patient has been diagnosed to be potentially responsive to such an IGF-1R kinase inhibitor, by assessing the expression level of the five gene transcripts IGF-1R, IR, IR-A, IGF-1 and IGF-2 in the tumor cells of the cancer; determining an expression level index for the five gene transcripts by adding the expression level values for each of the five transcripts; and determining that the value of the expression level index for the tumor cells is equal to or greater than the value of the expression level index for RDES tumor cells or SK-N-AS tumor cells determined by identical methods.

43. The method of claim 42, wherein the tumor cells are from a cancer selected from myeloma, NSCLC, ACC, ovarian cancer, HNSCC, colon cancer, Ewing's sarcoma, rhabdomyosarcoma, neuroblastoma, pancreatic cancer, or breast cancer.

44. The method of claim 42, wherein the IGF-1R kinase inhibitor that inhibits both IGF-1R and IR kinases is OSI-906.

45. The method of claim 42, wherein one or more additional anti-cancer agents are co-administered simultaneously or sequentially with the IGF-1R kinase inhibitor.

46. A method of identifying patients with cancer who are likely to benefit from treatment with an IGF-1R kinase inhibitor that inhibits both IGF-1R and IR kinases, but would likely not respond to therapy with an anti-IGF-1R antibody, comprising:
assessing the expression level of the five gene transcripts IGF-1R, IR, IR-A, IGF-1 and IGF-2 in tumor cells from a sample of a patient's tumor;
determining an expression level index for the five gene transcripts by adding the expression level values for each of the five transcripts;
determining that if the value of the expression level index for the tumor cells of the sample is equal to or greater than the value of the expression level index for RDES or SK-N-AS tumor cells as determined by identical methods, the patient is likely to benefit from treatment with an IGF-1R kinase inhibitor that inhibits both IGF-1R and IR kinases;
determining that if the value of the sum of expression levels for IR and IR-A for the tumor cells of the sample is equal to or greater than the sum of expression levels for IR and IR-A for GEO or A673 tumor cells as determined by identical methods, the patient is not likely to benefit from treatment with an anti-IGF-1R antibody,
thus identifying patients with cancer who are likely to benefit from treatment with an IGF-1R kinase inhibitor that inhibits both IGF-1R and IR kinases, but would likely not respond to therapy with an anti-IGF-1R antibody.

47. The method of claim 46, wherein the tumor cells are from a cancer selected from myeloma, NSCLC, ACC, ovarian cancer, HNSCC, colon cancer, Ewing's sarcoma, rhabdomyosarcoma, neuroblastoma, pancreatic cancer, or breast cancer.

48. The method of claim 46, wherein the IGF-1R kinase inhibitor that inhibits both IGF-1R and IR kinases is OSI-906.

49. A method for treating a patient with cancer, comprising administering to said patient a therapeutically effective amount of an IGF-1R kinase inhibitor that inhibits both IGF-1R and IR kinases, if the patient has been diagnosed to be potentially responsive to such an IGF-1R kinase inhibitor, by assessing the expression level of the five gene transcripts IGF-1R, IR, IR-A, IGF-1 and IGF-2 in the tumor cells of the cancer; determining an expression level index for the five gene transcripts by adding the expression level values for each of the five transcripts; and determining that the value of the expression level index for the tumor cells is equal to or greater than the value of the expression level index for RDES tumor cells or SK-N-AS tumor cells determined by identical methods, and if the patient has beens diagnosed to be potentially unresponsive to treatment an anti-IGF-1R antibody by determining that the value of the sum of expression levels for IR and IR-A for the tumor cells of the cancer is equal to or greater than the sum of expression levels for IR and IR-A for GEO or A673 tumor cells as determined by identical methods.

50. The method of claim 49, wherein the tumor cells are from a cancer selected from myeloma, NSCLC, ACC, ovarian cancer, HNSCC, colon cancer, Ewing's sarcoma, rhabdomyosarcoma, neuroblastoma, pancreatic cancer, or breast cancer.

51. The method of claim 49, wherein the IGF-1R kinase inhibitor that inhibits both IGF-1R and IR kinases is OSI-906.

52. The method of claim 49, wherein one or more additional anti-cancer agents are co-administered simultaneously or sequentially with the IGF-1R kinase inhibitor.

53. A method of identifying patients with cancer who are likely to benefit from treatment with an IGF-1R kinase inhibitor that inhibits both IGF-1R and IR kinases, comprising:
assessing the expression level of the gene transcripts IGF-1R, IR, IR-A, IGF-1 and IGF-2 in tumor cells from a sample of a patient's tumor;
determining that if the tumor cells of the sample express IGF-1R, express IR and/or IR-A, and if the value of the sum of expression levels for IGF-1 and IGF-2 for the tumor cells of the sample greater than the sum of expression levels for IGF-1 and IGF-2 for RD tumor cells as determined by identical methods, the patient is likely to benefit from treatment with an IGF-1R kinase inhibitor that inhibits both IGF-1R and IR kinases.

54. The method of claim 53, wherein the tumor cells are from a cancer selected from myeloma, NSCLC, ACC, ovarian cancer, HNSCC, colon cancer, Ewing's sarcoma, rhabdomyosarcoma, neuroblastoma, pancreatic cancer, or breast cancer.

55. The method of claim 53, wherein the IGF-1R kinase inhibitor that inhibits both IGF-1R and IR kinases is OSI-906.

56. A method for treating a patient with cancer, comprising administering to said patient a therapeutically effective amount of an IGF-1R kinase inhibitor that inhibits both IGF-1R and IR kinases if the patient has been determined to be likely to benefit from treatment with such an inhibitor by assessing the expression level of the gene transcripts IGF-1R, IR, IR-A, IGF-1 and IGF-2 in the tumor cells of the patient's tumor; and determining that the tumor cells of the patient's tumor express IGF-1R, express IR and/or IR-A, and the value of the sum of expression levels for IGF-1 and IGF-2 for the tumor cells of the patient's tumor is greater than the sum of expression levels for IGF-1 and IGF-2 for RD tumor cells as determined by identical methods.

57. The method of claim 56, wherein the tumor cells are from a cancer selected from myeloma, NSCLC, ACC, ovarian cancer, HNSCC, colon cancer, Ewing's sarcoma, rhabdomyosarcoma, neuroblastoma, pancreatic cancer, or breast cancer.

58. The method of claim 56, wherein the IGF-1R kinase inhibitor that inhibits both IGF-1R and IR kinases is OSI-906.

59. The method of claim 56, wherein one or more additional anti-cancer agents are co-administered simultaneously or sequentially with the IGF-1R kinase inhibitor.
